SEQUENCE LISTING (115 sequences)

SEQ ID NO: 1 - protein, 5 aa, Mus sp.
DYSMH

SEQ ID NO: 2 - protein, 17 aa, Mus sp.
WINTETGEPTYADDFKG

SEQ ID NO: 3 - protein, 13 aa, Mus sp.
PYYDYVSYYAMDY

SEQ ID NO: 4 - protein, 122 aa, Mus sp.
QIQLVQSGPELKKPGETVKISCKASGYTFTDYSMHWVKQAPGKGLKWMGWINTETGEPTYADDFKGRFAFSLETSASTAYLQINNLKNEDTATYFCANPYYDYVSYYAMDYWGHGTSVTVSS

SEQ ID NO: 5 - protein, 122 aa, Artificial Sequence
  source: /note=“Description of Artificial Sequence Synthetic polypeptide”
QVQLVQSGSELKKPGASVKVSCKASGYTFTDYSMHWVRQAPGQGLKWMGWINTETGEPTYADDFKGRFVFSLDTSVSTAYLQISSLKAEDTAVYYCANPYYDYVSYYAMDYWGQGTTVTVSS

SEQ ID NO: 6 - DNA, 458 bp, Artificial Sequence
  source: /note=“Description of Artificial Sequence Synthetic polynucleotide”
actagtaccaccatggcttgggtgtggaccttgctattcctgatggcagctgcccaaagtatccaagcacaggttcagttggtgcagtctggatctgagctgaagaagcctggagcctcagtcaaggtttcctgcaaggcttctggttataccttcacagactattcaatgcactgggtgcgacaggctccaggacaaggtttaaagtggatgggctggataaacactgagactggtgagccaacatatgcagatgacttcaagggacggtttgtcttctctttggacacctctgtcagcactgcctatttgcagatcagcagcctcaaagctgaggacacggctgtgtattactgtgctaatccctactatgattacgtctcttactatgctatggactactggggtcagggaaccacggtcaccgtctcctcaggtaagaatggcctctcaagctt

SEQ ID NO: 7 - protein, 11 aa, Mus sp.
KASQDVSTAVA

SEQ ID NO: 8 - protein, 7 aa, Mus sp.
SASYLYT

SEQ ID NO: 9 - protein, 9 aa, Mus sp.
QQHYSTPRT

SEQ ID NO: 10 - protein, 107 aa, Mus sp.
DIVMTQSHKFMSTSVRDRVSITCKASQDVSTAVAWYQQKPGQSPKLLIYSASYLYTGVPDRFTGSGSGTDFTFTISSVQAEDLAVYYCQQHYSTPRTFGGGTKLEIK

SEQ ID NO: 11 - protein, 107 aa, Artificial Sequence
  source: /note=“Description of Artificial Sequence Synthetic polypeptide”
DIQMTQSPSSLSASVGDRVTITCKASQDVSTAVAWYQQKPGKAPKLLIYSASYLYTGVPSRFSGSGSGTDFTFTISSLQPEDIATYYCQQHYSTPRTFGQGTKLEIK

SEQ ID NO: 12 - DNA, 416 bp, Artificial Sequence
  source: /note=“Description of Artificial Sequence Synthetic polynucleotide”
gctagcaccaccatggagtcacagattcaggtctttgtattcgtgtttctctggttgtctggtgttgacggagacattcagatgacccagtctccatcctccctgtccgcatcagtgggagacagggtcaccatcacctgcaaggccagtcaggatgtgagtactgctgtagcctggtatcaacagaaaccaggaaaagcccctaaactactgatttactcggcatcctacctctacactggagtcccttcacgcttcagtggcagtggatctgggacggatttcactttcaccatcagcagtctgcagcctgaagacattgcaacatattactgtcagcaacattatagtactcctcggacgttcggtcagggcaccaagctggaaatcaaacgtaagtagaatccaaagaattc

SEQ ID NO: 13 - protein, 5 aa, Mus sp.
SHDMS

SEQ ID NO: 14 - protein, 17 aa, Mus sp.
AINSDGGSTYYPDTMER

SEQ ID NO: 15 - protein, 11 aa, Mus sp.
HYDDYYAWFAY

SEQ ID NO: 16 - protein, 120 aa, Mus sp.
EVQLVESGGGLVQPGESLKLSCESNEYEFPSHDMSWVRKTPEKRLELVAAINSDGGSTYYPDTMERRFIISRDNTKKTLYLQMSSLRSEDTALYYCARHYDDYYAWFAYWGQGTLVTVSA

SEQ ID NO: 17 - protein, 120 aa, Artificial Sequence
  source: /note=“Description of Artificial Sequence Synthetic polypeptide”
EVQLVESGGGLVQPGGSLRLSCAASEYEFPSHDMSWVRQAPGKGLELVAAINSDGGSTYYPDTMERRFTISRDNAKNSLYLQMNSLRAEDTAVYYCARHYDDYYAWFAYWGQGTMVTVSS

SEQ ID NO: 18 - DNA, 451 bp, Artificial Sequence
  source: /note=“Description of Artificial Sequence Synthetic polynucleotide”
actagtaccaccatggacttcgggctcagcttggttttccttgtccttattttaaaaagtgtacagtgtgaggtgcagctggtggagtctgggggaggcttagtgcagcctggagggtccctgagactctcctgtgcagcctctgaatacgagttcccttcccatgacatgtcttgggtccgccaggctccggggaaggggctggagttggtcgcagccattaatagtgatggtggtagcacctactatccagacaccatggagagacgattcaccatctccagagacaatgccaagaactcactgtacctgcaaatgaacagtctgagggccgaggacacagccgtgtattactgtgcaagacactatgatgattactacgcctggtttgcttactggggccaagggactatggtcactgtctcttcaggtgagtcctaacttcaagctt

SEQ ID NO: 19 - protein, 15 aa, Mus sp.
RASKSVSTSGYSYMH

SEQ ID NO: 20 - protein, 7 aa, Mus sp.
LASNLES

SEQ ID NO: 21 - protein, 9 aa, Mus sp.
QHSRELPLT

SEQ ID NO: 22 - protein, 111 aa, Mus sp.
DIVLTQSPASLAVSLGQRATISCRASKSVSTSGYSYMHWYQQKPGQPPKLLIYLASNLESGVPARFSGSGSGTDFTLNIHPVEEEDAATYYCQHSRELPLTFGAGTKLELK

SEQ ID NO: 23 - protein, 111 aa, Artificial Sequence
  source: /note=“Description of Artificial Sequence Synthetic polypeptide”
EIVLTQSPATLSLSPGERATLSCRASKSVSTSGYSYMHWYQQKPGQAPRLLIYLASNLESGVPARFSGSGSGTDFTLTISSLEPEDFAVYYCQHSRELPLTFGGGTKVEIK

SEQ ID NO: 24 - DNA, 428 bp, Artificial Sequence
  source: /note=“Description of Artificial Sequence Synthetic polynucleotide”
gctagcaccaccatggagacagacacactcctgttatgggtactgctgctctgggttccaggttccactggtgaaattgtgctgacacagtctcctgctaccttatctttgtctccaggggaaagggccaccctctcatgcagggccagcaaaagtgtcagtacatctggctatagttatatgcactggtaccaacagaaaccaggacaggctcccagactcctcatctatcttgcatccaacctagaatctggggtccctgccaggttcagtggcagtgggtctgggacagacttcaccctcaccatcagcagcctagagcctgaggattttgcagtttattactgtcagcacagtagggagcttccgctcacgttcggcggagggaccaaggtcgagatcaaacgtaagtacacttttctgaattc

SEQ ID NO: 25 - protein, 5 aa, Mus sp.
DAWMD

SEQ ID NO: 26 - protein, 19 aa, Mus sp.
EIRSKANNHATYYAESVNG

SEQ ID NO: 27 - protein, 8 aa, Mus sp.
GEVFYFDY

SEQ ID NO: 28 - DNA, 414 bp, Mus sp.
atgtacttgggactgaactatgtattcatagtttttctcttaaatggtgtccagagtgaagtgaagcttgaggagtctggaggaggcttggtgcaacctggaggatccatgaaactctcttgtgctgcctctggattcacttttagtgacgcctggatggactgggtccgccagtctccagagaaggggcttgagtgggttgctgaaattagaagcaaagctaataatcatgcaacatactatgctgagtctgtgaatgggaggttcaccatctcaagagatgattccaaaagtagtgtctacctgcaaatgaacagcttaagagctgaagacactggcatttattactgtacgtggggggaagtgttctactttgactactggggccaaggcaccactctcacagtctcctca

SEQ ID NO: 29 - protein, 138 aa, Mus sp.
MYLGLNYVFIVFLLNGVQSEVKLEESGGGLVQPGGSMKLSCAASGFTFSDAWMDWVRQSPEKGLEWVAEIRSKANNHATYYAESVNGRFTISRDDSKSSVYLQMNSLRAEDTGIYYCTWGEVFYFDYWGQGTTLTVSS

SEQ ID NO: 30 - DNA, 448 bp, Artificial Sequence
  source: /note=“Description of Artificial Sequence Synthetic polynucleotide”
actagtaccaccatgtacttgggactgaactatgtattcatagtttttctcttaaatggtgtccagagtgaagtgaagctggaggagtctggaggaggcttggtgcaacctggaggatccatgaaactctcttgtgctgcctctggattcacttttagtgacgcctggatggactgggtccgccagtctccagagaaggggcttgagtgggttgctgaaattagaagcaaagctaataatcatgcaacatactatgctgagtctgtgaatgggaggttcaccatctcaagagatgattccaaaagtagtgtctacctgcaaatgaacagcttaagagctgaagacactggcatttattactgtacgtggggggaagtgttctactttgactactggggccaaggcaccactctcacagtctcctcaggtgagtccttaaaacaagctt

SEQ ID NO: 31 - protein, 138 aa, Artificial Sequence; identical to SEQ ID NO: 29
  source: /note=“Description of Artificial Sequence Synthetic polypeptide”

SEQ ID NO: 32 - protein, 11 aa, Mus sp.
KSSQDINKYIA

SEQ ID NO: 33 - protein, 7 aa, Mus sp.
YTSTLQP

SEQ ID NO: 34 - protein, 8 aa, Mus sp.
LQYDNLLT

SEQ ID NO: 35 - DNA, 378 bp, Mus sp.
atgagaccgtctattcagttcctggggctcttgttgttctggcttcatggtgctcagtgtgacatccagatgacacagtctccatcctcactgtctgcatctctgggaggcaaagtcaccatcacttgcaagtcaagccaagacattaacaagtatatagcttggtaccaacacaagcctggaaaaggtcctaggctgctcatacattacacatctacattacagccaggcatcccatcaaggttcagtggaagtgggtctgggagagattattccttcagcatcagcaacctggagcctgaagatattgcaacttattattgtctacagtatgataatcttctcacgttcggtgctgggaccaagctggagctgaaa

SEQ ID NO: 36 - protein, 126 aa, Mus sp.
MRPSIQFLGLLLFWLHGAQCDIQMTQSPSSLSASLGGKVTITCKSSQDINKYIAWYQHKPGKGPRLLIHYTSTLQPGIPSRFSGSGSGRDYSFSISNLEPEDIATYYCLQYDNLLTFGAGTKLELK

SEQ ID NO: 37 - DNA, 413 bp, Artificial Sequence
  source: /note=“Description of Artificial Sequence Synthetic polynucleotide”
gctagcaccaccatgagaccgtctattcagttcctggggctcttgttgttctggcttcatggtgctcagtgtgacatccagatgacacagtctccatcctcactgtctgcatctctgggaggcaaagtcaccatcacttgcaagtcaagccaagacattaacaagtatatagcttggtaccaacacaagcctggaaaaggtcctaggctgctcatacattacacatctacattacagccaggcatcccatcaaggttcagtggaagtgggtctgggagagattattccttcagcatcagcaacctggagcctgaagatattgcaacttattattgtctacagtatgataatcttctcacgttcggtgctgggaccaagctggagctgaaacgtaagtacacttttctgaattc

SEQ ID NO: 38 - protein, 126 aa, Artificial Sequence; identical to SEQ ID NO: 36
  source: /note=“Description of Artificial Sequence Synthetic polypeptide”

SEQ ID NO: 39 - DNA, 20 bp, Artificial Sequence
  source: /note=“Description of Artificial Sequence Synthetic primer”
cgctgttttgacctccatag

SEQ ID NO: 40 - DNA, 20 bp, Artificial Sequence
  source: /note=“Description of Artificial Sequence Synthetic primer”
tgaaagatgagctggaggac

SEQ ID NO: 41 - DNA, 20 bp, Artificial Sequence
  source: /note=“Description of Artificial Sequence Synthetic primer”
ctttcttgtccaccttggtg

SEQ ID NO: 42 - DNA, 19 bp, Artificial Sequence
  source: /note=“Description of Artificial Sequence Synthetic primer”
gctgtcctacagtcctcag

SEQ ID NO: 43 - DNA, 18 bp, Artificial Sequence
  source: /note=“Description of Artificial Sequence Synthetic primer”
acgtgccaagcatcctcg

SEQ ID NO: 44 - DNA, 1407 bp, Artificial Sequence
  source: /note=“Description of Artificial Sequence Synthetic polynucleotide”
atgtacttgggactgaactatgtattcatagtttttctcttaaatggtgtccagagtgaagtgaagctggaggagtctggaggaggcttggtgcaacctggaggatccatgaaactctcttgtgctgcctctggattcacttttagtgacgcctggatggactgggtccgccagtctccagagaaggggcttgagtgggttgctgaaattagaagcaaagctaataatcatgcaacatactatgctgagtctgtgaatgggaggttcaccatctcaagagatgattccaaaagtagtgtctacctgcaaatgaacagcttaagagctgaagacactggcatttattactgtacgtggggggaagtgttctactttgactactggggccaaggcaccactctcacagtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 45 - protein, 469 aa, Artificial Sequence [sequence omitted]

SEQ ID NO: 46 - DNA, 702 bp, Artificial Sequence
  source: /note=“Description of Artificial Sequence Synthetic polynucleotide”
atgagaccgtctattcagttcctggggctcttgttgttctggcttcatggtgctcagtgtgacatccagatgacacagtctccatcctcactgtctgcatctctgggaggcaaagtcaccatcacttgcaagtcaagccaagacattaacaagtatatagcttggtaccaacacaagcctggaaaaggtcctaggctgctcatacattacacatctacattacagccaggcatcccatcaaggttcagtggaagtgggtctgggagagattattccttcagcatcagcaacctggagcctgaagatattgcaacttattattgtctacagtatgataatcttctcacgttcggtgctgggaccaagctggagctgaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 47 - protein, 233 aa, Artificial Sequence
  source: /note=“Description of Artificial Sequence Synthetic polypeptide”
MRPSIQFLGLLLFWLHGAQCDIQMTQSPSSLSASLGGKVTITCKSSQDINKYIAWYQHKPGKGPRLLIHYTSTLQPGIPSRFSGSGSGRDYSFSISNLEPEDIATYYCLQYDNLLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 48 - protein, 452 aa, Artificial Sequence
  source: /note=“Description of Artificial Sequence Synthetic polypeptide”
QVQLVQSGSELKKPGASVKVSCKASGYTFTDYSMHWVRQAPGQGLKWMGWINTETGEPTYADDFKGRFVFSLDTSVSTAYLQISSLKAEDTAVYYCANPYYDYVSYYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 49 - protein, 214 aa, Artificial Sequence
  source: /note=“Description of Artificial Sequence Synthetic polypeptide”
DIQMTQSPSSLSASVGDRVTITCKASQDVSTAVAWYQQKPGKAPKLLIYSASYLYTGVPSRFSGSGSGTDFTFTISSLQPEDIATYYCQQHYSTPRTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 50 - protein, 446 aa, Artificial Sequence
  source: /note=“Description of Artificial Sequence Synthetic polypeptide”
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 51 - protein, 218 aa, Artificial Sequence
  source: /note=“Description of Artificial Sequence Synthetic polypeptide”
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 52 - protein, 120 aa, Artificial Sequence [sequence omitted]

SEQ ID NO: 53 - protein, 111 aa, Artificial Sequence [sequence omitted]

SEQ ID NO: 54 - protein, 5 aa, Artificial Sequence
  source: /note=“Description of Artificial Sequence Synthetic peptide”
NYYMY

SEQ ID NO: 55 - protein, 17 aa, Artificial Sequence
  source: /note=“Description of Artificial Sequence Synthetic peptide”
GINPSNGGTNFNEKFKN

SEQ ID NO: 56 - protein, 11 aa, Artificial Sequence
  source: /note=“Description of Artificial Sequence Synthetic peptide”
RDYRFDMGFDY

SEQ ID NO: 57 - protein, 15 aa, Artificial Sequence
  source: /note=“Description of Artificial Sequence Synthetic peptide”
RASKGVSTSGYSYLH

SEQ ID NO: 58 - protein, 7 aa, Artificial Sequence
  source: /note=“Description of Artificial Sequence Synthetic peptide”
LASYLES

SEQ ID NO: 59 - protein, 9 aa, Artificial Sequence
  source: /note=“Description of Artificial Sequence Synthetic peptide”
QHSRDLPLT

SEQ ID NO: 60 - DNA, 435 bp, Artificial Sequence
  source: /note=“Description of Artificial Sequence Synthetic hPD-1.08A heavy chain variable region sequence”
atgrgatggagctgtatcatkctctttttggtagcaacagctacaggtgtccactcccaggtccaactgcagcagcctggggctgaactggtgaagcctggggcttcagtgaagttgtcctgcaaggcctctggctacaccttcaccagttattatctgtactggatgaaacagaggcctggacaaggccttgagtggattgggggggttaatcctagtaatggtggtactaacttcagtgagaagttcaagagcaaggccacactgactgtagacaaatcctccagcacagcctacatgcaactcagcagcctgacatctgaggactctgcggtctattactgtacaagaagggattctaactacgacgggggctttgactactggggccaaggcactactctcacagtctcctcagccaaaacgacaccccca

SEQ ID NO: 61 - DNA, 453 bp, Artificial Sequence
  source: /note=“Description of Artificial Sequence Synthetic hPD-1.08A light chain variable region sequence”
atggagacagacacactcctgctatgggtactgctgctctgggttccaggttccactggtgacattgtgctgacacagtctcctacttccttagctgtatctctggggcagagggccaccatctcatgcagggccagcaaaagtgtcagtacatctggctttagttatttgcactggtaccaacagaaaccaggacagccacccaaactcctcatctttcttgcatccaacctagagtctggggtccctgccaggttcagtggcagtgggtctgggacagacttcaccctcaacatccatcctgtggaggaggaggacgctgcaacctattattgtcagcacagttgggagcttccgctcacgttcggtgctgggaccaagctggagctgaaacgggctgatgctgcaccaactgtatccatcttcccaccatccagtaagcttgggaagggc

SEQ ID NO: 62 - DNA, 464 bp, Artificial Sequence
  source: /note=“Description of Artificial Sequence Synthetic hPD-1.09A heavy chain variable region sequence”
atgraatgcagctgggttatyctctttttggtagcaacagctacaggtgtccactcccaggtccaactgcagcagcctggggctgaactggtgaagcctgggacttcagtgaagttgtcctgcaaggcttctggctacaccttcaccaactactatatgtactgggtgaagcagaggcctggacaaggccttgagtggattggggggattaatcctagcaatggtggtactaacttcaatgagaagttcaagaacaaggccacactgactgtagacagttcctccagcacaacctacatgcaactcagcagcctgacatctgaggactctgcggtctattactgtacaagaagggattataggttcgacatgggctttgactactggggccaaggcaccactctcacagtctcctcagccaaaacgacacccccatccgtytatcccbtggcccctggaagctt

SEQ ID NO: 63 - DNA, 438 bp, Artificial Sequence
  source: /note=“Description of Artificial Sequence Synthetic hPD-1.09A light chain variable region sequence”
atggagwcagacacactsctgytatgggtactgctgctctgggttccaggttccactggcgacattgtgctgacacagtctcctgcttccttagctgtatctctgggacagagggccgccatctcatgcagggccagcaaaggtgtcagtacatctggctatagttatttgcactggtaccaacagaaaccaggacagtcacccaaactcctcatctatcttgcatcctacctagaatctggggtccctgccaggttcagtggcagtgggtctgggacagacttcaccctcaacatccatcctgtggaggaggaggatgctgcaacctattactgtcagcacagtagggaccttccgctcacgttcggtactgggaccaagctggagctgaaacgggctgatgctgcaccaactgtatccatcttcccaccatccagt

SEQ ID NO: 64 - protein, 122 aa, Artificial Sequence
  source: /note=“Description of Artificial Sequence Synthetic hPD-1.08A heavy chain variable region sequence”
QVQLQQPGAELVKPGASVKLSCKASGYTFTSYYLYWMKQRPGQGLEWIGGVNPSNGGTNFSEKFKSKATLTVDKSSSTAYMQLSSLTSEDSAVYYCTRRDSNYDGGFDYWGQGTTLTVSSAK

SEQ ID NO: 65 - protein, 111 aa, Artificial Sequence
  source: /note=“Description of Artificial Sequence Synthetic hPD-1.08A light chain variable region sequence”
DIVLTQSPTSLAVSLGQRATISCRASKSVSTSGFSYLHWYQQKPGQPPKLLIFLASNLESGVPARFSGSGSGTDFTLNIHPVEEEDAATYYCQHSWELPLTFGAGTKLELK

SEQ ID NO: 66 - protein, 122 aa, Artificial Sequence
  source: /note=“Description of Artificial Sequence Synthetic hPD-1.09A heavy chain variable region sequence”
QVQLQQPGAELVKPGTSVKLSCKASGYTFTNYYMYWVKQRPGQGLEWIGGINPSNGGTNFNEKFKNKATLTVDSSSSTTYMQLSSLTSEDSAVYYCTRRDYRFDMGFDYWGQGTTLTVSSAK

SEQ ID NO: 67 - protein, 111 aa, Artificial Sequence
  source: /note=“Description of Artificial Sequence Synthetic hPD-1.09A light chain variable region sequence”
DIVLTQSPASLAVSLGQRAAISCRASKGVSTSGYSYLHWYQQKPGQSPKLLIYLASYLESGVPARFSGSGSGTDFTLNIHPVEEEDAATYYCQHSRDLPLTFGTGTKLELK

SEQ ID NO: 68 - protein, 15 aa, Artificial Sequence
  source: /note=“Description of Artificial Sequence Synthetic hPD-1.08A light chain CDR1 sequence”
RASKSVSTSGFSYLH

SEQ ID NO: 69 - protein, 7 aa, Artificial Sequence
  source: /note=“Description of Artificial Sequence Synthetic hPD-1.08A light chain CDR2 sequence”
LASNLES

SEQ ID NO: 70 - protein, 9 aa, Artificial Sequence
  source: /note=“Description of Artificial Sequence Synthetic hPD-1-08A light chain CDR3 sequence”
QHSWELPLT

SEQ ID NO: 71 - protein, 5 aa, Artificial Sequence
  source: /note=“Description of Artificial Sequence Synthetic hPD-1.08A heavy chain CDR1 sequence”
SYYLY

SEQ ID NO: 72 - protein, 17 aa, Artificial Sequence
  source: /note=“Description of Artificial Sequence Synthetic hPD-1.08A heavy chain CDR2 sequence”
GVNPSNGGTNFSEKFKS

SEQ ID NO: 73 - protein, 11 aa, Artificial Sequence
  source: /note=“Description of Artificial Sequence Synthetic hPD-1.08A heavy chain CDR3 sequence”
RDSNYDGGFDY

SEQ ID NO: 74 - protein, 15 aa, Artificial Sequence
  source: /note=“Description of Artificial Sequence Synthetic hPD-1.09A light chain CDR1 sequence”
RASKGVSTSGYSYLH

SEQ ID NO: 75 - protein, 7 aa, Artificial Sequence
  source: /note=“Description of Artificial Sequence Synthetic hPD-1.09A light chain CDR2 sequence”
LASYLES

SEQ ID NO: 76 - protein, 9 aa, Artificial Sequence
  source: /note=“Description of Artificial Sequence Synthetic hPD-1.09A light chain CDR3 sequence”
QHSRDLPLT

SEQ ID NO: 77 - protein, 5 aa, Artificial Sequence
  source: /note=“Description of Artificial Sequence Synthetic hPD-1.09A heavy chain CDR1 sequence”
NYYMY

SEQ ID NO: 78 - protein, 17 aa, Artificial Sequence
  source: /note=“Description of Artificial Sequence Synthetic hPD-1.09A heavy chain CDR2 sequence”
GINPSNGGTNFNEKFKN

SEQ ID NO: 79 - protein, 11 aa, Artificial Sequence
  source: /note=“Description of Artificial Sequence Synthetic hPD-1.09A heavy chain CDR3 sequence”
RDYRFDMGFDY

SEQ ID NO: 80 - DNA, 417 bp, Artificial Sequence
  source: /note=“Description of Artificial Sequence Synthetic 109A-H heavy chain variable region sequence”
  sig_peptide (1)..(57)
atggactggacctggagcatccttttcttggtggcagcaccaacaggagcccactcccaagtgcagctggtgcagtctggagttgaagtgaagaagcccggggcctcagtgaaggtctcctgcaaggcttctggctacacctttaccaactactatatgtactgggtgcgacaggcccctggacaagggcttgagtggatgggagggattaatcctagcaatggtggtactaacttcaatgagaagttcaagaacagagtcaccttgaccacagactcatccacgaccacagcctacatggaactgaagagcctgcaatttgacgacacggccgtttattactgtgcgagaagggattataggttcgacatgggctttgactactggggccaagggaccacggtcaccgtctcgagc

SEQ ID NO: 81 - DNA, 417 bp, Artificial Sequence
  source: /note=“Description of Artificial Sequence Synthetic Codon optimized 109A-H heavy chain variable region sequence”
  sig_peptide (1)..(57)
atggactggacctggtctatcctgttcctggtggccgctcctaccggcgctcactcccaggtgcagctggtgcagtccggcgtggaggtgaagaagcctggcgcctccgtcaaggtgtcctgcaaggcctccggctacaccttcaccaactactacatgtactgggtgcggcaggctcccggccagggactggagtggatgggcggcatcaacccttccaacggcggcaccaacttcaacgagaagttcaagaaccgggtgaccctgaccaccgactcctccaccaccaccgcctacatggagctgaagtccctgcagttcgacgacaccgccgtgtactactgcgccaggcgggactaccggttcgacatgggcttcgactactggggccagggcaccaccgtgaccgtgtcctcc

SEQ ID NO: 82 - DNA, 1398 bp, Artificial Sequence
  source: /note=“Description of Artificial Sequence Synthetic Codon optimized 409A-H heavy chain full length sequence”
  sig_peptide (1)..(57)
atggccgtgctgggcctgctgttctgcctggtgaccttcccttcctgcgtgctgtcccaggtgcagctggtgcagtccggcgtggaggtgaagaagcctggcgcctccgtcaaggtgtcctgtaaggcctccggctacaccttcaccaactactacatgtactgggtgcggcaggccccaggccagggactggagtggatgggcggcatcaacccttccaacggcggcaccaacttcaacgagaagttcaagaaccgggtgaccctgaccaccgactcctccaccacaaccgcctacatggaactgaagtccctgcagttcgacgacaccgccgtgtactactgcgccaggcgggactaccggttcgacatgggcttcgactactggggccagggcaccaccgtgaccgtgtcctccgctagcaccaagggcccttccgtgttccctctggccccttgctcccggtccacctccgagtccaccgccgctctgggctgtctggtgaaggactacttccctgagcctgtgaccgtgagctggaactctggcgccctgacctccggcgtgcacaccttccctgccgtgctgcagtcctccggcctgtactccctgtcctccgtggtgaccgtgccttcctcctccctgggcaccaagacctacacctgcaacgtggaccacaagccttccaacaccaaggtggacaagcgggtggagtccaagtacggccctccttgccctccctgccctgcccctgagttcctgggcggaccctccgtgttcctgttccctcctaagcctaaggacaccctgatgatctcccggacccctgaggtgacctgcgtggtggtggacgtgtcccaggaagatcctgaggtccagttcaattggtacgtggatggcgtggaggtgcacaacgccaagaccaagcctcgggaggaacagttcaactccacctaccgggtggtgtctgtgctgaccgtgctgcaccaggactggctgaacggcaaggaatacaagtgcaaggtcagcaacaagggcctgccctcctccatcgagaaaaccatctccaaggccaagggccagcctcgcgagcctcaggtgtacaccctgcctcctagccaggaagagatgaccaagaatcaggtgtccctgacatgcctggtgaagggcttctacccttccgatatcgccgtggagtgggagagcaacggccagccagagaacaactacaagaccacccctcctgtgctggactccgacggctccttcttcctgtactccaggctgaccgtggacaagtcccggtggcaggaaggcaacgtcttttcctgctccgtgatgcacgaggccctgcacaaccactacacccagaagtccctgtccctgtctctgggcaag

SEQ ID NO: 83 - DNA, 393 bp, Artificial Sequence
  source: /note=“Description of Artificial Sequence Synthetic K09A-L-11 light chain variable region sequence”
  sig_peptide (1)..(60)
atggaagccccagctcagcttctcttcctcctgctactctggctcccagataccaccggagaaattgtgttgacacagtctccagccaccctgtctttgtctccaggggaaagagccaccctctcctgcagggccagcaaaggtgtcagtacatctggctatagttatttgcactggtatcaacagaaacctggccaggctcccaggctcctcatctatcttgcatcctacctagaatctggcgtcccagccaggttcagtggtagtgggtctgggacagacttcactctcaccatcagcagcctagagcctgaagattttgcagtttattactgtcagcacagcagggaccttccgctcacgttcggcggagggaccaaagtggagatcaaa

SEQ ID NO: 84 - DNA, 393 bp, Artificial Sequence
  source: /note=“Description of Artificial Sequence Synthetic K09A-L-16 light chain variable region sequence”
  sig_peptide (1)..(60)
atggaaaccccagcgcagcttctcttcctcctgctactctggctcccagataccaccggagaaattgtgctgactcagtctccactctccctgcccgtcacccctggagagccggcctccatctcctgcagggccagcaaaggtgtcagtacatctggctatagttatttgcattggtacctccagaagccagggcagtctccacagctcctgatctatcttgcatcctacctagaatctggggtccctgacaggttcagtggcagtggatcaggcacagattttacactgaaaatcagcagagtggaggctgaggatgttggggtttattactgccagcatagtagggaccttccgctcacgtttggccaggggaccaagctggagatcaaa

SEQ ID NO: 85 - DNA, 393 bp, Artificial Sequence
  source: /note=“Description of Artificial Sequence Synthetic K09A-L-17 light chain variable region sequence”
  sig_peptide (1)..(60)
atgaggctccctgctcagctcctggggctgctaatgctctgggtctctggatccagtggggatattgtgatgacccagactccactctccctgcccgtcacccctggagagccggcctccatctcctgcagggccagcaaaggtgtcagtacatctggctatagttatttgcattggtatctgcagaagccagggcagtctccacagctcctgatctatcttgcatcctacctagaatctggagtcccagacaggttcagtggcagtgggtcaggcactgctttcacactgaaaatcagcagggtggaggctgaggatgttggactttattactgccagcatagtagggaccttccgctcacgtttggccaggggaccaagctggagatcaaa

SEQ ID NO: 86 - DNA, 390 bp, Artificial Sequence
  source: /note=“Description of Artificial Sequence Synthetic Codon optimized K09A-L-11 light chain variable region sequence”
  sig_peptide (1)..(57)
atggcccctgtgcagctgctgggcctgctggtgctgttcctgcctgccatgcggtgcgagatcgtgctgacccagtcccctgccaccctgtccctgagccctggcgagcgggctaccctgagctgcagagcctccaagggcgtgtccacctccggctactcctacctgcactggtatcagcagaagccaggccaggcccctcggctgctgatctacctggcctcctacctggagtccggcgtgcctgcccggttctccggctccggaagcggcaccgacttcaccctgaccatctcctccctggagcctgaggacttcgccgtgtactactgccagcactcccgggacctgcctctgacctttggcggcggaacaaaggtggagatcaag

SEQ ID NO: 87 - DNA, 390 bp, Artificial Sequence
  source: /note=“Description of Artificial Sequence Synthetic Codon optimized K09A-L-16 light chain variable region sequence”
  sig_peptide (1)..(57)
atggcccctgtgcagctgctgggcctgctggtgctgttcctgcctgccatgcggtgcgagatcgtgctgacccagtcccctctgtccctgcctgtgacccctggcgagcctgcctccatctcctgccgggcctccaagggcgtgtccacctccggctactcctacctgcactggtatctgcagaagcctggccagtccccccagctgctgatctacctggcctcctacctggagtccggcgtgcctgaccggttctccggctccggcagcggcaccgacttcaccctgaagatctcccgggtggaggccgaggacgtgggcgtgtactactgccagcactcccgggacctgcctctgaccttcggccagggcaccaagctggagatcaag

SEQ ID NO: 88 - DNA, 390 bp, Artificial Sequence
  source: /note=“Description of Artificial Sequence Synthetic Codon optimized K09A-L-17 light chain variable region sequence”
  sig_peptide (1)..(57)
atggcccctgtgcagctgctgggcctgctggtgctgttcctgcctgccatgcggtgcgacatcgtgatgacccagacccctctgtccctgcctgtgacccctggcgagcctgcctccatctcctgccgggcctccaagggcgtgtccacctccggctactcctacctgcactggtatctgcagaagcctggccagtcccctcagctgctgatctacctggcctcctacctggagtccggcgtgcctgaccggttctccggctccggaagcggcaccgcttttaccctgaagatctccagagtggaggccgaggacgtgggcctgtactactgccagcactcccgggacctgcctctgaccttcggccagggcaccaagctggagatcaag

SEQ ID NO: 89 - protein, 139 aa, Artificial Sequence
  source: /note=“Description of Artificial Sequence Synthetic 109A-H heavy chain variable region sequence”
  MISC_FEATURE (1)..(19): /note=“Signal peptide”
MDWTWSILFLVAAPTGAHSQVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSS

SEQ ID NO: 90 - protein, 466 aa, Artificial Sequence
  source: /note=“Description of Artificial Sequence Synthetic 409A-H heavy chain full length sequence”
  MISC_FEATURE (1)..(19): /note=“Signal peptide”
MAVLGLLFCLVTFPSCVLSQVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 91 - protein, 130 aa, Artificial Sequence
  source: /note=“Description of Artificial Sequence Synthetic K09A-L-11 light chain variable region sequence”
  MISC_FEATURE (1)..(19): /note=“Signal peptide”
MAPVQLLGLLVLFLPAMRCEIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIK

SEQ ID NO: 92 - protein, 130 aa, Artificial Sequence
  source: /note=“Description of Artificial Sequence Synthetic K09A-L-16 light chain variable region sequence”
  MISC_FEATURE (1)..(19): /note=“Signal peptide”
MAPVQLLGLLVLFLPAMRCEIVLTQSPLSLPVTPGEPASISCRASKGVSTSGYSYLHWYLQKPGQSPQLLIYLASYLESGVPDRFSGSGSGTDFTLKISRVEAEDVGVYYCQHSRDLPLTFGQGTKLEIK

SEQ ID NO: 93 - protein, 130 aa, Artificial Sequence
  source: /note=“Description of Artificial Sequence Synthetic K09A-L-17 light chain variable region sequence”
  MISC_FEATURE (1)..(19): /note=“Signal peptide”
MAPVQLLGLLVLFLPAMRCDIVMTQTPLSLPVTPGEPASISCRASKGVSTSGYSYLHWYLQKPGQSPQLLIYLASYLESGVPDRFSGSGSGTAFTLKISRVEAEDVGLYYCQHSRDLPLTFGQGTKLEIK

SEQ ID NO: 94 - protein, 469 aa, Artificial Sequence
  source: /note=“Description of Artificial Sequence Synthetic 109A-H heavy chain full length sequence”
  MISC_FEATURE (1)..(19): /note=“Signal peptide”
MAVLGLLFCLVTFPSCVLSQVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 95 - protein, 237 aa, Artificial Sequence
  source: /note=“Description of Artificial Sequence Synthetic K09A-L-11 light chain full length sequence”
  MISC_FEATURE (1)..(19): /note=“Signal peptide”
MAPVQLLGLLVLFLPAMRCEIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 96 - protein, 237 aa, Artificial Sequence
  source: /note=“Description of Artificial Sequence Synthetic K09A-L-16 light chain full length sequence”
  MISC_FEATURE (1)..(19): /note=“Signal peptide”
MAPVQLLGLLVLFLPAMRCEIVLTQSPLSLPVTPGEPASISCRASKGVSTSGYSYLHWYLQKPGQSPQLLIYLASYLESGVPDRFSGSGSGTDFTLKISRVEAEDVGVYYCQHSRDLPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 97 - protein, 237 aa, Artificial Sequence
  source: /note=“Description of Artificial Sequence Synthetic K09A-L-17 light chain full length sequence”
  MISC_FEATURE (1)..(19): /note=“Signal peptide”
MAPVQLLGLLVLFLPAMRCDIVMTQTPLSLPVTPGEPASISCRASKGVSTSGYSYLHWYLQKPGQSPQLLIYLASYLESGVPDRFSGSGSGTAFTLKISRVEAEDVGLYYCQHSRDLPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 98 - protein, 440 aa, Artificial Sequence
  source: /note=“Description of Artificial Sequence Synthetic polypeptide”
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 99 - protein, 214 aa, Artificial Sequence
  source: /note=“Description of Artificial Sequence Synthetic polypeptide”
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 100 - protein, 113 aa, Artificial Sequence [sequence omitted]

SEQ ID NO: 101 - protein, 107 aa, Artificial Sequence [sequence omitted]

SEQ ID NO: 102 - protein, 5 aa, Artificial Sequence
  source: /note=“Description of Artificial Sequence Synthetic peptide”
NSGMH

SEQ ID NO: 103 - protein, 17 aa, Artificial Sequence
  source: /note=“Description of Artificial Sequence Synthetic peptide”
VIWYDGSKRYYADSVKG

SEQ ID NO: 104 - protein, 4 aa, Artificial Sequence
  source: /note=“Description of Artificial Sequence Synthetic peptide”
NDDY

SEQ ID NO: 105 - protein, 11 aa, Artificial Sequence
  source: /note=“Description of Artificial Sequence Synthetic peptide”
RASQSVSSYLA

SEQ ID NO: 106 - protein, 7 aa, Artificial Sequence
  source: /note=“Description of Artificial Sequence Synthetic peptide”
DASNRAT

SEQ ID NO: 107 - protein, 9 aa, Artificial Sequence
  source: /note=“Description of Artificial Sequence Synthetic peptide”
QQSSNWPRT

SEQ ID NO: 108 - protein, 131 aa, Homo sapiens
QVQLVQSGSELKKPGASVKVSCKASGYTFTSYAMNWVRQAPGQGLEWMGWINTNTGNPTYAQGFTGRFVFSLDTSVSTAYLQISSLKAEDTAVYYCARDSRGYSYYDFWSGYFYYYYMDVWGKGTTVTVSS

SEQ ID NO: 109 - protein, 113 aa, Homo sapiens
DIQMTQSPSSLSASVGDRVTITCQASQDISNYLNWYQQKPGKAPKLLIYDASNLETGVPSRFSGSGSGTDFTFTISSLQPEDIATYYCQQYDNLPPWASYTFGQGTKLEIKRT

SEQ ID NO: 110 - protein, 141 aa, Artificial Sequence
  source: /note=“Description of Artificial Sequence Synthetic polypeptide”
MAWVWTLLFLMAAAQSIQAQVQLVQSGSELKKPGASVKVSCKASGYTFTDYSMHWVRQAPGQGLKWMGWINTETGEPTYADDFKGRFVFSLDTSVSTAYLQISSLKAEDTAVYYCANPYYDYVSYYAMDYWGQGTTVTVSS

SEQ ID NO: 111 - protein, 127 aa, Artificial Sequence
  source: /note=“Description of Artificial Sequence Synthetic polypeptide”
MESQIQVFVFVFLWLSGVDGDIQMTQSPSSLSASVGDRVTITCKASQDVSTAVAWYQQKPGKAPKLLIYSASYLYTGVPSRFSGSGSGTDFTFTISSLQPEDIATYYCQQHYSTPRTFGQGTKLEIK

SEQ ID NO: 112 - protein, 136 aa, Homo sapiens
GLSWVFLVAILEGVQCEVQLVESGGGLVQPGGSLRLSCAASGFTFSSYWMSWVRQAPGKGLEWVANIKQDGSEKYYVDSVKGRFTISRDNAKNSLYLQMNSLRAEDTAVYYCARGLTGATDAFDIWGQGTMVTVSS

SEQ ID NO: 113 - protein, 128 aa, Homo sapiens
MEAPAQLLFLLLLWLPDTTGEIVLTQSPATLSLSPGERATLSCRASQSVSSYLAWYQQKPGQAPRLLIYDASNKATGVPARFSGSGSGTDFTLTISSLEPEDFAVYYCQQSSKWPLTFGGGTKVEIKG

SEQ ID NO: 114 - protein, 139 aa, Artificial Sequence
  source: /note=“Description of Artificial Sequence Synthetic polypeptide”
MDFGLSLVFLVLILKSVQCEVQLVESGGGLVQPGGSLRLSCAASEYEFPSHDMSWVRQAPGKGLELVAAINSDGGSTYYPDTMERRFTISRDNAKNSLYLQMNSLRAEDTAVYYCARHYDDYYAWFAYWGQGTMVTVSS

SEQ ID NO: 115 - protein, 131 aa, Artificial Sequence
  source: /note=“Description of Artificial Sequence Synthetic polypeptide”
METDTLLLWVLLLWVPGSTGEIVLTQSPATLSLSPGERATLSCRASKSVSTSGYSYMHWYQQKPGQAPRLLIYLASNLESGVPARFSGSGSGTDFTLTISSLEPEDFAVYYCQHSRELPLTFGGGTKVEIK

(19) United States
(12) Patent Application Publication
(10) Pub. No.: US 2018/0222989 A1
(43) Pub. Date: Aug. 9, 2018

(54) COMBINATION TREATMENTS AND USES AND METHODS THEREOF

(71) Applicant: GLAXOSMITHKLINE INTELLECTUAL PROPERTY DEVELOPMENT LIMITED, Brentford, Middlesex (GB)

(72) Inventors: Axel HOOS, Collegeville, PA (US); Niranjan YANAMANDRA, Collegeville, PA (US)

(73) Assignee: GLAXOSMITHKLINE INTELLECTUAL PROPERTY DEVELOPMENT LIMITED, Brentford, Middlesex (GB)

(21) Appl. No.: 15/749,355

(22) PCT Filed: Aug. 3, 2016
(86) PCT No.: PCT/IB2016/054695

§ 371 Date: Jan. 31, 2018

Related U.S. Application Data
(60) Provisional application No. 62/200,791, filed on Aug. 4, 2015.

(51) Int. Cl. C07K 16/28; A61P 35/00; A61K 45/06; A61K 39/395

(52) U.S. Cl. CPC C07K 16/2878; A61P 35/00; C07K 16/2818; A61K 2039/545; A61K 39/3955; A61K 39/39558; A61K 45/06

(57) ABSTRACT

Disclosed herein are combinations of an OX40 modulator and a PD-1 modulator, pharmaceutical compositions thereof, uses thereof, and methods of treatment comprising administering said combination, including uses in cancer.

### CROSS REFERENCE TO RELATED APPLICATIONS

This application claims priority to U.S. Application Ser. No. 62/200,791, filed on Aug. 4, 2015. The disclosure of the prior application is considered part of (and are incorporated by reference in) the disclosure of this application.

### SEQUENCE LISTING

The instant application contains a Sequence Listing which has been submitted electronically in ASCII format and is hereby incorporated by reference in its entirety. Said ASCII copy, created on Jul. 29, 2016, is named PU65946PCT_SL.txt and is 110,097 bytes in size.

### FIELD OF THE INVENTION

The present invention relates to a method of treating cancer in a mammal and to combinations useful in such treatment. In particular the present invention relates to combinations of anti-OX40 antigen binding proteins (ABPs), including monoclonal antibodies to human OX40 and one or more anti-PD-1 ABPs, including monoclonal antibodies to human PD-1.

### BACKGROUND OF THE INVENTION

Effective treatment of hyperproliferative disorders including cancer is a continuing goal in the oncology field. Generally, cancer results from the deregulation of the normal processes that control cell division, differentiation and apoptotic cell death and is characterized by the proliferation of malignant cells which have the potential for unlimited growth, local expansion and systemic metastasis. Deregulation of normal processes include abnormalities in signal transduction pathways and response to factors which differ from those found in normal cells.

Immunotherapies are one approach to treat hyperproliferative disorders. A major hurdle that scientists and clinicians have encountered in the development of various types of cancer immunotherapies has been to break tolerance to self antigen (cancer) in order to mount a robust anti-tumor response leading to tumor regression. Unlike traditional development of small and large molecule agents that target the tumor, cancer immunotherapies target cells of the immune system that have the potential to generate a memory pool of effector cells to induce more durable effects and minimize recurrences.

OX40 is a costimulatory molecule involved in multiple processes of the immune system. Antigen binding proteins and antibodies that bind OX-40 receptor and modulate OX40 signalling are known in the art and are disclosed as immunotherapy, for example for cancer.

Binding of the PD-1 ligands, PD-L1 and PD-L2, to the PD-1 receptor found on T cells, inhibits T cell proliferation and cytokine production. Upregulation of PD-1 ligands occurs in some tumors and signaling through this pathway can contribute to inhibition of active T-cell immune surveillance of tumors. Antigen binding proteins and antibodies that bind to the PD-1 receptor and block its interaction with PD-L1 and PD-L2 may release PD-1 pathway-mediated inhibition of the immune response, including the anti-tumor immune response.

Enhancing anti-tumor T cell function and inducing T cell proliferation is a powerful and new approach for cancer treatment. Three immune-oncology antibodies (e.g., immuno-modulators) are presently marketed. Anti-CTLA-4 (YERVOY®/ipilimumab) is thought to augment immune responses at the point of T cell priming and anti-PD-1 antibodies (OPDIVO®/nivolumab and KEYTRUDA®/pembrolizumab) are thought to act in the local tumor microenvironment, by relieving an inhibitory checkpoint in tumor specific T cells that have already been primed and activated.

Though there have been many recent advances in the treatment of cancer, there remains a need for more effective and/or enhanced treatment of an individual suffering the effects of cancer. The combinations and methods herein that relate to combining therapeutic approaches for enhancing anti-tumor immunity address this need.

### SUMMARY OF THE INVENTION

The present invention provides methods of treating cancer in a mammal in need thereof comprising administering a therapeutically effective amount of an antigen binding protein that binds OX40 and an antigen binding protein that binds PD-1.

Also provided are pharmaceutical compositions comprising a therapeutically effective amount of an antigen binding protein that binds OX40 and a therapeutically effective amount of an antigen binding protein that binds PD-1. Suitably, kits are provided comprising the pharmaceutical compositions of the invention together with one or more pharmaceutically acceptable carriers.

Methods are provided for reducing tumor size in a human having cancer comprising administering a therapeutically effective amount of an agonist antibody to human OX-40 and a therapeutically effective amount of an antagonist antibody to human PD-1.

In some aspects, the disclosure provides a method of treating cancer in a mammal in need thereof comprising administering to the mammal a therapeutically effective amount of an antigen binding protein that binds OX40 and an antigen binding protein that binds PD-1.

In some embodiments, the cancer is a solid tumor. In some embodiments, the cancer is selected from the group consisting of: melanoma, lung cancer, kidney cancer, breast cancer, head and neck cancer, colon cancer, ovarian cancer, pancreatic cancer, liver cancer, prostate cancer, bladder cancer, and gastric cancer.

In some embodiments, the cancer is a liquid tumor.

In some embodiments, the antigen binding protein that binds OX40 and the antigen binding that binds PD-1 are administered at the same time.

In some embodiments, the antigen binding protein that binds OX40 and the antigen binding protein that binds PD-1 are administered sequentially, in any order.

In some embodiments, the antigen binding protein that binds OX40 and/or the antigen binding protein that binds PD-1 are administered systemically, e.g. intravenously.

In some embodiments, the antigen binding protein that binds OX40 and/or the antigen binding protein that binds PD-1 is administered intratumorally.

In some embodiments, the mammal is human.

In some embodiments, the tumor size of said cancer in said mammal is reduced by more than an additive amount compared with treatment with the antigen binding protein to OX-40 and the antigen binding protein to PD-1 as used as monotherapy.

In some embodiments, the antigen binding protein that binds OX40 binds to human OX40. In some embodiments, the antigen binding protein that binds to PD-1 binds to human PD-1. In some embodiments, the antigen binding protein that binds OX40 and/or the antigen binding protein that binds PD-1 is a humanized monoclonal antibody.

In some embodiments, the antigen binding protein that binds OX40 and/or the antigen binding protein that binds PD-1 is a fully human monoclonal antibody.

In some embodiments, the antigen binding protein that binds OX40 and/or the antigen binding protein that binds PD-1 is an antibody with an IgG1 antibody isotype or variant thereof.

In some embodiments, the antigen binding protein that binds OX40 and/or the antigen binding protein that binds PD-1 is an antibody with an IgG4 antibody isotype or variant thereof.

In some embodiments, the antigen binding protein that binds OX40 is an agonist antibody.

In some embodiments, the antigen binding protein that binds PD-1 is an antagonist antibody.

In some embodiments, the antigen binding protein that binds OX40 comprises: a heavy chain variable region CDR1 comprising an amino acid sequence with at least 90%, 91%, 92%, 93%, 94%, 95%, 96%, 97%, 98%, 99% or 100% sequence identity to the amino acid sequence set forth in SEQ ID NO:1 or 13; a heavy chain variable region CDR2 comprising an amino acid sequence with at least 90%, 91%, 92%, 93%, 94%, 95%, 96%, 97%, 98%, 99% or 100% sequence identity to the amino acid sequence as set forth in SEQ ID NO:2 or 14; and/or a heavy chain variable region CDR3 comprising an amino acid sequence with at least 90%, 91%, 92%, 93%, 94%, 95%, 96%, 97%, 98%, 99% or 100% sequence identity to the amino acid sequence as set forth in SEQ ID NO:3 or 15.

In some embodiments, the antigen binding protein that binds OX40 comprises a light chain variable region CDR1 comprising an amino acid sequence with at least 90%, 91%, 92%, 93%, 94%, 95%, 96%, 97%, 98%, 99% or 100% sequence identity to the amino acid sequence as set forth in SEQ ID NO:7 or 19; a light chain variable region CDR2 comprising an amino acid sequence with at least at least 90%, 91%, 92%, 93%, 94%, 95%, 96%, 97%, 98%, 99% or 100% sequence identity to the amino acid sequence as set forth in SEQ ID NO:8 or 20 and/or a light chain variable region CDR3 comprising an amino acid sequence with at least 90%, 91%, 92%, 93%, 94%, 95%, 96%, 97%, 98%, 99% or 100% sequence identity to the amino acid sequence as set forth in SEQ ID NO:9 or 21.

In some embodiments, the antigen binding protein that binds OX40 comprises: (a) a heavy chain variable region CDR1 comprising the amino acid sequence of SEQ ID NO:1; (b) a heavy chain variable region CDR2 comprising the amino acid sequence of SEQ ID NO:2; (c) a heavy chain variable region CDR3 comprising the amino acid sequence of SEQ ID NO:3;

(d) a light chain variable region CDR1 comprising the amino acid sequence of SEQ ID NO:7; (e) a light chain variable region CDR2 comprising the amino acid sequence of SEQ ID NO:8; and (f) a light chain variable region CDR3 comprising the amino acid sequence of SEQ ID NO:9.

In some embodiments, the antigen binding protein that binds OX40 comprises: (a) a heavy chain variable region CDR1 comprising the amino acid sequence of SEQ ID NO:13; (b) a heavy chain variable region CDR2 comprising the amino acid sequence of SEQ ID NO:14; (c) a heavy chain variable region CDR3 comprising the amino acid sequence of SEQ ID NO:15; (d) a light chain variable region CDR1 comprising the amino acid sequence of SEQ ID NO:19; (e) a light chain variable region CDR2 comprising the amino acid sequence of SEQ ID NO:20; and (f) a light chain variable region CDR3 comprising the amino acid sequence of SEQ ID NO:21.

In some embodiments, the antigen binding protein that binds OX40 comprises a light chain variable region (“VL”) comprising an amino acid sequence with at least 90%, 91%, 92%, 93%, 94%, 95%, 96%, 97%, 98%, 99% or 100% sequence identity to the amino acid sequence as set forth in SEQ ID NO:10, 11, 22 or 23.

In some embodiments, the antigen binding protein that binds OX40 comprises a heavy chain variable region (“VH”) comprising an amino acid sequence with at least 90%, 91%, 92%, 93%, 94%, 95%, 96%, 97%, 98%, 99% or 100% sequence identity to the amino acid sequence as set forth in SEQ ID NO:4, 5, 16 and 17.

In some embodiments, the antigen binding protein that binds OX40 comprises a heavy chain variable region comprising the amino acid sequence set forth in SEQ ID NO:5 and a light chain variable region comprising the amino acid sequence set forth in SEQ ID NO:11.

In some embodiments, the antigen binding protein that binds OX40 comprises a heavy chain variable region comprising the amino acid sequence set forth in SEQ ID NO:17 and a light chain variable region comprising the amino acid sequence set forth in SEQ ID NO:23.

In some embodiments, the antigen binding protein that binds OX40 comprises a light chain variable region comprising the amino acid sequence of SEQ ID NO:11 or 23, or an amino acid sequence with at least 90% sequence identity to the amino acid sequences of SEQ ID NO:11 or 23.

In some embodiments, the antigen binding protein that binds OX40 comprises a heavy chain variable region comprising the amino acid sequence of SEQ ID NO:5 or 17, or an amino acid sequence with at least 90% sequence identity to the amino acid sequences of SEQ ID NO:5 or 17.

In some embodiments, the monoclonal antibody that binds to human OX40 comprises a heavy chain comprising an amino acid sequence with at least 90%, 91%, 92%, 93%, 94%, 95%, 96%, 97%, 98%, 99% or 100% sequence identity to the amino acid sequence as set forth in SEQ ID NO:48 and a light chain comprising an amino acid sequence with at least 90%, 91%, 92%, 93%, 94%, 95%, 96%, 97%, 98%, 99% or 100% sequence identity to the amino acid sequence as set forth in SEQ ID NO:49.

In some embodiments, the antigen binding protein that binds PD-1 is pembrolizumab, or an antibody comprising 90%, 91%, 92%, 93%, 94%, 95%, 96%, 97%, 98%, 99% or 100% sequence identity thereto, e.g., to the HC CDRs, LC CDRs, VH, VL, HC and/or LC thereof.

In some embodiments, the antigen binding protein that binds PD-1 is nivolumab, or an antibody having 90%, 91%, 92%, 93%, 94%, 95%, 96%, 97%, 98%, 99% or 100% sequence identity thereto, e.g., the HC CDRs, LC CDRs, VH, VL, HC and/or LC thereof.

In some embodiments, the mammal has increased survival when treated with a therapeutically effective amount of an antigen binding protein to OX-40 and therapeutically effective amount of an antigen binding protein to PD-1 compared with a mammal who received the antigen binding protein to OX-40 or the antigen binding protein to PD-1 as monotherapy.

In some embodiments, the method further comprises administering at least one anti-neoplastic agent to the mammal in need thereof.

In some aspects, the disclosure provides a pharmaceutical composition or kit comprising a therapeutically effective amount of an antigen binding protein that binds OX40 and a therapeutically effective amount of an antigen binding protein that binds PD-1.

In some embodiments, pharmaceutical composition or kit comprises an antibody comprising an antigen binding protein that binds OX40 comprising a heavy chain variable region CDR1 having an amino acid sequence with at least 90%, 91%, 92%, 93%, 94%, 95%, 96%, 97%, 98%, 99% or 100% sequence identity to the amino acid sequence as set forth in SEQ ID NO:1, a heavy chain variable region CDR2 having an amino acid sequence with at least 90%, 91%, 92%, 93%, 94%, 95%, 96%, 97%, 98%, 99% or 100% sequence identity to the amino acid sequence as set forth in SEQ ID NO:2, a heavy chain variable region CDR3 having an amino acid sequence with at least 90%, 91%, 92%, 93%, 94%, 95%, 96%, 97%, 98%, 99% or 100% sequence identity to the amino acid sequence as set forth in SEQ ID NO:3, a light chain variable region CDR1 having an amino acid sequence with at least 90%, 91%, 92%, 93%, 94%, 95%, 96%, 97%, 98%, 99% or 100% sequence identity to the amino acid sequence as set forth in SEQ ID NO:7, a light chain variable region CDR2 having an amino acid sequence with at least 90%, 91%, 92%, 93%, 94%, 95%, 96%, 97%, 98%, 99% or 100% sequence identity to the amino acid sequence as set forth in SEQ ID NO:8, a light chain variable region CDR3 having an amino acid sequence with at least 90%, 91%, 92%, 93%, 94%, 95%, 96%, 97%, 98%, 99% or 100% sequence identity to the amino acid sequence as set forth in SEQ ID NO:9; and pembrolizumab, or an antibody comprising 90%, 91%, 92%, 93%, 94%, 95%, 96%, 97%, 98%, 99% or 100% sequence identity thereto, e.g., to the HC CDRs, LC CDRs, VH, VL, HC and/or LC thereof.

In some aspects, the disclosure provides a pharmaceutical composition or kit as described herein, comprising an antibody comprising a VH region having a sequence at least with a sequence at least 90%, 91%, 92%, 93%, 94%, 95%, 96%, 97%, 98%, 99% or 100% sequence identity to the amino acid sequence as set forth in SEQ ID NO:4 or 5 and VL having a sequence at least with a sequence at least 90%, 91%, 92%, 93%, 94%, 95%, 96%, 97%, 98%, 99% or 100% sequence identity to the amino acid sequence as set forth in SEQ ID NO:10 or 11, and pembrolizumab, or an antibody comprising 90%, 91%, 92%, 93%, 94%, 95%, 96%, 97%, 98%, 99% or 100% sequence identity thereto, e.g., to the HC CDRs, LC CDRs, VH, VL, HC and/or LC thereof.

In some embodiments, the pharmaceutical composition or kit comprises an antibody comprising an antigen binding protein that binds OX40 comprising a heavy chain variable region CDR1 having an amino acid sequence with at least 90%, 91%, 92%, 93%, 94%, 95%, 96%, 97%, 98%, 99% or 100% sequence identity to the amino acid sequence as set forth in SEQ ID NO:1, a heavy chain variable region CDR2 having an amino acid sequence with at least 90%, 91%, 92%, 93%, 94%, 95%, 96%, 97%, 98%, 99% or 100% sequence identity to the amino acid sequence as set forth in SEQ ID NO:2, a heavy chain variable region CDR3 having an amino acid sequence with at least 90%, 91%, 92%, 93%, 94%, 95%, 96%, 97%, 98%, 99% or 100% sequence identity to the amino acid sequence as set forth in SEQ ID NO:3, a light chain variable region CDR1 having an amino acid sequence with at least 90%, 91%, 92%, 93%, 94%, 95%, 96%, 97%, 98%, 99% or 100% sequence identity to the amino acid sequence as set forth in SEQ ID NO:7, a light chain variable region CDR2 having an amino acid sequence with at least 90%, 91%, 92%, 93%, 94%, 95%, 96%, 97%, 98%, 99% or 100% sequence identity to the amino acid sequence as set forth in SEQ ID NO:8, a light chain variable region CDR3 having an amino acid sequence with at least 90%, 91%, 92%, 93%, 94%, 95%, 96%, 97%, 98%, 99% or 100% sequence identity to the amino acid sequence as set forth in SEQ ID NO:9; and nivolumab, or an antibody comprising 90%, 91%, 92%, 93%, 94%, 95%, 96%, 97%, 98%, 99% or 100% sequence identity thereto, e.g., to the HC CDRs, LC CDRs, VH, VL, HC and/or LC thereof.

In some aspects, the disclosure provides a pharmaceutical composition or kit as described herein, comprising an antibody comprising a VH region having a sequence at least with a sequence at least 90%, 91%, 92%, 93%, 94%, 95%, 96%, 97%, 98%, 99% or 100% sequence identity to the amino acid sequence as set forth in SEQ ID NO:4 or 5 and VL having a sequence at least with a sequence at least 90%, 91%, 92%, 93%, 94%, 95%, 96%, 97%, 98%, 99% or 100% sequence identity to the amino acid sequence as set forth in SEQ ID NO:10 or 11, and nivolumab, or an antibody comprising 90%, 91%, 92%, 93%, 94%, 95%, 96%, 97%, 98%, 99% or 100% sequence identity thereto, e.g., to the HC CDRs, LC CDRs, VH, VL, HC and/or LC thereof.

In some embodiments, the monoclonal antibody that binds to human OX40 comprises a heavy chain comprising an amino acid sequence with at least 90%, 91%, 92%, 93%, 94%, 95%, 96%, 97%, 98%, 99% or 100% sequence identity to the amino acid sequence as set forth in SEQ ID NO:48 and a light chain comprising an amino acid sequence with at least 90%, 91%, 92%, 93%, 94%, 95%, 96%, 97%, 98%, 99% or 100% sequence identity to the amino acid sequence as set forth in SEQ ID NO:49.

In some aspects, the disclosure provides use of a combination or pharmaceutical composition or kit as described herein in the manufacture of a medicament for the treatment of cancer.

In some aspects, the disclosure provides a combination kit comprising a pharmaceutical composition or kit as described herein together with one or more pharmaceutically acceptable carriers.

In some aspects, the disclosure provides a method of reducing tumor size in a human having cancer comprising administering a therapeutically effective amount of an agonist antibody to human OX-40 and a therapeutically effective amount of an antagonist antibody to human PD-1, e.g., as described herein.

In some aspects, the disclosure provides a kit for use in the treatment of cancer comprising:
a therapeutically effective amount of an antigen binding protein that binds OX40 and an antigen binding protein that binds PD-1, and
instructions for use in the treatment of cancer.

In some embodiments, the cancer is a solid tumor. In some embodiments, the cancer is selected from the group consisting of: melanoma, lung cancer, kidney cancer, breast cancer, head and neck cancer, colon cancer, ovarian cancer, pancreatic cancer, liver cancer, prostate cancer, bladder cancer, and gastric cancer.

In some embodiments, the cancer is a liquid tumor.

In some embodiments, the antigen binding protein that binds OX40 binds to human OX40.

In some embodiments, the antigen binding protein that binds to PD-1 binds to human PD-1.

In some embodiments, the antigen binding protein that binds OX40 and/or the antigen binding protein that binds PD-1 is a humanized monoclonal antibody.

In some embodiments, the antigen binding protein that binds OX40 and/or the antigen binding protein that binds PD-1 is a fully human monoclonal antibody.

In some embodiments, the antigen binding protein that binds OX40 and/or the antigen binding protein that binds PD-1 is an antibody with an IgG1 antibody isotype or variant thereof.

In some embodiments, the antigen binding protein that binds OX40 and/or the antigen binding protein that binds PD-1 is an antibody with an IgG4 antibody isotype or variant thereof.

In some embodiments, the antigen binding protein that binds OX40 is an agonist antibody.

In some embodiments, the antigen binding protein that binds PD-1 is an antagonist antibody.

In some embodiments, the antigen binding protein that binds OX40 comprises: a heavy chain variable region CDR1 comprising an amino acid sequence with at least 90%, 91%, 92%, 93%, 94%, 95%, 96%, 97%, 98%, 99% or 100% sequence identity to the amino acid sequence set forth in SEQ ID NO:1 or 13; a heavy chain variable region CDR2 comprising an amino acid sequence with at least 90%, 91%, 92%, 93%, 94%, 95%, 96%, 97%, 98%, 99% or 100% sequence identity to the amino acid sequence as set forth in SEQ ID NO:2 or 14; and/or a heavy chain variable region CDR3 comprising an amino acid sequence with at least 90%, 91%, 92%, 93%, 94%, 95%, 96%, 97%, 98%, 99% or 100% sequence identity to the amino acid sequence as set forth in SEQ ID NO:3 or 15.

In some embodiments, the antigen binding protein that binds OX40 comprises a light chain variable region CDR1 comprising an amino acid sequence with at least 90%, 91%, 92%, 93%, 94%, 95%, 96%, 97%, 98%, 99% or 100% sequence identity to the amino acid sequence as set forth in SEQ ID NO:7 or 19; a light chain variable region CDR2 comprising an amino acid sequence with at least at least 90%, 91%, 92%, 93%, 94%, 95%, 96%, 97%, 98%, 99% or 100% sequence identity to the amino acid sequence as set forth in SEQ ID NO:8 or 20 and/or a light chain variable region CDR3 comprising an amino acid sequence with at least 90%, 91%, 92%, 93%, 94%, 95%, 96%, 97%, 98%, 99% or 100% sequence identity to the amino acid sequence as set forth in SEQ ID NO:9 or 21.

In some embodiments, the antigen binding protein that binds OX40 comprises: (a) a heavy chain variable region CDR1 comprising the amino acid sequence of SEQ ID NO:1; (b) a heavy chain variable region CDR2 comprising the amino acid sequence of SEQ ID NO:2; (c) a heavy chain variable region CDR3 comprising the amino acid sequence of SEQ ID NO:3; (d) a light chain variable region CDR1 comprising the amino acid sequence of SEQ ID NO:7; (e) a light chain variable region CDR2 comprising the amino acid sequence of SEQ ID NO:8; and (f) a light chain variable region CDR3 comprising the amino acid sequence of SEQ ID NO:9.

In some embodiments, the antigen binding protein that binds OX40 comprises: (a) a heavy chain variable region CDR1 comprising the amino acid sequence of SEQ ID NO:13; (b) a heavy chain variable region CDR2 comprising the amino acid sequence of SEQ ID NO:14; (c) a heavy chain variable region CDR3 comprising the amino acid sequence of SEQ ID NO:15; (d) a light chain variable region CDR1 comprising the amino acid sequence of SEQ ID NO:19; (e) a light chain variable region CDR2 comprising the amino acid sequence of SEQ ID NO:20; and (f) a light chain variable region CDR3 comprising the amino acid sequence of SEQ ID NO:21.

In some embodiments, the antigen binding protein that binds OX40 comprises a light chain variable region (“VL”) comprising an amino acid sequence with at least 90%, 91%, 92%, 93%, 94%, 95%, 96%, 97%, 98%, 99% or 100% sequence identity to the amino acid sequence as set forth in SEQ ID NO:10, 11, 22 or 23.

In some embodiments, the antigen binding protein that binds OX40 comprises a heavy chain variable region (“VH”) comprising an amino acid sequence with at least 90%, 91%, 92%, 93%, 94%, 95%, 96%, 97%, 98%, 99% or 100% sequence identity to the amino acid sequence as set forth in SEQ ID NO:4, 5, 16 and 17.

In some embodiments, the antigen binding protein that binds OX40 comprises a heavy chain variable region comprising the amino acid sequence set forth in SEQ ID NO:5 and a light chain variable region comprising the amino acid sequence set forth in SEQ ID NO:11.

In some embodiments, the antigen binding protein that binds OX40 comprises a heavy chain variable region comprising the amino acid sequence set forth in SEQ ID NO:17 and a light chain variable region comprising the amino acid sequence set forth in SEQ ID NO:23.

In some embodiments, the antigen binding protein that binds OX40 comprises a light chain variable region comprising the amino acid sequence of SEQ ID NO:11 or 23, or an amino acid sequence with at least 90% sequence identity to the amino acid sequences of SEQ ID NO:11 or 23.

In some embodiments, the antigen binding protein that binds OX40 comprises a heavy chain variable region comprising the amino acid sequence of SEQ ID NO:5 or 17, or an amino acid sequence with at least 90% sequence identity to the amino acid sequences of SEQ ID NO:5 or 17.

In some embodiments, the monoclonal antibody that binds to human OX40 comprises a heavy chain comprising an amino acid sequence with at least 90%, 91%, 92%, 93%, 94%, 95%, 96%, 97%, 98%, 99% or 100% sequence identity to the amino acid sequence as set forth in SEQ ID NO:48 and a light chain comprising an amino acid sequence with at least 90%, 91%, 92%, 93%, 94%, 95%, 96%, 97%, 98%, 99% or 100% sequence identity to the amino acid sequence as set forth in SEQ ID NO:49.

In some embodiments, the antigen binding protein that binds PD-1 is pembrolizumab, or an antibody comprising 90%, 91%, 92%, 93%, 94%, 95%, 96%, 97%, 98%, 99% or 100% sequence identity thereto, e.g., to the HC CDRs, LC CDRs, VH, VL, HC and/or LC thereof.

In some embodiments, the antigen binding protein that binds PD-1 is nivolumab, or an antibody having 90%, 91%, 92%, 93%, 94%, 95%, 96%, 97%, 98%, 99% or 100% sequence identity thereto, e.g., the HC CDRs, LC CDRs, VH, VL, HC and/or LC thereof.

In some embodiments, the kit further comprises at least one anti-neoplastic agent.

In some aspects, the disclosure provides a therapeutically effective amount of an antigen binding protein that binds OX40 and an antigen binding protein that binds PD-1 for use (e.g., simultaneous or sequential use) in treating cancer in a mammal in need thereof.

In some embodiments, the cancer is a solid tumor. In some embodiments, the cancer is selected from the group consisting of: melanoma, lung cancer, kidney cancer, breast cancer, head and neck cancer, colon cancer, ovarian cancer, pancreatic cancer, liver cancer, prostate cancer, bladder cancer, and gastric cancer.

In some embodiments, the cancer is a liquid tumor.

In some embodiments, the antigen binding protein that binds OX40 and the antigen binding that binds PD-1 are to be administered at the same time.

In some embodiments, the antigen binding protein that binds OX40 and the antigen binding protein that binds PD-1 are to be administered sequentially, in any order.

In some embodiments, the antigen binding protein that binds OX40 and/or the antigen binding protein that binds PD-1 are to be administered systemically, e.g. intravenously.

In some embodiments, the antigen binding protein that binds OX40 and/or the antigen binding protein that binds PD-1 is to be administered intratumorally.

In some embodiments, the mammal is human.

In some embodiments, the tumor size of said cancer in said mammal is reduced by more than an additive amount compared with treatment with the antigen binding protein to OX-40 and the antigen binding protein to PD-1 as used as monotherapy.

In some embodiments, the antigen binding protein that binds OX40 binds to human OX40.

In some embodiments, the antigen binding protein that binds to PD-1 binds to human PD-1.

In some embodiments, the antigen binding protein that binds OX40 and/or the antigen binding protein that binds PD-1 is a humanized monoclonal antibody.

In some embodiments, the antigen binding protein that binds OX40 and/or the antigen binding protein that binds PD-1 is a fully human monoclonal antibody.

In some embodiments, the antigen binding protein that binds OX40 and/or the antigen binding protein that binds PD-1 is an antibody with an IgG1 antibody isotype or variant thereof.

In some embodiments, the antigen binding protein that binds OX40 and/or the antigen binding protein that binds PD-1 is an antibody with an IgG4 antibody isotype or variant thereof.

In some embodiments, the antigen binding protein that binds OX40 is an agonist antibody.

In some embodiments, the antigen binding protein that binds PD-1 is an antagonist antibody.

In some embodiments, the antigen binding protein that binds OX40 comprises: a heavy chain variable region CDR1 comprising an amino acid sequence with at least 90%, 91%, 92%, 93%, 94%, 95%, 96%, 97%, 98%, 99% or 100% sequence identity to the amino acid sequence set forth in SEQ ID NO:1 or 13; a heavy chain variable region CDR2 comprising an amino acid sequence with at least 90%, 91%, 92%, 93%, 94%, 95%, 96%, 97%, 98%, 99% or 100% sequence identity to the amino acid sequence as set forth in SEQ ID NO:2 or 14; and/or a heavy chain variable region CDR3 comprising an amino acid sequence with at least 90%, 91%, 92%, 93%, 94%, 95%, 96%, 97%, 98%, 99% or 100% sequence identity to the amino acid sequence as set forth in SEQ ID NO:3 or 15.

In some embodiments, the antigen binding protein that binds OX40 comprises a light chain variable region CDR1 comprising an amino acid sequence with at least 90%, 91%, 92%, 93%, 94%, 95%, 96%, 97%, 98%, 99% or 100% sequence identity to the amino acid sequence as set forth in SEQ ID NO:7 or 19; a light chain variable region CDR2 comprising an amino acid sequence with at least at least 90%, 91%, 92%, 93%, 94%, 95%, 96%, 97%, 98%, 99% or 100% sequence identity to the amino acid sequence as set forth in SEQ ID NO:8 or 20 and/or a light chain variable region CDR3 comprising an amino acid sequence with at least 90%, 91%, 92%, 93%, 94%, 95%, 96%, 97%, 98%, 99% or 100% sequence identity to the amino acid sequence as set forth in SEQ ID NO:9 or 21.

In some embodiments, the antigen binding protein that binds OX40 comprises: (a) a heavy chain variable region CDR1 comprising the amino acid sequence of SEQ ID NO:1; (b) a heavy chain variable region CDR2 comprising the amino acid sequence of SEQ ID NO:2; (c) a heavy chain variable region CDR3 comprising the amino acid sequence of SEQ ID NO:3; (d) a light chain variable region CDR1 comprising the amino acid sequence of SEQ ID NO:7; (e) a light chain variable region CDR2 comprising the amino acid sequence of SEQ ID NO:8; and (f) a light chain variable region CDR3 comprising the amino acid sequence of SEQ ID NO:9.

In some embodiments, the antigen binding protein that binds OX40 comprises: (a) a heavy chain variable region CDR1 comprising the amino acid sequence of SEQ ID NO:13; (b) a heavy chain variable region CDR2 comprising the amino acid sequence of SEQ ID NO:14; (c) a heavy chain variable region CDR3 comprising the amino acid sequence of SEQ ID NO:15; (d) a light chain variable region CDR1 comprising the amino acid sequence of SEQ ID NO:19; (e) a light chain variable region CDR2 comprising the amino acid sequence of SEQ ID NO:20; and (f) a light chain variable region CDR3 comprising the amino acid sequence of SEQ ID NO:21.

In some embodiments, the antigen binding protein that binds OX40 comprises a light chain variable region (“VL”) comprising an amino acid sequence with at least 90%, 91%, 92%, 93%, 94%, 95%, 96%, 97%, 98%, 99% or 100% sequence identity to the amino acid sequence as set forth in SEQ ID NO:10, 11, 22 or 23.

In some embodiments, the antigen binding protein that binds OX40 comprises a heavy chain variable region (“VH”) comprising an amino acid sequence with at least 90%, 91%, 92%, 93%, 94%, 95%, 96%, 97%, 98%, 99% or 100% sequence identity to the amino acid sequence as set forth in SEQ ID NO:4, 5, 16 and 17.

In some embodiments, the antigen binding protein that binds OX40 comprises a heavy chain variable region comprising the amino acid sequence set forth in SEQ ID NO:5 and a light chain variable region comprising the amino acid sequence set forth in SEQ ID NO:11.

In some embodiments, the antigen binding protein that binds OX40 comprises a heavy chain variable region comprising the amino acid sequence set forth in SEQ ID NO:17 and a light chain variable region comprising the amino acid sequence set forth in SEQ ID NO:23.

In some embodiments, the antigen binding protein that binds OX40 comprises a light chain variable region comprising the amino acid sequence of SEQ ID NO:11 or 23, or an amino acid sequence with at least 90% sequence identity to the amino acid sequences of SEQ ID NO:11 or 23.

In some embodiments, the antigen binding protein that binds OX40 comprises a heavy chain variable region comprising the amino acid sequence of SEQ ID NO:5 or 17, or an amino acid sequence with at least 90% sequence identity to the amino acid sequences of SEQ ID NO:5 or 17.

In some embodiments, the monoclonal antibody that binds to human OX40 comprises a heavy chain comprising an amino acid sequence with at least 90%, 91%, 92%, 93%, 94%, 95%, 96%, 97%, 98%, 99% or 100% sequence identity to the amino acid sequence as set forth in SEQ ID NO:48 and a light chain comprising an amino acid sequence with at least 90%, 91%, 92%, 93%, 94%, 95%, 96%, 97%, 98%, 99% or 100% sequence identity to the amino acid sequence as set forth in SEQ ID NO:49.

In some embodiments, the antigen binding protein that binds PD-1 is pembrolizumab, or an antibody comprising 90%, 91%, 92%, 93%, 94%, 95%, 96%, 97%, 98%, 99% or 100% sequence identity thereto, e.g., the HC CDRs, LC CDRs, VH, VL, HC and/or LC thereof.

In some embodiments, the antigen binding protein that binds PD-1 is nivolumab, or an antibody having 90%, 91%, 92%, 93%, 94%, 95%, 96%, 97%, 98%, 99% or 100% sequence identity thereto, e.g., the HC CDRs, LC CDRs, VH, VL, HC and/or LC thereof.

In some embodiments, the mammal has increased survival when treated with a therapeutically effective amount of an antigen binding protein to OX-40 and therapeutically effective amount of an antigen binding protein to PD-1 compared with a mammal who received the antigen binding protein to OX-40 or the antigen binding protein to PD-1 as monotherapy.

In some embodiments, the antigen binding proteins are for use with at least one anti-neoplastic agent.

In some aspects, the disclosure provides a therapeutically effective amount of an agonist antibody to human OX-40 and a therapeutically effective amount of an antagonist antibody to human PD-1 for use (e.g., simultaneous or sequential use) in reducing tumor size in a human having cancer.

In some aspects, the disclosure provides use (e.g., simultaneous or sequential use) of a therapeutically effective amount of an antigen binding protein that binds OX40 and an antigen binding protein that binds PD-1 for the preparation of a medicament for treating cancer in a mammal in need thereof.

In some embodiments, the cancer is a solid tumor. In some embodiments, the cancer is selected from the group consisting of: melanoma, lung cancer, kidney cancer, breast cancer, head and neck cancer, colon cancer, ovarian cancer, pancreatic cancer, liver cancer, prostate cancer, bladder cancer, and gastric cancer.

In some embodiments, the cancer is a liquid tumor.

In some embodiments, the antigen binding protein that binds OX40 and the antigen binding that binds PD-1 are administered at the same time.

In some embodiments, the antigen binding protein that binds OX40 and the antigen binding protein that binds PD-1 are administered sequentially, in any order.

In some embodiments, the antigen binding protein that binds OX40 and/or the antigen binding protein that binds PD-1 are administered systemically, e.g. intravenously.

In some embodiments, the antigen binding protein that binds OX40 and/or the antigen binding protein that binds PD-1 is administered intratumorally.

In some embodiments, the mammal is human.

In some embodiments, the tumor size of said cancer in said mammal is reduced by more than an additive amount compared with treatment with the antigen binding protein to OX-40 and the antigen binding protein to PD-1 as used as monotherapy.

In some embodiments, the antigen binding protein that binds OX40 binds to human OX40.

In some embodiments, the antigen binding protein that binds to PD-1 binds to human PD-1.

In some embodiments, the antigen binding protein that binds OX40 and/or the antigen binding protein that binds PD-1 is a humanized monoclonal antibody.

In some embodiments, the antigen binding protein that binds OX40 and/or the antigen binding protein that binds PD-1 is a fully human monoclonal antibody.

In some embodiments, the antigen binding protein that binds OX40 and/or the antigen binding protein that binds PD-1 is an antibody with an IgG1 antibody isotype or variant thereof.

In some embodiments, the antigen binding protein that binds OX40 and/or the antigen binding protein that binds PD-1 is an antibody with an IgG4 antibody isotype or variant thereof.

In some embodiments, the antigen binding protein that binds OX40 is an agonist antibody.

In some embodiments, the antigen binding protein that binds PD-1 is an antagonist antibody.

In some embodiments, the antigen binding protein that binds OX40 comprises: a heavy chain variable region CDR1 comprising an amino acid sequence with at least 90%, 91%, 92%, 93%, 94%, 95%, 96%, 97%, 98%, 99% or 100% sequence identity to the amino acid sequence set forth in SEQ ID NO:1 or 13; a heavy chain variable region CDR2 comprising an amino acid sequence with at least 90%, 91%, 92%, 93%, 94%, 95%, 96%, 97%, 98%, 99% or 100% sequence identity to the amino acid sequence as set forth in SEQ ID NO:2 or 14; and/or a heavy chain variable region CDR3 comprising an amino acid sequence with at least 90%, 91%, 92%, 93%, 94%, 95%, 96%, 97%, 98%, 99% or 100% sequence identity to the amino acid sequence as set forth in SEQ ID NO:3 or 15.

In some embodiments, the antigen binding protein that binds OX40 comprises a light chain variable region CDR1 comprising an amino acid sequence with at least 90%, 91%, 92%, 93%, 94%, 95%, 96%, 97%, 98%, 99% or 100% sequence identity to the amino acid sequence as set forth in SEQ ID NO:7 or 19; a light chain variable region CDR2 comprising an amino acid sequence with at least at least 90%, 91%, 92%, 93%, 94%, 95%, 96%, 97%, 98%, 99% or 100% sequence identity to the amino acid sequence as set forth in SEQ ID NO:8 or 20 and/or a light chain variable region CDR3 comprising an amino acid sequence with at least 90%, 91%, 92%, 93%, 94%, 95%, 96%, 97%, 98%, 99% or 100% sequence identity to the amino acid sequence as set forth in SEQ ID NO:9 or 21.

In some embodiments, the antigen binding protein that binds OX40 comprises: (a) a heavy chain variable region CDR1 comprising the amino acid sequence of SEQ ID NO:1; (b) a heavy chain variable region CDR2 comprising the amino acid sequence of SEQ ID NO:2; (c) a heavy chain variable region CDR3 comprising the amino acid sequence of SEQ ID NO:3; (d) a light chain variable region CDR1 comprising the amino acid sequence of SEQ ID NO:7; (e) a light chain variable region CDR2 comprising the amino acid sequence of SEQ ID NO:8; and (f) a light chain variable region CDR3 comprising the amino acid sequence of SEQ ID NO:9.

In some embodiments, the antigen binding protein that binds OX40 comprises: (a) a heavy chain variable region CDR1 comprising the amino acid sequence of SEQ ID NO:13; (b) a heavy chain variable region CDR2 comprising the amino acid sequence of SEQ ID NO:14; (c) a heavy chain variable region CDR3 comprising the amino acid sequence of SEQ ID NO:15; (d) a light chain variable region CDR1 comprising the amino acid sequence of SEQ ID NO:19; (e) a light chain variable region CDR2 comprising the amino acid sequence of SEQ ID NO:20; and (f) a light chain variable region CDR3 comprising the amino acid sequence of SEQ ID NO:21.

In some embodiments, the antigen binding protein that binds OX40 comprises a light chain variable region (“VL”) comprising an amino acid sequence with at least 90%, 91%, 92%, 93%, 94%, 95%, 96%, 97%, 98%, 99% or 100% sequence identity to the amino acid sequence as set forth in SEQ ID NO:10, 11, 22 or 23.

In some embodiments, the antigen binding protein that binds OX40 comprises a heavy chain variable region (“VH”) comprising an amino acid sequence with at least 90%, 91%, 92%, 93%, 94%, 95%, 96%, 97%, 98%, 99% or 100% sequence identity to the amino acid sequence as set forth in SEQ ID NO:4, 5, 16 and 17.

In some embodiments, the antigen binding protein that binds OX40 comprises a heavy chain variable region comprising the amino acid sequence set forth in SEQ ID NO:5 and a light chain variable region comprising the amino acid sequence set forth in SEQ ID NO:11.

In some embodiments, the antigen binding protein that binds OX40 comprises a heavy chain variable region comprising the amino acid sequence set forth in SEQ ID NO:17 and a light chain variable region comprising the amino acid sequence set forth in SEQ ID NO:23.

In some embodiments, the antigen binding protein that binds OX40 comprises a light chain variable region comprising the amino acid sequence of SEQ ID NO:11 or 23, or an amino acid sequence with at least 90% sequence identity to the amino acid sequences of SEQ ID NO:11 or 23.

In some embodiments, the antigen binding protein that binds OX40 comprises a heavy chain variable region comprising the amino acid sequence of SEQ ID NO:5 or 17, or an amino acid sequence with at least 90% sequence identity to the amino acid sequences of SEQ ID NO:5 or 17.

In some embodiments, the monoclonal antibody that binds to human OX40 comprises a heavy chain comprising an amino acid sequence with at least 90%, 91%, 92%, 93%, 94%, 95%, 96%, 97%, 98%, 99% or 100% sequence identity to the amino acid sequence as set forth in SEQ ID NO:48 and a light chain comprising an amino acid sequence with at least 90%, 91%, 92%, 93%, 94%, 95%, 96%, 97%, 98%, 99% or 100% sequence identity to the amino acid sequence as set forth in SEQ ID NO:49.

In some embodiments, the antigen binding protein that binds PD-1 is pembrolizumab, or an antibody comprising 90%, 91%, 92%, 93%, 94%, 95%, 96%, 97%, 98%, 99% or 100% sequence identity thereto, e.g., the HC CDRs, LC CDRs, VH, VL, HC and/or LC thereof.

In some embodiments, the antigen binding protein that binds PD-1 is nivolumab, or an antibody having 90%, 91%, 92%, 93%, 94%, 95%, 96%, 97%, 98%, 99% or 100% sequence identity thereto, e.g., the HC CDRs, LC CDRs, VH, VL, HC and/or LC thereof.

In some embodiments, the mammal has increased survival when treated with a therapeutically effective amount of an antigen binding protein to OX-40 and therapeutically effective amount of an antigen binding protein to PD-1 compared with a mammal who received the antigen binding protein to OX-40 or the antigen binding protein to PD-1 as monotherapy.

In some embodiments, the use further comprises at least one anti-neoplastic agent for administration to the mammal in need thereof.

In some aspects, the disclosure provides use of a therapeutically effective amount of an agonist antibody to human OX-40 and a therapeutically effective amount of an antagonist antibody to human PD-1 for the preparation of a medicament for reducing tumor size in a human having cancer.

In some aspects, the disclosure provides a method for increasing IFNg protein or IFNg mRNA levels (e.g., determined as described herein) in a mammal, the method comprising:

administering to the mammal a therapeutically effective amount of an antigen binding protein that binds OX40 and an antigen binding protein that binds PD-1, wherein the antigen binding protein that binds OX40 and the antigen binding protein that binds PD-1 are as described herein, and, e.g., administered as described herein. E.g., wherein the mammal (e.g., human) has cancer, as described herein. Also provided herein is a therapeutically effective amount of an antigen binding protein that binds OX40 and an antigen binding protein that binds PD-1, both as described herein, for use in increasing IFNg protein or mRNA levels in the mammal. Also provided herein is use of a therapeutically effective amount of an antigen binding protein that binds OX40 and an antigen binding protein that binds PD-1, both as described herein, for the preparation of a medicament for increasing IFNg protein or mRNA levels in the mammal.

In some aspects, the disclosure provides a method for increasing TNF-α protein or TNF-α mRNA levels (e.g., determined as described herein) in a mammal, the method comprising:

administering to the mammal a therapeutically effective amount of an antigen binding protein that binds OX40 and an antigen binding protein that binds PD-1, wherein the antigen binding protein that binds OX40 and the antigen binding protein that binds PD-1 are as described herein, and, e.g., administered as described herein. E.g., wherein the mammal (e.g., human) has cancer, as described herein. Also provided herein is a therapeutically effective amount of an antigen binding protein that binds OX40 and an antigen binding protein that binds PD-1, both as described herein, for use in increasing TNF-α protein or mRNA levels in the mammal. Also provided herein is use of a therapeutically effective amount of an antigen binding protein that binds OX40 and an antigen binding protein that binds PD-1, both as described herein, for the preparation of a medicament for increasing TNF-α protein or mRNA levels in the mammal.

In some aspects, the disclosure provides a method for increasing IL-6 protein or IL-6 mRNA levels (e.g., determined as described herein) in a mammal, the method comprising:

administering to the mammal a therapeutically effective amount of an antigen binding protein that binds OX40 and an antigen binding protein that binds PD-1, wherein the antigen binding protein that binds OX40 and the antigen binding protein that binds PD-1 are as described herein, and, e.g., administered as described herein. E.g., wherein the mammal (e.g., human) has cancer, as described herein. Also provided herein is a therapeutically effective amount of an antigen binding protein that binds OX40 and an antigen binding protein that binds PD-1, both as described herein, for use in increasing IL-6 protein or mRNA levels in the mammal. Also provided herein is use of a therapeutically effective amount of an antigen binding protein that binds OX40 and an antigen binding protein that binds PD-1, both as described herein, for the preparation of a medicament for increasing IL-6 protein or mRNA levels in the mammal.

In some aspects, the disclosure provides a method for increasing CCL5 protein or CCL5 mRNA levels (e.g., determined as described herein) in a mammal, the method comprising:

administering to the mammal a therapeutically effective amount of an antigen binding protein that binds OX40 and an antigen binding protein that binds PD-1, wherein the antigen binding protein that binds OX40 and the antigen binding protein that binds PD-1 are as described herein, and, e.g., administered as described herein. E.g., wherein the mammal (e.g., human) has cancer, as described herein. Also provided herein is a therapeutically effective amount of an antigen binding protein that binds OX40 and an antigen binding protein that binds PD-1, both as described herein, for use in increasing CCL5 protein or mRNA levels in the mammal. Also provided herein is use of a therapeutically effective amount of an antigen binding protein that binds OX40 and an antigen binding protein that binds PD-1, both as described herein, for the preparation of a medicament for increasing CCL5 protein or mRNA levels in the mammal.

In some aspects, the disclosure provides a method for increasing CCL5 protein or CCL5 mRNA levels (e.g., determined as described herein) in a mammal, the method comprising:

administering to the mammal a therapeutically effective amount of an antigen binding protein that binds OX40, wherein the antigen binding protein that binds OX40 is as described herein, and, e.g., administered as described herein. E.g., wherein the mammal (e.g., human) has cancer, as described herein. Also provided herein is a therapeutically effective amount of an antigen binding protein that binds OX40, as described herein, for use in increasing CCL5 protein or mRNA levels in the mammal. Also provided herein is use of a therapeutically effective amount of an antigen binding protein that binds OX40, as described herein, for the preparation of a medicament for increasing CCL5 protein or mRNA levels in the mammal.

In some aspects, the disclosure provides a method for increasing CCR5 protein or CCR5 mRNA levels (e.g., determined as described herein) in a mammal, the method comprising:

administering to the mammal a therapeutically effective amount of an antigen binding protein that binds OX40 and an antigen binding protein that binds PD-1, wherein the antigen binding protein that binds OX40 and the antigen binding protein that binds PD-1 are as described herein, and, e.g., administered as described herein. E.g., wherein the mammal (e.g., human) has cancer, as described herein. Also provided herein is a therapeutically effective amount of an antigen binding protein that binds OX40 and an antigen binding protein that binds PD-1, both as described herein, for use in increasing CCR5 protein or mRNA levels in the mammal. Also provided herein is use of a therapeutically effective amount of an antigen binding protein that binds OX40 and an antigen binding protein that binds PD-1, both as described herein, for the preparation of a medicament for increasing CCR5 protein or mRNA levels in the mammal.

In some aspects, the disclosure provides a method for increasing CCR5 protein or CCR5 mRNA levels (e.g., determined as described herein) in a mammal, the method comprising:

administering to the mammal a therapeutically effective amount of an antigen binding protein that binds OX40, wherein the antigen binding protein that binds OX40 is as described herein, and, e.g., administered as described herein. E.g., wherein the mammal (e.g., human) has cancer, as described herein. Also provided herein is a therapeutically effective amount of an antigen binding protein that binds OX40, as described herein, for use in increasing CCR5 protein or mRNA levels in the mammal. Also provided herein is use of a therapeutically effective amount of an antigen binding protein that binds OX40, as described herein, for the preparation of a medicament for increasing CCR5 protein or mRNA levels in the mammal.

In some aspects, the disclosure provides a method for increasing PD-1 protein or PD-1 mRNA levels (e.g., determined as described herein) in a mammal, the method comprising:

administering to the mammal a therapeutically effective amount of an antigen binding protein that binds OX40 and an antigen binding protein that binds PD-1, wherein the antigen binding protein that binds OX40 and the antigen binding protein that binds PD-1 are as described herein and, e.g., administered as described herein. E.g., wherein the mammal (e.g., human) has cancer, as described herein. Also provided herein is a therapeutically effective amount of an antigen binding protein that binds OX40 and an antigen binding protein that binds PD-1, both as described herein, for use in increasing PD-1 protein or mRNA levels in the mammal. Also provided herein is use of a therapeutically effective amount of an antigen binding protein that binds OX40 and an antigen binding protein that binds PD-1, both as described herein, for the preparation of a medicament for increasing PD-1 protein or mRNA levels in the mammal.

In some aspects, the disclosure provides a method for increasing PD-1 protein or PD-1 mRNA levels (e.g., determined as described herein) in a mammal, the method comprising:

administering to the mammal a therapeutically effective amount of an antigen binding protein that binds OX40, wherein the antigen binding protein that binds OX40 is as described herein and, e.g., administered as described herein. E.g., wherein the mammal (e.g., human) has cancer, as described herein. Also provided herein is a therapeutically effective amount of an antigen binding protein that binds OX40, as described herein, for use in increasing PD-1 protein or mRNA levels in the mammal. Also provided herein is use of a therapeutically effective amount of an antigen binding protein that binds OX40, as described herein, for the preparation of a medicament for increasing PD-1 protein or mRNA levels in the mammal.

In some aspects, the disclosure provides a method for increasing PD-L1 protein or PD-L1 mRNA levels (e.g., determined as described herein) in a mammal, the method comprising:

administering to the mammal a therapeutically effective amount of an antigen binding protein that binds OX40 and an antigen binding protein that binds PD-1, wherein the antigen binding protein that binds OX40 and the antigen binding protein that binds PD-1 are as described herein and, e.g., administered as described herein. E.g., wherein the mammal (e.g., human) has cancer, as described herein. Also provided herein is a therapeutically effective amount of an antigen binding protein that binds OX40 and an antigen binding protein that binds PD-1, both as described herein, for use in increasing PD-L1 protein or mRNA levels in the mammal. Also provided herein is use of a therapeutically effective amount of an antigen binding protein that binds OX40 and an antigen binding protein that binds PD-1, both as described herein, for the preparation of a medicament for increasing PD-L1 protein or mRNA levels in the mammal.

In some aspects, the disclosure provides a method for increasing PD-L1 protein or PD-L1 mRNA levels (e.g., determined as described herein) in a mammal, the method comprising:

administering to the mammal a therapeutically effective amount of an antigen binding protein that binds OX40, wherein the antigen binding protein that binds OX40 is as described herein and, e.g., administered as described herein. E.g., wherein the mammal (e.g., human) has cancer, as described herein. Also provided herein is a therapeutically effective amount of an antigen binding protein that binds OX40, as described herein, for use in increasing PD-L1 protein or mRNA levels in the mammal. Also provided herein is use of a therapeutically effective amount of an antigen binding protein that binds OX40, as described herein, for the preparation of a medicament for increasing PD-L1 protein or mRNA levels in the mammal.

In some aspects, the disclosure provides a method for increasing CD4+ and/or CD8+ T cell proliferation (e.g., as determined by Ki67+ staining) in blood in a mammal, the method comprising:

administering to the mammal a therapeutically effective amount of an antigen binding protein that binds OX40 and an antigen binding protein that binds PD-1, wherein the antigen binding protein that binds OX40 and the antigen binding protein that binds PD-1 are as described herein and, e.g., administered as described herein. E.g., wherein the mammal (e.g., human) has cancer, as described herein. Also provided herein is a therapeutically effective amount of an antigen binding protein that binds OX40 and an antigen binding protein that binds PD-1, both as described herein, for use in increasing CD4+ and/or CD8+ T cell proliferation in blood in the mammal. Also provided herein is use of a therapeutically effective amount of an antigen binding protein that binds OX40 and an antigen binding protein that binds PD-1, both as described herein, for the preparation of a medicament for increasing CD4+ and/or CD8+ T cell proliferation in blood in the mammal.

In some aspects, the disclosure provides a method for increasing CD4+ and/or CD8+ T cell effector memory (e.g., measured as described herein) in blood in a mammal, the method comprising:

administering to the mammal a therapeutically effective amount of an antigen binding protein that binds OX40 and an antigen binding protein that binds PD-1, wherein the antigen binding protein that binds OX40 and the antigen binding protein that binds PD-1 are as described herein and, e.g., administered as described herein. E.g., wherein the mammal (e.g., human) has cancer, as described herein. Also provided herein is a therapeutically effective amount of an antigen binding protein that binds OX40 and an antigen binding protein that binds PD-1, both as described herein, for use in increasing CD4+ and/or CD8+ T cell effector memory in blood in the mammal. Also provided herein is use of a therapeutically effective amount of an antigen binding protein that binds OX40 and an antigen binding protein that binds PD-1, both as described herein, for the preparation of a medicament for increasing CD4+ and/or CD8+ T cell effector memory in blood in the mammal.

In some aspects, the disclosure provides a method for increasing CD8+ T cell activation (e.g., as determined by CD25+ staining) in blood in a mammal, the method comprising:

administering to the mammal a therapeutically effective amount of an antigen binding protein that binds OX40 and an antigen binding protein that binds PD-1, wherein the antigen binding protein that binds OX40 and the antigen binding protein that binds PD-1 are as described herein and, e.g., administered as described herein. E.g., wherein the mammal (e.g., human) has cancer, as described herein. Also provided herein is a therapeutically effective amount of an antigen binding protein that binds OX40 and an antigen binding protein that binds PD-1, both as described herein, for use in increasing CD8+ T cell activation in blood in the mammal. Also provided herein is use of a therapeutically effective amount of an antigen binding protein that binds OX40 and an antigen binding protein that binds PD-1, both as described herein, for the preparation of a medicament for increasing CD8+ T cell activation in blood in the mammal.

In some aspects, the disclosure provides a method for increasing PD-1 expression in CD8+ T cells in blood in a mammal, the method comprising:

administering to the mammal a therapeutically effective amount of an antigen binding protein that binds OX40 and an antigen binding protein that binds PD-1, wherein the antigen binding protein that binds OX40 and the antigen binding protein that binds PD-1 are as described herein and, e.g., administered as described herein. E.g., wherein the mammal (e.g., human) has cancer, as described herein. Also provided herein is a therapeutically effective amount of an antigen binding protein that binds OX40 and an antigen binding protein that binds PD-1, both as described herein, for use in increasing PD-1 expression in CD8+ T cells in blood in the mammal. Also provided herein is use of a therapeutically effective amount of an antigen binding protein that binds OX40 and an antigen binding protein that binds PD-1, both as described herein, for the preparation of a medicament for increasing PD-1 expression in CD8+ T cells in blood in the mammal.

In some aspects, the disclosure provides a method for increasing granzyme B levels in CD8+ T cells (e.g., determined as described herein) in a tumor in a mammal, the method comprising:

administering to the mammal a therapeutically effective amount of an antigen binding protein that binds OX40 and an antigen binding protein that binds PD-1, wherein the antigen binding protein that binds OX40 and the antigen binding protein that binds PD-1 are as described herein and, e.g., administered as described herein. E.g., wherein the mammal (e.g., human) has cancer, as described herein. Also provided herein is a therapeutically effective amount of an antigen binding protein that binds OX40 and an antigen binding protein that binds PD-1, both as described herein, for use in increasing granzyme B levels in CD8+ T cells in a tumor in the mammal. Also provided herein is use of a therapeutically effective amount of an antigen binding protein that binds OX40 and an antigen binding protein that binds PD-1, both as described herein, for the preparation of a medicament for increasing granzyme B levels in CD8+ T cells in a tumor in the mammal.

In some aspects, the disclosure provides a method for increasing CD8:Treg ratio (e.g., as determined by CD25+ staining) in tumor in a mammal, the method comprising:

administering to the mammal a therapeutically effective amount of an antigen binding protein that binds OX40 and an antigen binding protein that binds PD-1, wherein the antigen binding protein that binds OX40 and the antigen binding protein that binds PD-1 are as described herein and, e.g., administered as described herein. E.g., wherein the mammal (e.g., human) has cancer, as described herein. Also provided herein is a therapeutically effective amount of an antigen binding protein that binds OX40 and an antigen binding protein that binds PD-1, both as described herein, for use in increasing CD8:Treg ratio in tumor in the mammal. Also provided herein is use of a therapeutically effective amount of an antigen binding protein that binds OX40 and an antigen binding protein that binds PD-1, both as described herein, for the preparation of a medicament for increasing CD8:Treg ratio in tumor in the mammal.

In some aspects, the disclosure provides a method for increasing clonal expansion of T cells (e.g., determined as described herein) in blood and/or tumor in a mammal, the method comprising:

administering to the mammal a therapeutically effective amount of an antigen binding protein that binds OX40 and an antigen binding protein that binds PD-1, wherein the antigen binding protein that binds OX40 and the antigen binding protein that binds PD-1 are as described herein and, e.g., administered as described herein. E.g., wherein the mammal (e.g., human) has cancer, as described herein. Also provided herein is a therapeutically effective amount of an antigen binding protein that binds OX40 and an antigen binding protein that binds PD-1, both as described herein, for use in increasing clonal expansion of T cells in blood and/or tumor in the mammal. Also provided herein is use of a therapeutically effective amount of an antigen binding protein that binds OX40 and an antigen binding protein that binds PD-1, both as described herein, for the preparation of a medicament for increasing clonal expansion of T cells in blood and/or tumor in the mammal.

In some aspects, the disclosure provides a method for increasing anti-PD-1 mediated clonal expansion of T cells (e.g., determined as described herein) in blood and/or tumor in a mammal, the method comprising:

administering to the mammal a therapeutically effective amount of an antigen binding protein that binds OX40, wherein the antigen binding protein that binds OX40 is as described herein and, e.g., administered as described herein. E.g., wherein the mammal (e.g., human) has cancer, as described herein. Also provided herein is a therapeutically effective amount of an antigen binding protein that binds OX40, described herein, for use in increasing anti-PD-1 mediated clonal expansion of T cells in blood and/or tumor in the mammal. Also provided herein is use of a therapeutically effective amount of an antigen binding protein that binds OX40, as described herein, for the preparation of a medicament for increasing anti-PD-1 mediated clonal expansion of T cells in blood and/or tumor in the mammal.

In some aspects, the disclosure provides a method for increasing anti-OX40 mediated clonal expansion of T cells (e.g., determined as described herein) in blood and/or tumor in a mammal, the method comprising:

administering to the mammal a therapeutically effective amount of an antigen binding protein that binds PD-1, wherein the antigen binding protein that binds PD-1 is as described herein and, e.g., administered as described herein. E.g., wherein the mammal (e.g., human) has cancer, as described herein. Also provided herein is a therapeutically effective amount of an antigen binding protein that binds PD-1, described herein, for use in increasing anti-OX40 mediated clonal expansion of T cells in blood and/or tumor in the mammal. Also provided herein is use of a therapeutically effective amount of an antigen binding protein that binds PD-1, as described herein, for the preparation of a medicament for increasing anti-OX40 mediated clonal expansion of T cells in blood and/or tumor in the mammal.

### BRIEF DESCRIPTION OF THE DRAWINGS

FIGS. 1-12 show sequences of the anti-OX40 ABPs of a combination of the invention, or a method or use thereof, e.g. CDRs and VH and VL sequences.

FIG. 13 is a series of panels showing line graphs showing tumor measurements (mm³) over time with the indicated treatments, as listed in panels a)-k).

FIG. 14 is a line graph showing survival after the indicated treatments.

FIGS. 15A, 15B, and 15C. FIG. 15A is a series of panels of tumor growth curves showing tumor volume (mm³) over time with the indicated treatments. FIG. 15B is a line graph showing percent survival after the indicated treatments. FIG. 15C is a line graph showing tumor measurements (mm³) over time after rechallenge.

FIG. 16A and FIG. 16B are bar graphs showing CCL5 levels (A) and CCR5 levels (B) after the indicated treatments.

FIG. 17A and FIG. 17B are bar graphs showing PD-1 levels (A) and PD-L1 levels (B) after the indicated treatments.

FIG. 18 is a series of three bar graphs showing IFN-g, TNF-α, and IL-6 serum cytokine levels after the indicated treatments on the indicated days of harvest, as measured by MSD.

FIG. 19A and FIG. 19 B are bar graphs showing percent CD4+Ki67+ cells (A) and percent CD8+Ki67+ cells (B) in blood after the indicated treatments on the indicated days of harvest.

FIG. 20A and FIG. 20B are bar graphs showing percent CD4+ effector memory (EM) (CD4+CD62L low CD44 high) cells (A) and percent CD8+EM (CD8+CD62L low CD44 high) cells (B) in blood after the indicated treatments on the indicated days of harvest.

FIG. 21A and FIG. 21B are bar graphs showing percent CD8+CD25+ cells (a) and percent CD8+PD01+ cells (b) in blood after the indicated treatments on the indicated days of harvest.

FIG. 22A and FIG. 22B are bar graphs showing percent CD8+ granzyme B+ cells (A) and CD8:Treg ratio (B) in tumor after the indicated treatments on the indicated days of harvest.

FIG. 23A and FIG. 23B are bar graphs showing number of expanded clones in blood (A) and T cell fraction in tumor (B) after the indicated treatments.

FIG. 24A and FIG. 24B are bar graphs showing clonality post treatment in blood (A) and clonality post treatment in tumor (B) after the indicated treatments.

FIG. 25A and FIG. 25B are bar graphs showing IFN-g levels (A) and TNF-α levels (B) after the indicated treatments.

### DETAILED DESCRIPTION OF THE INVENTION

### Compositions and Combinations

Improved function of the immune system is a goal of immunotherapy for cancer. While not being bound by theory, it is thought that for the immune system to be activated and effectively cause regression or eliminate tumors, there must be efficient cross talk among the various compartments of the immune system as well at the at the tumor bed. The tumoricidal effect is dependent on one or more steps, e.g. the uptake of antigen by immature dendritic cells and presentation of processed antigen via MHC I and II by mature dendritic cells to naïve CD8 (cytotoxic) and CD4 (helper) lymphocytes, respectively, in the draining lymph nodes. Naive T cells express molecules such as CTLA-4 and CD28 that engage with co-stimulatory molecules of the B7 family on antigen presenting cells (APCs) such as dendritic cells. In order to keep T cells in check during immune surveillance, B7 on APCs preferentially binds to CTLA-4, an inhibitory molecule on T lymphocytes. However, upon engagement of the T cell receptor (TCR) with MHC Class I or II receptors via cognate peptide presentation on APCs, the co-stimulatory molecule disengages from CTLA-4 and instead binds to the lower affinity stimulatory molecule CD28, causing T cell activation and proliferation. This expanded population of primed T lymphocytes retains memory of the antigen that was presented to them as they traffic to distant tumor sites. Upon encountering a tumor cell bearing the cognate antigen, they eliminate the tumor via cytolytic mediators such as granzyme B and perforins. This apparently simplistic sequence of events is highly dependent on several cytokines, co-stimulatory molecules and check point modulators to activate and differentiate these primed T lymphocytes to a memory pool of cells that can eliminate the tumor.

Thus, an emerging immunotherapeutic strategy is to target T cell co-stimulatory molecules, e.g. OX40. OX40 (e.g. human OX40 (hOX40) or hOX40R) is a tumor necrosis factor receptor family member that is expressed, among other cells, on activated CD4 and CD8 T cells. One of its functions is in the differentiation and long-term survival of these cells. The ligand for OX40 (OX40L) is expressed by activated antigen-presenting cells. Not wishing to be bound by theory, the anti-OX40 ABPs of a combination of the invention, or a method or use thereof, modulate OX40 and promote growth and/or differentiation of T cells and increase long-term memory T-cell populations, e.g. in overlapping mechanisms as those of OX40L, by “engaging” OX40. Thus, in one embodiment of the ABPs of a combination of the invention, or a method or use thereof, bind and engage OX40. In another embodiment, the anti-OX40 ABPs of a combination of the invention, or a method or use thereof, modulate OX40. In a further embodiment, the ABPs of a combination of the invention, or a method or use thereof, modulate OX40 by mimicking OX40L. In another embodiment, the anti-OX40 ABPs of a combination of the invention, or a method or use thereof, are agonist antibodies. In another embodiment, the anti-OX40 ABPs of a combination of the invention, or a method or use thereof, modulate OX40 and cause proliferation of T cells. In a further embodiment, the anti-OX40 ABPs of a combination of the invention, or a method or use thereof, modulate OX40 and improve, augment, enhance, or increase proliferation of CD4 T cells. In another embodiment, the anti-OX40 ABPs of a combination of the invention, or a method or use thereof, improve, augment, enhance, or increase proliferation of CD8 T cells. In further embodiment, the anti-OX40 ABPs of a combination of the invention, or a method or use thereof, improve, augment, enhance, or increase proliferation of both CD4 and CD8 T cells.

In another embodiment, the anti-OX40 ABPs of a combination of the invention, or a method or use thereof, enhance T cell function, e.g. of CD4 or CD8 T cells, or both CD4 and CD8 T cells. In a further embodiment, the anti-OX40 ABPs of a combination of the invention, or a method or use thereof, enhance effector T cell function. In another embodiment, the anti-OX40 ABPs of a combination of the invention, or a method or use thereof, improve, augment, enhance, or increase long-term survival of CD8 T cells. In further embodiments, any of the preceding effects occur in a tumor microenvironment.

Not being bound by theory, of equal importance is the blockade of a potentially robust immunosuppressive response at the tumor site by mediators produced both by T regulatory cells (Tregs) as well as the tumor itself (e.g. Transforming Growth Factor (TGF-B) and interleukin-10 (IL-10)). Not wishing to be bound by theory, a key immune pathogenesis of cancer can be the involvement of Tregs that are found in tumor beds and sites of inflammation. In general, Treg cells occur naturally in circulation and help the immune system to return to a quiet, although vigilant state, after encountering and eliminating external pathogens. They help to maintain tolerance to self antigens and are naturally suppressive in function. They are phenotypically characterized as CD4+, CD25+, FOXP3+ cells. Not wishing to be bound by theory, but in order to break tolerance to effectively treat certain cancers, one mode of therapy is to eliminate Tregs preferentially at tumor sites. Targeting and eliminating Tregs leading to an antitumor response has been more successful in tumors that are immunogenic compared to those that are poorly immunogenic. Many tumors secrete cytokines, e.g. TGF-B that may hamper the immune response by causing precursor CD4+25+ cells to acquire the FOXP3+ phenotype and function as Tregs.

“Modulate” as used herein, for example with regard to a receptor or other target means to change any natural or existing function of the receptor, for example it means affecting binding of natural or artificial ligands to the receptor or target; it includes initiating any partial or full conformational changes or signaling through the receptor or target, and also includes preventing partial or full binding of the receptor or target with its natural or artificial ligands. Also included in the case of membrane bound receptors or targets are any changes in the way the receptor or target interacts with other proteins or molecules in the membrane or change in any localization (or co-localization with other molecules) within membrane compartments as compared to its natural or unchanged state. Modulators are therefore compounds or ligands or molecules that modulate a target or receptor. Modulate includes agonizing, e.g. signaling, as well as antagonizing, or blocking signaling or interactions with a ligand or compound or molecule that happen in the unchanged or unmodulated state. Thus, modulators may be agonists or antagonists. Further, one of skill in the art will recognize that not all modulators will be have absolute selectivity for one target or receptor, but are still considered a modulator for that target or receptor; for example, a modulator may also engage multiple targets.

As used herein the term “agonist” refers to an antigen binding protein including but not limited to an antibody, which upon contact with a co-signalling receptor causes one or more of the following (1) stimulates or activates the receptor, (2) enhances, increases or promotes, induces or prolongs an activity, function or presence of the receptor (3) mimics one or more functions of a natural ligand or molecule that interacts with a target or receptor and includes initiating one or more signaling events through the receptor, mimicking one or more functions of a natural ligand, or initiating one or more partial or full conformational changes that are seen in known functioning or signaling through the receptor and/or (4) enhances, increases, promotes or induces the expression of the receptor. Agonist activity can be measured in vitro by various assays know in the art such as, but not limited to, measurement of cell signalling, cell proliferation, immune cell activation markers, cytokine production. Agonist activity can also be measured in vivo by various assays that measure surrogate end points such as, but not limited to the measurement of T cell proliferation or cytokine production.

As used herein the term “antagonist” refers to an antigen binding protein including but not limited to an antibody, which upon contact with a co-signalling receptor causes one or more of the following (1) attenuates, blocks or inactivates the receptor and/or blocks activation of a receptor by its natural ligand, (2) reduces, decreases or shortens the activity, function or presence of the receptor and/or (3) reduces, decrease, abrogates the expression of the receptor. Antagonist activity can be measured in vitro by various assays know in the art such as, but not limited to, measurement of an increase or decrease in cell signalling, cell proliferation, immune cell activation markers, cytokine production. Antagonist activity can also be measured in vivo by various assays that measure surrogate end points such as, but not limited to the measurement of T cell proliferation or cytokine production.

Thus, in one embodiment, an agonist anti-OX40 ABP inhibits the suppressive effect of Treg cells on other T cells, e.g. within the tumor environment.

Accumulating evidence suggests that the ratio of Tregs to T effector cells in the tumor correlates with anti tumor response. Therefore, in one embodiment, the OX40 ABPs (anti-OX40 ABPs) of a combination of the invention, or a method or use thereof, modulate OX40 to augment T effector number and function and inhibit Treg function.

Enhancing, augmenting, improving, increasing, and otherwise changing the anti-tumor effect of OX40 is an object of a combination of the invention, or a method or use thereof.

Described herein are combinations of an anti-OX40 ABP of a combination of the invention, or a method or use thereof, and another compound, such as a PD-1 modulator (e.g. anti-PD-1 ABP) described herein.

Thus, as used herein the term “combination of the invention” refers to a combination comprising an anti-OX40 ABP, suitably an agonist anti-OX40 ABP, and an anti-PD-1 ABP, suitably an antagonist anti-PD-1 ABP, each of which may be administered separately or simultaneously as described herein.

As used herein, the terms “cancer,” “neoplasm,” and “tumor,” are used interchangeably and in either the singular or plural form, refer to cells that have undergone a malignant transformation or undergone cellular changes that result in aberrant or unregulated growth or hyperproliferation Such changes or malignant transformations usually make such cells pathological to the host organism, thus precancers or precancerous cells that are or could become pathological and require or could benefit from intervention are also intended to be included. Primary cancer cells (that is, cells obtained from near the site of malignant transformation) can be readily distinguished from non-cancerous cells by well-established techniques, particularly histological examination. The definition of a cancer cell, as used herein, includes not only a primary cancer cell, but any cell derived from a cancer cell ancestor. This includes metastasized cancer cells, and in vitro cultures and cell lines derived from cancer cells. When referring to a type of cancer that normally manifests as a solid tumor, a “clinically detectable” tumor is one that is detectable on the basis of tumor mass; e.g., by procedures such as CAT scan, MR imaging, X-ray, ultrasound or palpation, and/or which is detectable because of the expression of one or more cancer-specific antigens in a sample obtainable from a patient. In other words, the terms herein include cells, neoplasms, cancers, and tumors of any stage, including what a clinician refers to as precancer, tumors, in situ growths, as well as late stage metastatic growths, Tumors may be hematopoietic tumor, for example, tumors of blood cells or the like, meaning liquid tumors. Specific examples of clinical conditions based on such a tumor include leukemia such as chronic myelocytic leukemia or acute myelocytic leukemia; myeloma such as multiple myeloma; lymphoma and the like.

As used herein the term “agent” is understood to mean a substance that produces a desired effect in a tissue, system, animal, mammal, human, or other subject. Accordingly, the term “anti-neoplastic agent” is understood to mean a substance producing an anti-neoplastic effect in a tissue, system, animal, mammal, human, or other subject. It is also to be understood that an “agent” may be a single compound or a combination or composition of two or more compounds.

By the term “treating” and derivatives thereof as used herein, is meant therapeutic therapy. In reference to a particular condition, treating means: (1) to ameliorate the condition or one or more of the biological manifestations of the condition; (2) to interfere with (a) one or more points in the biological cascade that leads to or is responsible for the condition or (b) one or more of the biological manifestations of the condition; (3) to alleviate one or more of the symptoms, effects or side effects associated with the condition or one or more of the symptoms, effects or side effects associated with the condition or treatment thereof; (4) to slow the progression of the condition or one or more of the biological manifestations of the condition and/or (5) to cure said condition or one or more of the biological manifestations of the condition by eliminating or reducing to undetectable levels one or more of the biological manifestations of the condition for a period of time considered to be a state of remission for that manifestation without additional treatment over the period of remission. One skilled in the art will understand the duration of time considered to be remission for a particular disease or condition. Prophylactic therapy is also contemplated thereby. The skilled artisan will appreciate that “prevention” is not an absolute term. In medicine, “prevention” is understood to refer to the prophylactic administration of a drug to substantially diminish the likelihood or severity of a condition or biological manifestation thereof, or to delay the onset of such condition or biological manifestation thereof. Prophylactic therapy is appropriate, for example, when a subject is considered at high risk for developing cancer, such as when a subject has a strong family history of cancer or when a subject has been exposed to a carcinogen.

As used herein, “prevention” is understood to refer to the prophylactic administration of a drug to substantially diminish the likelihood or severity of a condition or biological manifestation thereof, or to delay the onset of such condition or biological manifestation thereof. The skilled artisan will appreciate that “prevention” is not an absolute term. Prophylactic therapy is appropriate, for example, when a subject is considered at high risk for developing cancer, such as when a subject has a strong family history of cancer or when a subject has been exposed to a carcinogen.

As used herein, the term “effective amount” means that amount of a drug or pharmaceutical agent that will elicit the biological or medical response of a tissue, system, animal or human that is being sought, for instance, by a researcher or clinician. Furthermore, the term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

The administration of a therapeutically effective amount of the combinations of the invention (or therapeutically effective amounts of each of the components of the combination) are advantageous over the individual component compounds in that the combinations provide one or more of the following improved properties when compared to the individual administration of a therapeutically effective amount of a component compound: i) a greater anticancer effect than the most active single agent, ii) synergistic or highly synergistic anticancer activity, iii) a dosing protocol that provides enhanced anticancer activity with reduced side effect profile, iv) a reduction in the toxic effect profile, v) an increase in the therapeutic window, or vi) an increase in the bioavailability of one or both of the component compounds.

The invention further provides pharmaceutical compositions, which include one or more of the components herein, and one or more pharmaceutically acceptable carriers, diluents, or excipients. The combination of the invention may comprise two pharmaceutical compositions, one comprising an anti-OX40 ABP of the invention, suitably an agonist anti-OX40 ABP, and the other comprising an anti-PD-1 ABP, suitably an antagonist anti-PD-1 ABP, each of which may have the same or different carriers, diluents or excipients. The carrier(s), diluent(s) or excipient(s) must be acceptable in the sense of being compatible with the other ingredients of the formulation, capable of pharmaceutical formulation, and not deleterious to the recipient thereof.

The components of the combination of the invention, and pharmaceutical compositions comprising such components may be administered in any order, and in different routes; the components and pharmaceutical compositions comprising the same may be administered simultaneously.

In accordance with another aspect of the invention there is also provided a process for the preparation of a pharmaceutical composition including admixing a component of the combination of the invention and one or more pharmaceutically acceptable carriers, diluents or excipients.

The components of the invention may be administered by any appropriate route. For some components, suitable routes include oral, rectal, nasal, topical (including buccal and sublingual), vaginal, and parenteral (including subcutaneous, intramuscular, intraveneous, intradermal, intrathecal, and epidural). It will be appreciated that the preferred route may vary with, for example, the condition of the recipient of the combination and the cancer to be treated. It will also be appreciated that each of the agents administered may be administered by the same or different routes and that the components may be compounded together or in separate pharmaceutical compositions.

In one embodiment, one or more components of a combination of the invention are administered intravenously. In another embodiment, one or more components of a combination of the invention are administered intratumorally. In another embodiment, one or more components of a combination of the invention are administered systemically, e.g. intravenously, and one or more other components of a combination of the invention are administered intratumorally. In another embodiment, all of the components of a combination of the invention are administered systemically, e.g. intravenously. In an alternative embodiment, all of the components of the combination of the invention are administered intratumorally. In any of the embodiments, e.g. in this paragraph, the components of the invention are administered as one or more pharmaceutical compositions.

Antigen Binding Proteins that Bind OX40

“Antigen Binding Protein (ABP)” means a protein that binds an antigen, including antibodies or engineered molecules that function in similar ways to antibodies. Such alternative antibody formats include triabody, tetrabody, miniantibody, and a minibody. Also included are alternative scaffolds in which the one or more CDRs of any molecules in accordance with the disclosure can be arranged onto a suitable non-immunoglobulin protein scaffold or skeleton, such as an affibody, a SpA scaffold, an LDL receptor class A domain, an avimer (see, e.g., U.S. Patent Application Publication Nos. 2005/0053973, 2005/0089932, 2005/0164301) or an EGF domain. An ABP also includes antigen binding fragments of such antibodies or other molecules. Further, an ABP of a combination of the invention, or a method or use thereof, may comprise the VH regions formatted into a full length antibody, a (Fab′)2 fragment, a Fab fragment, a bi-specific or biparatopic molecule or equivalent thereof (such as scFV, bi- tri- or tetra-bodies, Tandabs etc.), when paired with an appropriate light chain. The ABP may comprise an antibody that is an IgG1, IgG2, IgG3, or IgG4; or IgM; IgA, IgE or IgD or a modified variant thereof. The constant domain of the antibody heavy chain may be selected accordingly. The light chain constant domain may be a kappa or lambda constant domain. The ABP may also be a chimeric antibody of the type described in WO86/01533 which comprises an antigen binding region and a non-immunoglobulin region.

Thus, herein an anti-OX40 ABP of a combination, or a method or use thereof, of the invention or protein is one that binds OX40, and in preferred embodiments does one or more of the following: modulate signaling through OX40, modulates the function of OX40, agonize OX40 signalling, stimulate OX40 function, or co-stimulate OX40 signaling. One of skill in the art would readily recognize a variety of well known assays to establish such functions.

The term “antibody” as used herein refers to molecules with an antigen binding domain, and optionally an immunoglobulin-like domain or fragment thereof and includes monoclonal (for example IgG, IgM, IgA, IgD or IgE and modified variants thereof), recombinant, polyclonal, chimeric, humanized, biparatopic, bispecific and heteroconjugate antibodies, or a closed conformation multispecific antibody. An “antibody” included xenogeneic, allogeneic, syngeneic, or other modified forms thereof. An antibody may be isolated or purified. An antibody may also be recombinant, i.e. produced by recombinant means; for example, an antibody that is 90% identical to a reference antibody may be generated by mutagenesis of certain residues using recombinant molecular biology techniques known in the art. Thus, the antibodies of the present invention may comprise heavy chain variable regions and light chain variable regions of a combination of the invention, or a method or use thereof, which may be formatted into the structure of a natural antibody or formatted into a full length recombinant antibody, a (Fab′)2 fragment, a Fab fragment, a bi-specific or biparatopic molecule or equivalent thereof (such as scFV, bi- tri- or tetra-bodies, Tandabs etc.), when paired with an appropriate light chain. The antibody may be an IgG1, IgG2, IgG3, or IgG4 or a modified variant thereof. The constant domain of the antibody heavy chain may be selected accordingly. The light chain constant domain may be a kappa or lambda constant domain. The antibody may also be a chimeric antibody of the type described in WO86/01533 which comprises an antigen binding region and a non-immunoglobulin region.

One of skill in the art will recognize that the anti-OX40 ABPs of a combination herein, or method or use thereof, of the invention bind an epitope of OX40; likewise an anti-PD-1 ABP of a combination herein, or a method or use thereof, of the invention binds an epitope of PD-1. The epitope of an ABP is the region of its antigen to which the ABP binds. Two ABPs bind to the same or overlapping epitope if each competitively inhibits (blocks) binding of the other to the antigen. That is, a 1×, 5×, 10×, 20× or 100× excess of one antibody inhibits binding of the other by at least 50% but preferably 75%, 90% or even 99% as measured in a competitive binding assay compared to a control lacking the competing antibody (see, e.g., Junghans et al., Cancer Res. 50:1495, 1990, which is incorporated herein by reference). Alternatively, two antibodies have the same epitope if essentially all amino acid mutations in the antigen that reduce or eliminate binding of one antibody reduce or eliminate binding of the other. Also the same epitope may include “overlapping epitopes” e.g. if some amino acid mutations that reduce or eliminate binding of one antibody reduce or eliminate binding of the other.

The strength of binding may be important in dosing and administration of an ABP of the combination, or method or use thereof, of the invention. In one embodiment, the ABP of the invention binds its target (e.g. OX40 or PD-1) with high affinity. For example, when measured by Biacore, the antibody binds to OX40, preferably human OX40, with an affinity of 1-1000 nM or 500 nM or less or an affinity of 200 nM or less or an affinity of 100 nM or less or an affinity of 50 nM or less or an affinity of 500 pM or less or an affinity of 400 pM or less, or 300 pM or less. In a further aspect the antibody binds to OX40, preferably human OX40, when measured by Biacore of between about 50 nM and about 200 nM or between about 50 nM and about 150 nM. In one aspect of the present invention the antibody binds OX40, preferably human OX40, with an affinity of less than 100 nM.

In a further embodiment, binding is measured by Biacore. Affinity is the strength of binding of one molecule, e.g. an antibody of a combination of the invention, or a method or use thereof, to another, e.g. its target antigen, at a single binding site. The binding affinity of an antibody to its target may be determined by equilibrium methods (e.g. enzyme-linked immunoabsorbent assay (ELISA) or radioimmunoassay (RIA)), or kinetics (e.g. BIACORE analysis). For example, the Biacore methods known in the art may be used to measure binding affinity.

Avidity is the sum total of the strength of binding of two molecules to one another at multiple sites, e.g. taking into account the valency of the interaction.

In an aspect, the equilibrium dissociation constant (KD) of the ABP of a combination of the invention, or a method or use thereof, and OX40, preferably human OX40, interaction is 100 nM or less, 10 nM or less, 2 nM or less or 1 nM or less. Alternatively the KD may be between 5 and 10 nM; or between 1 and 2 nM. The KD may be between 1 pM and 500 pM; or between 500 pM and 1 nM. A skilled person will appreciate that the smaller the KD numerical value, the stronger the binding. The reciprocal of KD (i.e. 1/KD) is the equilibrium association constant (KA) having units M−1. A skilled person will appreciate that the larger the KA numerical value, the stronger the binding.

The dissociation rate constant (kd) or “off-rate” describes the stability of the complex of the ABP on one hand and OX40, preferably human OX40 on the other hand, i.e. the fraction of complexes that decay per second. For example, a kd of 0.01 s−1 equates to 1% of the complexes decaying per second. In an embodiment, the dissociation rate constant (kd) is 1×10−3 s−1 or less, 1×10−4 s−1 or less, 1×10−5 s−1 or less, or 1×10−6 s−1 or less. The kd may be between 1×10−5 s−1 and 1×10−4 s−1; or between 1×10−4 s−1 and 1×10−3 s−1.

Competition between an anti-OX40 ABP of a combination of the invention, or a method or use thereof, and a reference antibody, e.g. for binding OX40, an epitope of OX40, or a fragment of the OX40, may be determined by competition ELISA, FMAT or Biacore. In one aspect, the competition assay is carried out by Biacore. There are several possible reasons for this competition: the two proteins may bind to the same or overlapping epitopes, there may be steric inhibition of binding, or binding of the first protein may induce a conformational change in the antigen that prevents or reduces binding of the second protein.

“Binding fragments” as used herein means a portion or fragment of the ABPs of a combination of the invention, or a method or use thereof, that include the antigen-binding site and are capable of binding OX40 as defined herein, e.g. but not limited to capable of binding to the same epitope of the parent or full length antibody.

Functional fragments of the ABPs of a combination of the invention, or a method or use thereof, are contemplated herein.

Thus, “binding fragments” and “functional fragments” may be an Fab and F(ab′)2 fragments which lack the Fc fragment of an intact antibody, clear more rapidly from the circulation, and may have less non-specific tissue binding than an intact antibody (Wahl et al., J. Nuc. Med. 24:316-325 (1983)). Also included are Fv fragments (Hochman, J. et al. (1973) Biochemistry 12:1130-1135; Sharon, J. et al. (1976) Biochemistry 15:1591-1594). These various fragments are produced using conventional techniques such as protease cleavage or chemical cleavage (see, e.g., Rousseaux et al., Meth. Enzymol., 121:663-69 (1986)).

“Functional fragments” as used herein means a portion or fragment of the ABPs of a combination of the invention, or a method or use thereof, that include the antigen-binding site and are capable of binding the same target as the parent ABP, e.g. but not limited to binding the same epitope, and that also retain one or more modulating or other functions described herein or known in the art.

As the ABPs of the present invention may comprise heavy chain variable regions and light chain variable regions of a combination of the invention, or a method or use thereof, which may be formatted into the structure of a natural antibody, a functional fragment is one that retains binding or one or more functions of the full length ABP as described herein. A binding fragment of an ABP of a combination of the invention, or a method or use thereof, may therefore comprise the VL or VH regions, a (Fab′)2 fragment, a Fab fragment, a fragment of a bi-specific or biparatopic molecule or equivalent thereof (such as scFV, bi- tri- or tetra-bodies, Tandabs etc.), when paired with an appropriate light chain.

The term “CDR” as used herein, refers to the complementarity determining region amino acid sequences of an antigen binding protein. These are the hypervariable regions of immunoglobulin heavy and light chains. There are three heavy chain and three light chain CDRs (or CDR regions) in the variable portion of an immunoglobulin.

It will be apparent to those skilled in the art that there are various numbering conventions for CDR sequences; Chothia (Chothia et al. (1989) Nature 342: 877-883), Kabat (Kabat et al., Sequences of Proteins of Immunological Interest, 4th Ed., U.S. Department of Health and Human Services, National Institutes of Health (1987)), AbM (University of Bath) and Contact (University College London). The minimum overlapping region using at least two of the Kabat, Chothia, AbM and contact methods can be determined to provide the “minimum binding unit”. The minimum binding unit may be a subportion of a CDR. The structure and protein folding of the antibody may mean that other residues are considered part of the CDR sequence and would be understood to be so by a skilled person. It is noted that some of the CDR definitions may vary depending on the individual publication used.

Unless otherwise stated and/or in absence of a specifically identified sequence, references herein to “CDR”, “CDRL1” (or “LC CDR1”), “CDRL2” (or “LC CDR2”), “CDRL3” (or “LC CDR3”), “CDRH1” (or “HC CDR1”), “CDRH2” (or “HC CDR2”), “CDRH3” (or “HC CDR3”) refer to amino acid sequences numbered according to any of the known conventions; alternatively, the CDRs are referred to as “CDR1,” “CDR2,” “CDR3” of the variable light chain and “CDR1,” “CDR2,” and “CDR3” of the variable heavy chain. In particular embodiments, the numbering convention is the Kabat convention.

The term “CDR variant” as used herein, refers to a CDR that has been modified by at least one, for example 1, 2 or 3, amino acid substitution(s), deletion(s) or addition(s), wherein the modified antigen binding protein comprising the CDR variant substantially retains the biological characteristics of the antigen binding protein pre-modification. It will be appreciated that each CDR that can be modified may be modified alone or in combination with another CDR. In one aspect, the modification is a substitution, particularly a conservative substitution, for example as shown in Table 1.

TABLE 1

| Side chain | Members |
|---|---|
| Hydrophobic | Met, Ala, Val, Leu, Ile |
| Neutral hydrophilic | Cys, Ser, Thr |
| Acidic | Asp, Glu |
| Basic | Asn, Gln, His, Lys, Arg |
| Residues that influence chain orientation | Gly, Pro |
| Aromatic | Trp, Tyr, Phe |

For example, in a variant CDR, the amino acid residues of the minimum binding unit may remain the same, but the flanking residues that comprise the CDR as part of the Kabat or

Chothia definition(s) may be substituted with a conservative amino acid residue.

Such antigen binding proteins comprising modified CDRs or minimum binding units as described above may be referred to herein as “functional CDR variants” or “functional binding unit variants”.

The antibody may be of any species, or modified to be suitable to administer to a cross species. For example the CDRs from a mouse antibody may be humanized for administration to humans. In any embodiment, the antigen binding protein is optionally a humanized antibody.

A “humanized antibody” refers to a type of engineered antibody having its CDRs derived from a non-human donor immunoglobulin, the remaining immunoglobulin-derived parts of the molecule being derived from one (or more) human immunoglobulin(s). In addition, framework support residues may be altered to preserve binding affinity (see, e.g., Queen et al., Proc. Natl Acad Sci USA, 86:10029-10032 (1989), Hodgson et al., Bio/Technology, 9:421 (1991)). A suitable human acceptor antibody may be one selected from a conventional database, e.g., the KABAT® database, Los Alamos database, and Swiss Protein database, by homology to the nucleotide and amino acid sequences of the donor antibody. A human antibody characterized by a homology to the framework regions of the donor antibody (on an amino acid basis) may be suitable to provide a heavy chain constant region and/or a heavy chain variable framework region for insertion of the donor CDRs. A suitable acceptor antibody capable of donating light chain constant or variable framework regions may be selected in a similar manner. It should be noted that the acceptor antibody heavy and light chains are not required to originate from the same acceptor antibody. The prior art describes several ways of producing such humanised antibodies—see for example EP-A-0239400 and EP-A-054951.

In yet a further embodiment, the humanized antibody has a human antibody constant region that is an IgG. In another embodiment, the IgG is a sequence as disclosed in any of the above references or patent publications.

For nucleotide and amino acid sequences, the term “identical” or “identity” indicates the degree of identity between two nucleic acid or two amino acid sequences when optimally aligned and compared with appropriate insertions or deletions.

The percent sequence identity between two sequences is a function of the number of identical positions shared by the sequences (i.e., % identity=number of identical positions/total number of positions multiplied by 100), taking into account the number of gaps, and the length of each gap, which need to be introduced for optimal alignment of the two sequences. The comparison of sequences and determination of percent identity between two sequences can be accomplished using a mathematical algorithm, as described below.

Percent identity between a query nucleic acid sequence and a subject nucleic acid sequence is the “Identities” value, expressed as a percentage, which is calculated by the BLASTN algorithm when a subject nucleic acid sequence has 100% query coverage with a query nucleic acid sequence after a pair-wise BLASTN alignment is performed. Such pair-wise BLASTN alignments between a query nucleic acid sequence and a subject nucleic acid sequence are performed by using the default settings of the BLASTN algorithm available on the National Center for Biotechnology Institute's website with the filter for low complexity regions turned off. Importantly, a query nucleic acid sequence may be described by a nucleic acid sequence identified in one or more claims herein.

Percent identity between a query amino acid sequence and a subject amino acid sequence is the “Identities” value, expressed as a percentage, which is calculated by the BLASTP algorithm when a subject amino acid sequence has 100% query coverage with a query amino acid sequence after a pair-wise BLASTP alignment is performed. Such pair-wise BLASTP alignments between a query amino acid sequence and a subject amino acid sequence are performed by using the default settings of the BLASTP algorithm available on the National Center for Biotechnology Institute's website with the filter for low complexity regions turned off. Importantly, a query amino acid sequence may be described by an amino acid sequence identified in one or more claims herein.

In any embodiment of a combination of the invention, or a method or use thereof, herein, the ABP may have any one or all CDRs, VH, VL, HC, LC, with 99, 98, 97, 96, 95, 94, 93, 92, 91, or 90, or 85, or 80, or 75, or 70 percent identity to the sequence shown or referenced, e.g. as defined by a SEQ ID NO disclosed herein.

ABPs that bind human OX40 receptor are provided herein (i.e. an anti-OX40 ABP and an anti-human OX40 receptor (hOX-40R) antibody, sometimes referred to herein as an “anti-OX40 ABP” or an “anti-OX40 antibody” and/or other variations of the same). These antibodies are useful in the treatment or prevention of acute or chronic diseases or conditions whose pathology involves OX40 signalling. In one aspect, an antigen binding protein, or isolated human antibody or functional fragment of such protein or antibody, that binds to human OX40R and is effective as a cancer treatment or treatment against disease is described, for example in combination with another compound such as an anti-PD-1 ABP, suitably an antagonist anti-PD1 ABP. Any of the antigen binding proteins or antibodies disclosed herein may be used as a medicament. Any one or more of the antigen binding proteins or antibodies may be used in the methods or compositions to treat cancer, e.g. those disclosed herein.

The isolated antibodies as described herein bind to OX40, and may bind to OX40 encoded from the following genes: NCBI Accession Number NP_003317, Genpept Accession Number P23510, or genes having 90 percent homology or 90 percent identity thereto. The isolated antibody provided herein may further bind to the OX40 receptor having one of the following GenBank Accession Numbers: AAB39944, CAE11757, or AAI05071.

Antigen binding proteins and antibodies that bind and/or modulate OX-40 receptor are known in the art. Exemplary anti-OX40 ABPs of a combination of the invention, or a method or use thereof, are disclosed, for example in International Publication No. WO2013/028231 (PCT/US2012/024570), international filing date 9 Feb. 2012, and WO2012/027328 (PCT/US2011/048752), international filing date 23 Aug. 2011, each of which is incorporated by reference in its entirety herein (To the extent any definitions conflict, this instant application controls).

In one embodiment, the OX-40 antigen binding protein is one disclosed in WO2012/027328 (PCT/US2011/048752), international filing date 23 Aug. 2011. In another embodiment, the antigen binding protein comprises the CDRs of an antibody disclosed in WO2012/027328 (PCT/US2011/048752), international filing date 23 Aug. 2011, or CDRs with at least 90% (e.g., 90%, 91%, 92%, 93%, 94%, 95%, 96%, 97%, 98%, or 99%, 100%) sequence identity to the disclosed CDR sequences. In a further embodiment the antigen binding protein comprises a VH, a VL, or both of an antibody disclosed in WO2012/027328 (PCT/US2011/048752), international filing date 23 Aug. 2011, or a VH or a VL with at least 90% (e.g., 90%, 91%, 92%, 93%, 94%, 95%, 96%, 97%, 98%, or 99%, 100%) sequence identity to the disclosed VH or VL sequences.

In another embodiment, the OX-40 antigen binding protein is one disclosed in WO2013/028231 (PCT/US2012/024570), international filing date 9 Feb. 2012. In another embodiment, the antigen binding protein comprises the CDRs of an antibody disclosed in WO2013/028231 (PCT/US2012/024570), international filing date 9 Feb. 2012, or CDRs with at least 90% (e.g., 90%, 91%, 92%, 93%, 94%, 95%, 96%, 97%, 98%, or 99%, 100%) sequence identity to the disclosed CDR sequences. In a further embodiment the antigen binding protein comprises a VH, a VL, or both of an antibody disclosed in WO2013/028231 (PCT/US2012/024570), international filing date 9 Feb. 2012, or a VH or a VL with at least 90% (e.g., 90%, 91%, 92%, 93%, 94%, 95%, 96%, 97%, 98%, or 99%, 100%) sequence identity to the disclosed VH or VL sequences.

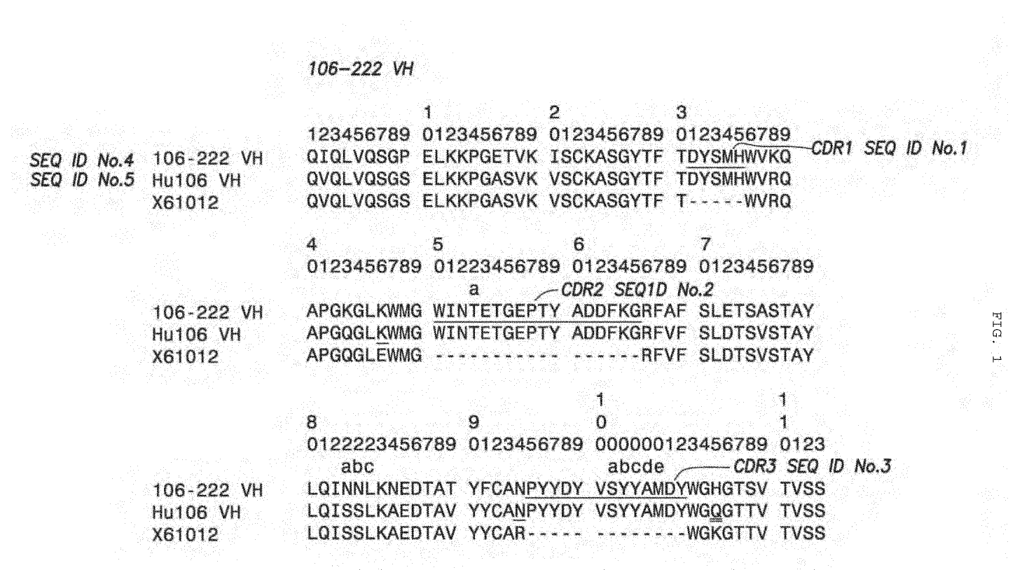

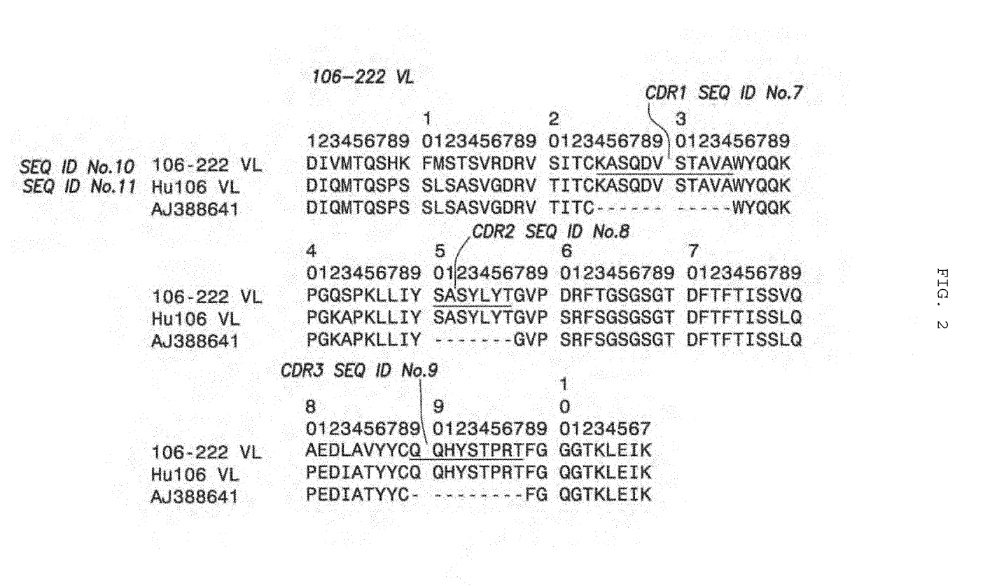

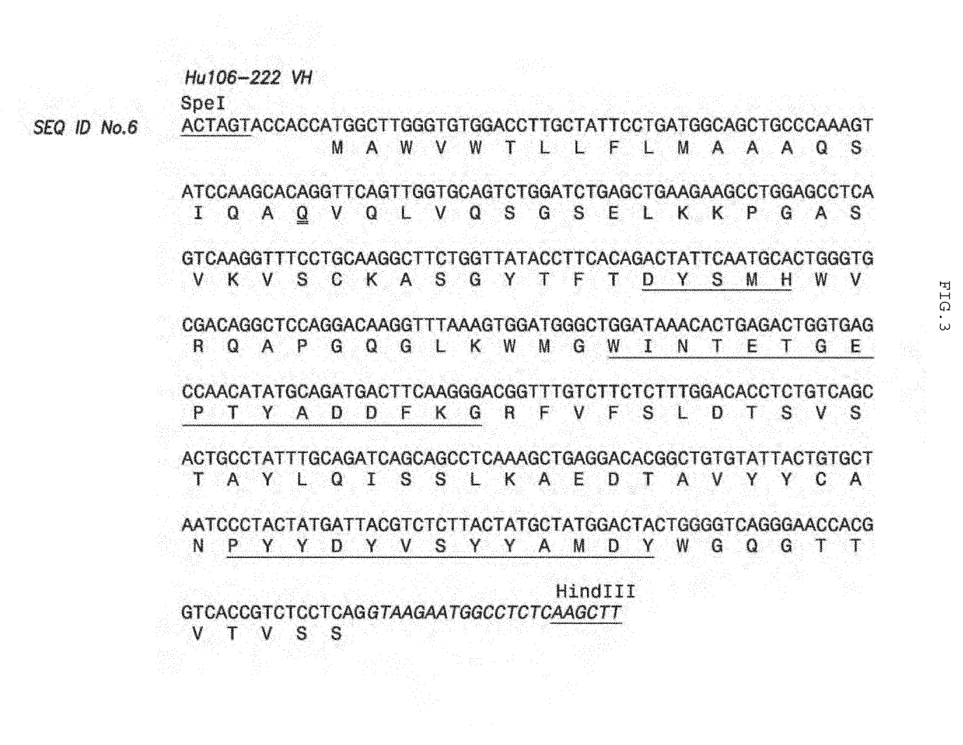

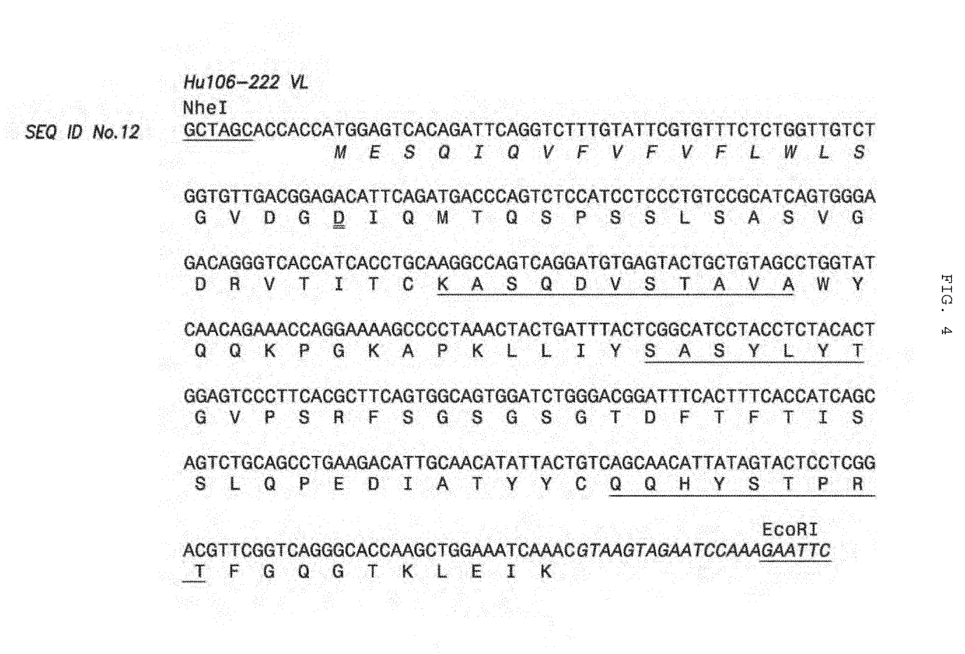

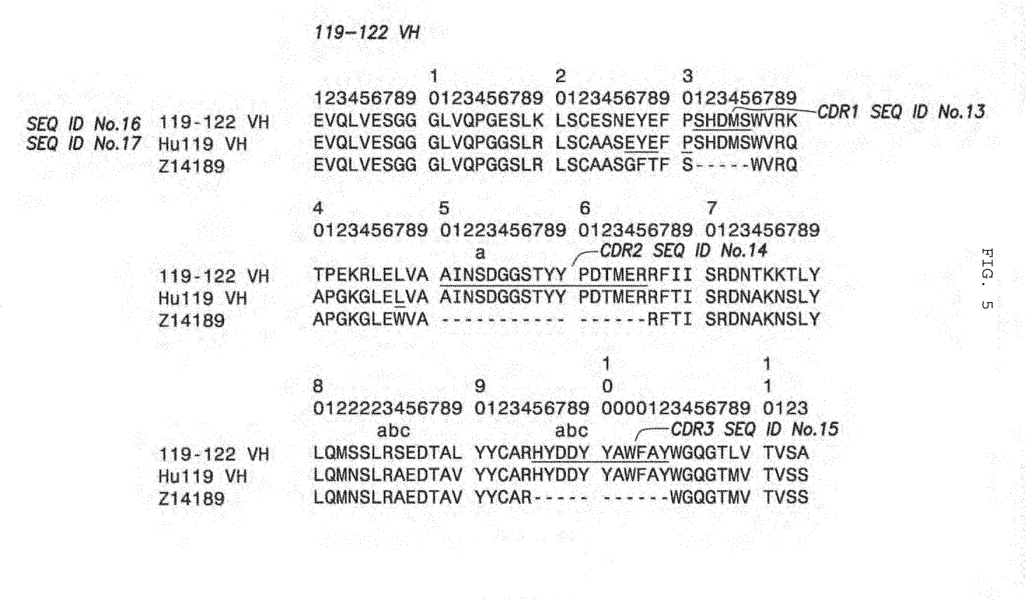

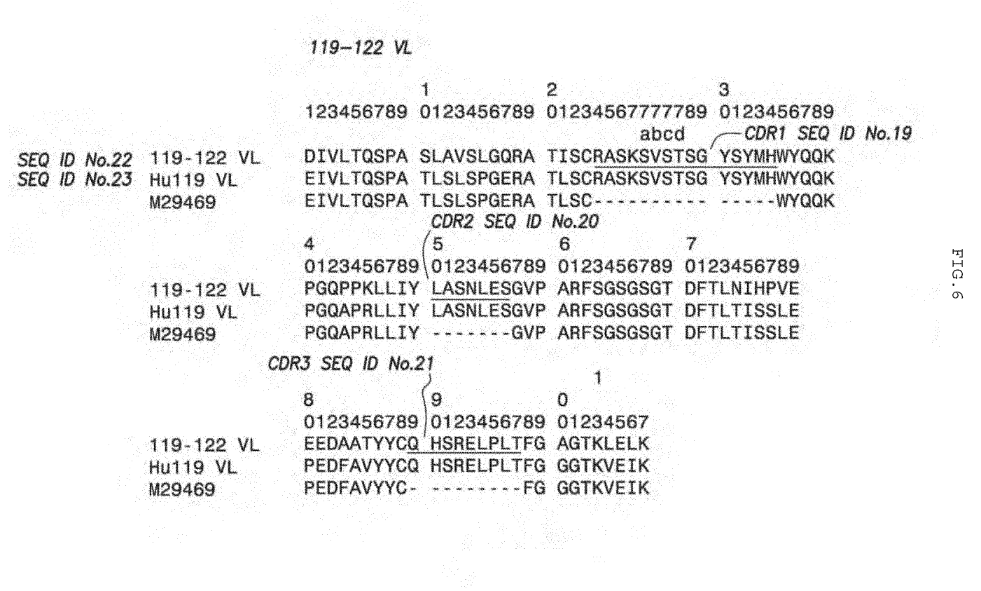

FIGS. 1-12 show sequences of the anti-OX40 ABPs of a combination of the invention, or a method or use thereof, e.g. CDRs and VH and VL sequences of the ABPs. In another embodiment, the anti-OX40 ABP of a combination of the invention, or a method or use thereof, comprises one or more of the CDRs or VH or VL sequences, or sequences with at least 90% (e.g., 90%, 91%, 92%, 93%, 94%, 95%, 96%, 97%, 98%, or 99%, 100%) sequence identity thereto, shown in the Figures herein. FIG. 1 includes a disclosure of residues 1-30, 36-49, 67-98, and 121-131 of SEQ ID NO: 108. X61012 is disclosed as SEQ ID NO: 108. FIG. 2 includes a disclosure of residues 1-23, 35-49, 57-88, and 102-111 of SEQ ID NO: 109. AJ388641 is disclosed as SEQ ID NO: 109. FIG. 3 includes a disclosure of the amino acid sequence as SEQ ID NO: 110. FIG. 4 includes a disclosure of the amino acid sequence as SEQ ID NO: 111. FIG. 5 includes a disclosure of residues 17-46, 52-65, 83-114, and 126-136 of SEQ ID NO: 112. Z14189 disclosed as SEQ ID NO: 112. FIG. 6 includes a disclosure of residues 21-43, 55-69, 77-108, and 118-127 of SEQ ID NO: 113. M29469 is disclosed as SEQ ID NO: 113. FIG. 7 includes a disclosure of the amino acid sequence as SEQ ID NO: 114. FIG. 8 includes a disclosure of the amino acid sequence as SEQ ID NO: 115.

FIG. 1 shows the alignment of the amino acid sequences of 106-222, humanized 106-222 (Hu106), and human acceptor X61012 (GenBank accession number) VH sequences are shown. Amino acid residues are shown in single letter code. Numbers above the sequences indicate the locations according to Kabat et al. (Sequences of Proteins of Immunological Interests, Fifth edition, NIH Publication No. 91-3242, U.S. Department of Health and Human Services, 1991). The same sequences as claimed herein are also provided in the Sequence Listing and the position numbers may be different. In FIG. 1, CDR sequences defined by Kabat et al. (1991) are underlined in 106-222 VH. CDR residues in X61012 VH are omitted in the figure. Human VH sequences homologous to the 106-222 VH frameworks were searched for within the GenBank database, and the VH sequence encoded by the human X61012 cDNA (X61012 VH) was chosen as an acceptor for humanization. The CDR sequences of 106-222 VH were first transferred to the corresponding positions of X61012 VH. Next, at framework positions where the three-dimensional model of the 106-222 variable regions suggested significant contact with the CDRs, amino acid residues of mouse 106-222 VH were substituted for the corresponding human residues. These substitutions were performed at positions 46 and 94 (underlined in Hu106 VH). In addition, a human framework residue that was found to be atypical in the corresponding V region subgroup was substituted with the most typical residue to reduce potential immunogenicity. This substitution was performed at position 105 (double-underlined in Hu106 VH).

FIG. 2 shows alignment of the amino acid sequences of 106-222, humanized 106-222 (Hu106), and human acceptor AJ388641 (GenBank accession number) VL sequences is shown. Amino acid residues are shown in single letter code. Numbers above the sequences indicate the locations according to Kabat et al. (1991). The same sequences as claimed herein are also provided in the Sequence Listing although the position numbers may be different. CDR sequences defined by Kabat et al. are underlined in 106-222 VH. CDR residues in AJ388641 VL are omitted in the figure. Human VL sequences homologous to the 106-222 VL frameworks were searched for within the GenBank database, and the VL sequence encoded by the human AJ388641 cDNA (AJ388641 VL) was chosen as an acceptor for humanization. The CDR sequences of 106-222 VL were transferred to the corresponding positions of AJ388641 VL. No framework substitutions were performed in the humanized form.

FIG. 3 shows the nucleotide sequence of the Hu106 VH gene flanked by SpeI and HindIII sites (underlined) is shown along with the deduced amino acid sequence. Amino acid residues are shown in single letter code. The signal peptide sequence is in italic. The N-terminal amino acid residue (Q) of the mature VH is double-underlined. CDR sequences according to the definition of Kabat et al. (1991) are underlined. The same sequences as claimed herein are also provided in the Sequence Listing and the position numbers may be different in the Sequence Listing. The intron sequence is in italic.

FIG. 4 shows the nucleotide sequence of the Hu106-222 VL gene flanked by NheI and EcoRI sites (underlined) is shown along with the deduced amino acid sequence. Amino acid residues are shown in single letter code. The signal peptide sequence is in italic. The N-terminal amino acid residue (D) of the mature VL is double-underlined. CDR sequences according to the definition of Kabat et al. (1991) are underlined. The intron sequence is in italic. The same sequences as claimed herein are also provided in the Sequence Listing although the position numbers may be different in the Sequence Listing.

FIG. 5 shows the alignment of the amino acid sequences of 119-122, humanized 119-122 (Hu119), and human acceptor Z14189 (GenBank accession number) VH sequences are shown. Amino acid residues are shown in single letter code. Numbers above the sequences indicate the locations according to Kabat et al. (Sequences of Proteins of Immunological Interests, Fifth edition, NIH Publication No. 91-3242, U.S. Department of Health and Human Services, 1991). CDR sequences defined by Kabat et al. (1991) are underlined in 119-122 VH. CDR residues in Z14189 VH are omitted in the figure. Human VH sequences homologous to the 119-122 VH frameworks were searched for within the GenBank database, and the VH sequence encoded by the human Z14189 cDNA (Z14189 VH) was chosen as an acceptor for humanization. The CDR sequences of 119-122 VH were first transferred to the corresponding positions of Z14189 VH. Next, at framework positions where the three-dimensional model of the 119-122 variable regions suggested significant contact with the CDRs, amino acid residues of mouse 119-122 VH were substituted for the corresponding human residues. These substitutions were performed at positions 26, 27, 28, 30 and 47 (underlined in the Hu119 VH sequence) as shown on the figure. The same sequences as claimed herein are also provided in the Sequence Listing although the position numbers may be different in the Sequence Listing.

FIG. 6 shows the alignment of the amino acid sequences of 119-122, humanized 119-122 (Hu119), and human acceptor M29469 (GenBank accession number) VL sequences are shown. Amino acid residues are shown in single letter code. Numbers above the sequences indicate the locations according to Kabat et al. (1991). CDR sequences defined by Kabat et al. (1) are underlined in 119-122 VL. CDR residues in M29469 VL are omitted in the sequence. Human VL sequences homologous to the 119-122 VL frameworks were searched for within the GenBank database, and the VL sequence encoded by the human M29469 cDNA (M29469 VL) was chosen as an acceptor for humanization. The CDR sequences of 119-122 VL were transferred to the corresponding positions of M29469 VL. No framework substitutions were needed in the humanized form. The same sequences as claimed herein are also provided in the Sequence Listing although the position numbers may be different in the Sequence Listing.

FIG. 7 shows the nucleotide sequence of the Hu119 VH gene flanked by SpeI and HindIII sites (underlined) is shown along with the deduced amino acid sequence. Amino acid residues are shown in single letter code. The signal peptide sequence is in italic. The N-terminal amino acid residue (E) of the mature VH is double-underlined. CDR sequences according to the definition of Kabat et al. (1991) are underlined. The intron sequence is in italic. The same sequences as claimed herein are also provided in the Sequence Listing although the position numbers may be different in the Sequence Listing.

FIG. 8 shows the nucleotide sequence of the Hu119 VL gene flanked by NheI and EcoRI sites (underlined) is shown along with the deduced amino acid sequence. Amino acid residues are shown in single letter code. The signal peptide sequence is in italic. The N-terminal amino acid residue (E) of the mature VL is double-underlined. CDR sequences according to the definition of Kabat et al. (1991) are underlined. The intron sequence is in italic. The same sequences as claimed herein are also provided in the Sequence Listing although the position numbers may be different in the Sequence Listing.

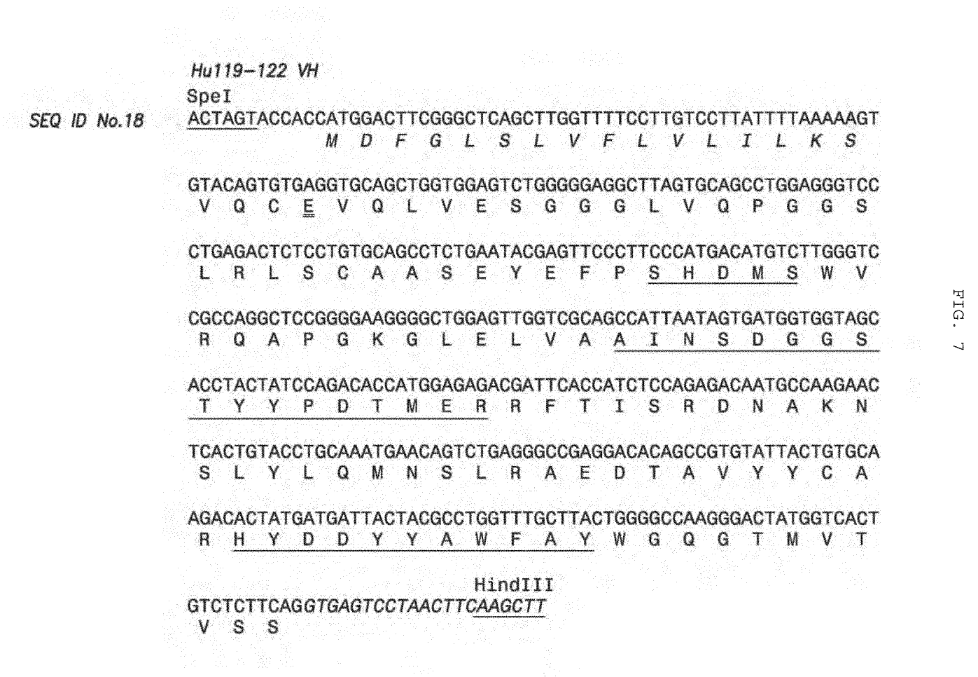

FIG. 9 shows the nucleotide sequence of mouse 119-43-1 VH cDNA along with the deduced amino acid sequence. Amino acid residues are shown in single letter code. The signal peptide sequence is in italic. The N-terminal amino acid residue (E) of the mature VH is double-underlined. CDR sequences according to the definition of Kabat et al. (Sequences of Proteins of Immunological Interests, Fifth edition, NIH Publication No. 91-3242, U.S. Department of Health and Human Services, 1991) are underlined.

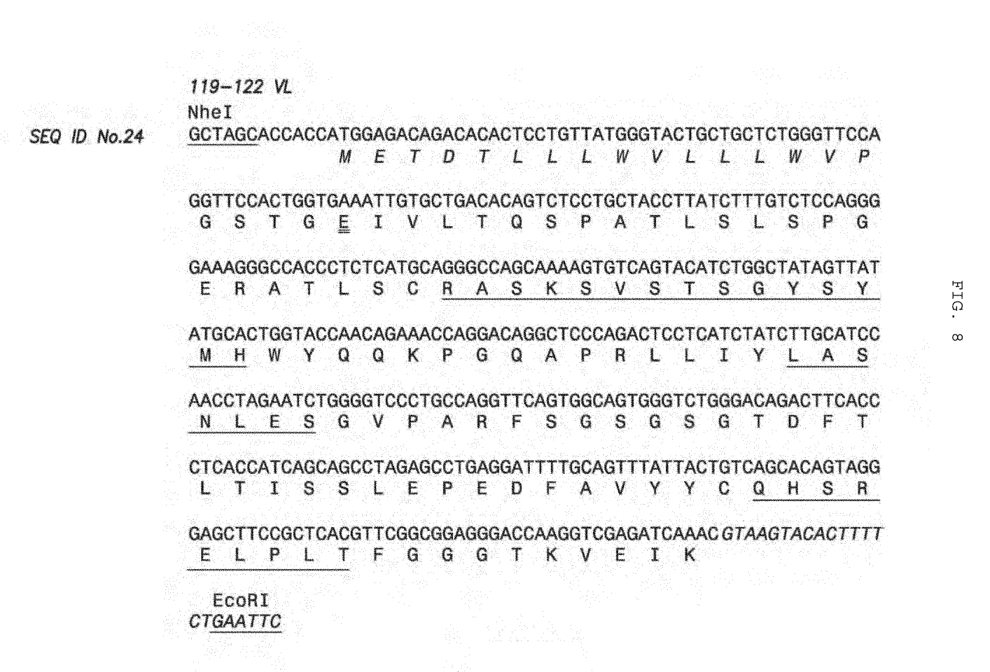

FIG. 10 shows the nucleotide sequence of mouse 119-43-1 VL cDNA is shown the deduced amino acid sequence. Amino acid residues are shown in single letter code. The signal peptide sequence is in italic. The N-terminal amino acid residue (D) of the mature VL is double-underlined. CDR sequences according to the definition of Kabat et al. (1991) are underlined.

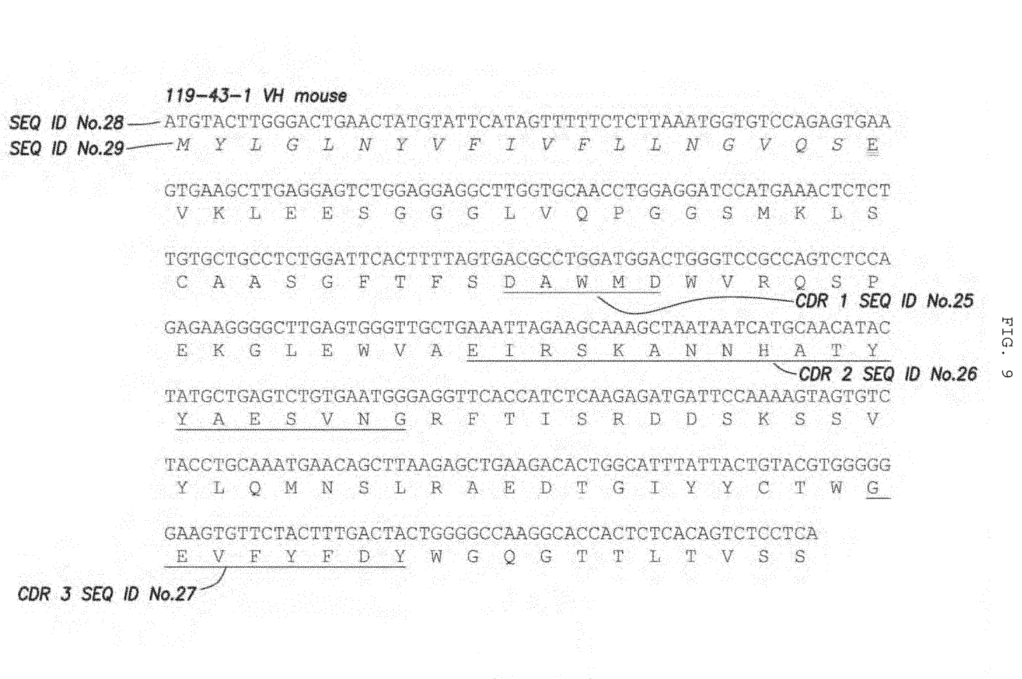

FIG. 11 shows the nucleotide sequence of the designed 119-43-1 VH gene flanked by SpeI and HindIII sites (underlined) along with the deduced amino acid sequence. Amino acid residues are shown in single letter code. The signal peptide sequence is in italic. The N-terminal amino acid residue (E) of the mature VH is double-underlined. CDR sequences according to the definition of Kabat et al. (1991) are underlined. The intron sequence is in italic.

FIG. 12 shows the nucleotide sequence of the designed 119-43-1 VL gene flanked by NheI and EcoRI sites (underlined) along with the deduced amino acid sequence. Amino acid residues are shown in single letter code. The signal peptide sequence is in italic. The N-terminal amino acid residue (D) of the mature VL is double-underlined. CDR sequences according to the definition of Kabat et al. (1991) are underlined. The intron sequence is in italic.

In one embodiment, the anti-OX40 ABP of a combination of the invention, or a method or use thereof, comprises the CDRs of the 106-222 antibody, e.g. CDRH1, CDRH2, and CDRH3 having the amino acid sequence as set forth in SEQ ID NOs 1, 2, and 3, and e.g. CDRL1, CDRL2, and CDRL3 having the sequences as set forth in SEQ ID NOs 7, 8, and 9 respectively. In one embodiment, the ABP of a combination of the invention, or a method or use thereof, comprises the CDRs of the 106-222, Hu106 or Hu106-222 antibody as disclosed in WO2012/027328 (PCT/US2011/048752), international filing date 23 Aug. 2011. In a further embodiment, the anti-OX40 ABP of a combination of the invention, or a method or use thereof, comprises the VH and VL regions of the 106-222 antibody as shown in FIG. 6 and FIG. 7 herein, e.g. a VH having an amino acid sequence as set forth in SEQ ID NO:4 and a VL having an amino acid sequence as set forth in SEQ ID NO: 10.

As described herein, ANTIBODY 106-222 is a humanized monoclonal antibody that binds to human OX40 as disclosed in WO2012/027328 and described herein an antibody comprising CDRH1, CDRH2, and CDRH3 having the amino acid sequence as set forth in SEQ ID NOs 1, 2, and 3, and e.g. CDRL1, CDRL2, and CDRL3 having the sequences as set forth in SEQ ID NOs 7, 8, and 9, respectively and an antibody comprising VH having an amino acid sequence as set forth in SEQ ID NO:4 and a VL having an amino acid sequence as set forth in SEQ ID NO: 10.

In another embodiment, the ABP of a combination of the invention, or a method or use thereof, comprises a VH having an amino acid sequence as set forth in SEQ ID NO: 5, and a VL having an amino acid sequence as set forth in SEQ ID NO:11. In a further embodiment, the anti-OX40 ABP of a combination of the invention, or a method or use thereof, comprises the VH and VL regions of the Hu106-222 antibody or the 106-222 antibody or the Hu106 antibody as disclosed in WO2012/027328 (PCT/US2011/048752), international filing date 23 Aug. 2011. In a further embodiment, the anti-OX40 ABP of a combination of the invention, or a method or use thereof, is 106-222, Hu106-222 or Hu106, e.g. as disclosed in WO2012/027328 (PCT/US2011/048752), international filing date 23 Aug. 2011. In a further embodiment, the ABP of a combination of the invention, or a method or use thereof, comprises CDRs or VH or VL or antibody sequences with at least 90% (e.g., 90%, 91%, 92%, 93%, 94%, 95%, 96%, 97%, 98%, or 99%, 100%) sequence identity to the sequences in this paragraph.

In another embodiment, the anti-OX40 ABP of a combination of the invention, or a method or use thereof, comprises the CDRs of the 119-122 antibody, e.g. CDRH1, CDRH2, and CDRH3 having the amino acid sequence as set forth in SEQ ID NOs 13, 14, and 15 respectively. In another embodiment, the anti-OX40 ABP of a combination of the invention, or a method or use thereof, comprises the CDRs of the 119-122 or Hu119 or Hu119-222 antibody as disclosed in WO2012/027328 (PCT/US2011/048752), international filing date 23 Aug. 2011. In a further embodiment, the anti-OX40 ABP of a combination of the invention, or a method or use thereof, comprises a VH having an amino acid sequence as set forth in SEQ ID NO: 16, and a VL having the amino acid sequence as set forth in SEQ ID NO: 22. In another embodiment, the anti-OX40 ABP of a combination of the invention, or a method or use thereof, comprises a VH having an amino acid sequence as set forth in SEQ ID NO: 17 and a VL having the amino acid sequence as set forth in SEQ ID NO: 23. In a further embodiment, the anti-OX40 ABP of a combination of the invention, or a method or use thereof, comprises the VH and VL regions of the 119-122 or Hu119 or Hu119-222 antibody as disclosed in WO2012/027328 (PCT/US2011/048752), international filing date 23 Aug. 2011. In a further embodiment, the ABP of a combination of the invention, or a method or use thereof, is 119-222 or Hu119 or Hu119-222 antibody, e.g. as disclosed in WO2012/027328 (PCT/US2011/048752), international filing date 23 Aug. 2011. In a further embodiment, the ABP comprises CDRs or VH or VL or antibody sequences with at least 90% (e.g., 90%, 91%, 92%, 93%, 94%, 95%, 96%, 97%, 98%, or 99%, 100%) sequence identity to the sequences in this paragraph.

In another embodiment, the anti-OX40 ABP of a combination of the invention, or a method or use thereof, comprises the CDRs of the 119-43-1 antibody as disclosed in WO2013/028231 (PCT/US2012/024570), international filing date 9 Feb. 2012. In another embodiment, the anti-OX40 ABP of a combination of the invention, or a method or use thereof, comprises the CDRs of the 119-43-1 antibody as disclosed in WO2013/028231 (PCT/US2012/024570), international filing date 9 Feb. 2012. In a further embodiment, the anti-OX40 ABP of a combination of the invention, or a method or use thereof, comprises one of the VH and one of the VL regions of the 119-43-1 antibody. In a further embodiment, the anti-OX40 ABP of a combination of the invention, or a method or use thereof, comprises the VH and VL regions of the 119-43-1 antibody as disclosed in WO2013/028231 (PCT/US2012/024570), international filing date 9 Feb. 2012. In a further embodiment, the anti-OX40 ABP of a combination of the invention, or a method or use thereof, is 119-43-1 or 119-43-1 chimeric. In a further embodiment, the anti-OX40 ABP of a combination of the invention, or a method or use thereof, as disclosed in WO2013/028231 (PCT/US2012/024570), international filing date 9 Feb. 2012. In further embodiments, any one of the anti-OX40 ABPs described in this paragraph are humanized. In further embodiments, any one of the any one of the ABPs described in this paragraph are engineered to make a humanized antibody. In a further embodiment, the anti-OX40 ABP of a combination of the invention, or a method or use thereof, comprises CDRs or VH or VL or antibody sequences with at least 90% (e.g., 90%, 91%, 92%, 93%, 94%, 95%, 96%, 97%, 98%, or 99%, 100%) sequence identity to the sequences in this paragraph.

In another embodiment, further embodiment, any mouse or chimeric sequences of any anti-OX40 ABP of a combination of the invention, or a method or use thereof, are engineered to make a humanized antibody.

In one embodiment, the anti-OX40 ABP of a combination of the invention, or a method or use thereof, comprises: (a) a heavy chain variable region CDR1 comprising the amino acid sequence of SEQ ID NO: 1; (b) a heavy chain variable region CDR2 comprising the amino acid sequence of SEQ ID NO: 2; (c) a heavy chain variable region CDR3 comprising the amino acid sequence of SEQ ID NO. 3; (d) a light chain variable region CDR1 comprising the amino acid sequence of SEQ ID NO. 7; (e) a light chain variable region CDR2 comprising the amino acid sequence of SEQ ID NO. 8; and (f) a light chain variable region CDR3 comprising the amino acid sequence of SEQ ID NO. 9.

In another embodiment, the anti-OX40 ABP of a combination of the invention, or a method or use thereof, comprises: (a) a heavy chain variable region CDR1 comprising the amino acid sequence of SEQ ID NO: 13; (b) a heavy chain variable region CDR2 comprising the amino acid sequence of SEQ ID NO: 14; (c) a heavy chain variable region CDR3 comprising the amino acid sequence of SEQ ID NO. 15; (d) a light chain variable region CDR1 comprising the amino acid sequence of SEQ ID NO. 19; (e) a light chain variable region CDR2 comprising the amino acid sequence of SEQ ID NO. 20; and (f) a light chain variable region CDR3 comprising the amino acid sequence of SEQ ID NO. 21.

In another embodiment, the anti-OX40 ABP of a combination of the invention, or a method or use thereof, comprises: a heavy chain variable region CDR1 comprising the amino acid sequence of SEQ ID NO: 1 or 13; a heavy chain variable region CDR2 comprising the amino acid sequence of SEQ ID NO: 2 or 14; and/or a heavy chain variable region CDR3 comprising the amino acid sequence of SEQ ID NO: 3 or 15, or a heavy chain variable region CDR having 90 percent identity thereto.

In another embodiment, the anti-OX40 ABP of a combination of the invention, or a method or use thereof, comprises: a light chain variable region CDR1 comprising the amino acid sequence of SEQ ID NO: 7 or 19; a light chain variable region CDR2 comprising the amino acid sequence of SEQ ID NO: 8 or 20 and/or a light chain variable region CDR3 comprising the amino acid sequence of SEQ ID NO: 9 or 21, or a heavy chain variable region having 90 percent identity thereto.

In another embodiment, the anti-OX40 ABP of a combination of the invention, or a method or use thereof, comprises: a light chain variable region (“VL”) comprising the amino acid sequence of SEQ ID NO: 10, 11, 22 or 23, or an amino acid sequence with at least 90% (e.g., 90, 91, 92, 93, 94, 95, 96, 97, 98, or 99%) identity to the amino acid sequences of SEQ ID NO: 10, 11, 22 or 23. In another embodiment, the anti-OX40 ABP of a combination of the invention, or a method or use thereof, comprises a heavy chain variable region (“VH”) comprising the amino acid sequence of SEQ ID NO: 4, 5, 16 and 17, or an amino acid sequence with at least 90% (e.g., 90, 91, 92, 93, 94, 95, 96, 97, 98, or 99%) identity to the amino acid sequences of SEQ ID NO: 4, 5, 16 and 17. In another embodiment, the anti-OX40 ABP of a combination of the invention, or a method or use thereof, comprises a variable heavy sequence of SEQ ID NO:5 and a variable light sequence of SEQ ID NO: 11, or a sequence having 90 percent identity thereto. In another embodiment, the anti-OX40 ABP of a combination of the invention, or a method or use thereof, comprises a variable heavy sequence of SEQ ID NO:17 and a variable light sequence of SEQ ID NO: 23 or a sequence having 90 percent identity thereto.

In another embodiment, the anti-OX40 ABP of a combination of the invention, or a method or use thereof, comprises a variable light chain encoded by the nucleic acid sequence of SEQ ID NO: 12, or 24, or a nucleic acid sequence with at least 90% (e.g., 90, 91, 92, 93, 94, 95, 96, 97, 98, or 99%) identity to the nucleotide sequences of SEQ ID NO: 12 or 24. In another embodiment, the anti-OX40 ABP of a combination of the invention, or a method or use thereof, comprises a variable heavy chain encoded by a nucleic acid sequence of SEQ ID NO: 6 or 18, or a nucleic acid sequence with at least 90% (e.g., 90, 91, 92, 93, 94, 95, 96, 97, 98, or 99%) identity to nucleotide sequences of SEQ ID NO: 6 or 18.

Also provided herein are monoclonal antibodies. In one embodiment, the monoclonal antibodies comprise a variable light chain comprising the amino acid sequence of SEQ ID NO: 10 or 22, or an amino acid sequence with at least 90% (e.g., 90, 91, 92, 93, 94, 95, 96, 97, 98, or 99%) identity to the amino acid sequences of SEQ ID NO: 10 or 22. Further provided are monoclonal antibodies comprising a variable heavy chain comprising the amino acid sequence of SEQ ID NO: 4 or 16, or an amino acid sequence with at least 90% (e.g., 90, 91, 92, 93, 94, 95, 96, 97, 98, or 99%) identity to the amino acid sequences of SEQ ID NO: 4 or 16.

Also provided herein are monoclonal antibodies. In one embodiment, the monoclonal antibodies comprise a variable light chain comprising the amino acid sequence of SEQ ID NO: 11 or 23, or an amino acid sequence with at least 90% (e.g., 90, 91, 92, 93, 94, 95, 96, 97, 98, or 99%) identity to the amino acid sequences of SEQ ID NO: 11 or 23. Further provided are monoclonal antibodies comprising a variable heavy chain comprising the amino acid sequence of SEQ ID NO: 5 or 17, or an amino acid sequence with at least 90% (e.g., 90, 91, 92, 93, 94, 95, 96, 97, 98, or 99%) identity to the amino acid sequences of SEQ ID NO: 5 or 17.

Another embodiment of a combination of the invention, or a method or use thereof, includes CDRs, VH regions, and VL regions, and antibodies and nucleic acids encoding the same as disclosed in the below Sequence Listing.

```
Heavy Chain of ANTIBODY 106-222:
(SEQ ID NO: 48)
QVQLVQSGSELKKPGASVKVSCKASGYTFTDYSMHWVRQAPGQGLKWMGWINTETGEPTYADDFKGR
FVFSLDTSVSTAYLQISSLKAEDTAVYYCANPYYDYVSYYAMDYWGQGTTVTVSSASTKGPSVFPLA
PSSKSTSGGTAALGCLVKDYFPEPVTVSWNSGALTSGVHTFPAVLQSSGLYSLSSVVTVPSSSLGTQ
TYICNVNHKPSNTKVDKKVEPKSCDKTHTCPPCPAPELLGGPSVFLFPPKPKDTLMISRTPEVTCVV
VDVSHEDPEVKFNWYVDGVEVHNAKTKPREEQYNSTYRVVSVLTVLHQDWLNGKEYKCKVSNKALPA
PIEKTISKAKGQPREPQVYTLPPSRDELTKNQVSLTCLVKGFYPSDIAVEWESNGQPENNYKTTPPV
LDSDGSFFLYSKLTVDKSRWQQGNVFSCSVMHEALHNHYTQKSLSLSPGK
Light Chain of ANTIBODY 106-222:
(SEQ ID NO: 49)
DIQMTQSPSSLSASVGDRVTITCKASQDVSTAVAWYQQKPGKAPKLLIYSASYLYTGVPSRFSGSGS
GTDFTFTISSLQPEDIATYYCQQHYSTPRTFGQGTKLEIKRTVAAPSVFIFPPSDEQLKSGTASVVC
LLNNFYPREAKVQWKVDNALQSGNSQESVTEQDSKDSTYSLSSTLTLSKADYEKHKVYACEVTHQGL
SSPVTKSFNRGEC
Heavy Chain Variable Region of ANTIBODY 106-222:
(SEQ ID NO: 5)
QVQLVQSGSELKKPGASVKVSCKASGYTFTDYSMHWVRQAPGQGLKWMGWINTETGEPTYADDFKGR
FVFSLDTSVSTAYLQISSLKAEDTAVYYCANPYYDYVSYYAMDYWGQGTTVTVSS
Light Chain Variable Region of ANTIBODY 106-222:
(SEQ ID NO: 11)
DIQMTQSPSSLSASVGDRVTITCKASQDVSTAVAWYQQKPGKAPKLLIYSASYLYTGVPSRFSGSGS
GTDFTFTISSLQPEDIATYYCQQHYSTPRTFGQGTKLEIK
CDR sequences of ANTIBODY 106-222:
HC CDR1:
(SEQ ID NO: 1)
Asp Tyr Ser Met His
HC CDR2:
(SEQ ID NO: 2)
Trp Ile Asn Thr Glu Thr Gly Glu Pro Thr Tyr Ala Asp Asp Phe Lys Gly
HC CDR3:
(SEQ ID NO: 3)
Pro Tyr Tyr Asp Tyr Val Ser Tyr Tyr Ala Met Asp Tyr
LC CDR1:
(SEQ ID NO: 7)
Lys Ala Ser Gln Asp Val Ser Thr Ala Val Ala
LC CDR2:
(SEQ ID NO: 8)
Ser Ala Ser Tyr Leu Tyr Thr
LC CDR3:
(SEQ ID NO: 9)
Gln Gln His Tyr Ser Thr Pro Arg Thr
OX40 Antibody Sequence Listing
<140> UNKNOWN
<141> 2014-02-24
<150> PCT/US2012/024570
<151> 2012-02-09
<150> PCT/US2011/048752
<151> 2011-08-23
<160> 47
<170> PatentIn version 3.5
<210> 1
<211> 5
<212> PRT
<213> Mus sp.
<400> 1
Asp Tyr Ser Met His
1 5
<210> 2
<211> 17
<212> PRT
<213> Mus sp.
<400> 2
Trp Ile Asn Thr Glu Thr Gly Glu Pro Thr Tyr Ala Asp Asp Phe Lys
1 5 10 15
Gly
<210> 3
<211> 13
<212> PRT
<213> Mus sp.
<400> 3
Pro Tyr Tyr Asp Tyr Val Ser Tyr Tyr Ala Met Asp Tyr
1 5 10
<210> 4
<211> 122
<212> PRT
<213> Mus sp.
<400> 4
Gln Ile Gln Leu Val Gln Ser Gly Pro Glu Leu Lys Lys Pro Gly Glu
1 5 10 15
Thr Val Lys Ile Ser Cys Lys Ala Ser Gly Tyr Thr Phe Thr Asp Tyr
                        20 25 30
Ser Met His Trp Val Lys Gln Ala Pro Gly Lys Gly Leu Lys Trp Met
                35 40 45
Gly Trp Ile Asn Thr Glu Thr Gly Glu Pro Thr Tyr Ala Asp Asp Phe
        50 55 60
Lys Gly Arg Phe Ala Phe Ser Leu Glu Thr Ser Ala Ser Thr Ala Tyr
65 70 75 80
Leu Gln Ile Asn Asn Leu Lys Asn Glu Asp Thr Ala Thr Tyr Phe Cys
                                85 90 95
Ala Asn Pro Tyr Tyr Asp Tyr Val Ser Tyr Tyr Ala Met Asp Tyr Trp
                        100 105 110
Gly Gln Gly Thr Thr Val Thr Val Ser Ser
                115 120
<210> 5
<211> 122
<212> PRT
<213> Artificial Sequence
<220>
<223> Description of Artificial Sequence: Synthetic polypeptide
<400> 5
Gln Val Gln Leu Val Gln Ser Gly Ser Glu Leu Lys Lys Pro Gly Ala
1 5 10 15
Ser Val Lys Val Ser Cys Lys Ala Ser Gly Tyr Thr Phe Thr Asp Tyr
                        20 25 30
Ser Met His Trp Val Arg Gln Ala Pro Gly Gln Gly Leu Lys Trp Met
                35 40 45
Gly Trp Ile Asn Thr Glu Thr Gly Glu Pro Thr Tyr Ala Asp Asp Phe
        50 55 60
Lys Gly Arg Phe Val Phe Ser Leu Asp Thr Ser Val Ser Thr Ala Tyr
65 70 75 80
Leu Gln Ile Ser Ser Leu Lys Ala Glu Asp Thr Ala Val Tyr Tyr Cys
                                85 90 95
Ala Asn Pro Tyr Tyr Asp Tyr Val Ser Tyr Tyr Ala Met Asp Tyr Trp
                        100 105 110
Gly Gln Gly Thr Thr Val Thr Val Ser Ser
                115 120
<210> 6
<211> 458
<212> DNA
<213> Artificial Sequence
<220>
<223> Description of Artificial Sequence: Synthetic polynucleotide
<400> 6
actagtacca ccatggcttg ggtgtggacc ttgctattcc tgatggcagc tgcccaaagt
60
atccaagcac aggttcagtt ggtgcagtct ggatctgagc tgaagaagcc tggagcctca
120
gtcaaggttt cctgcaaggc ttctggttat accttcacag actattcaat gcactgggtg
180
cgacaggctc caggacaagg tttaaagtgg atgggctgga taaacactga gactggtgag
240
ccaacatatg cagatgactt caagggacgg tttgtcttct ctttggacac ctctgtcagc
300
actgcctatt tgcagatcag cagcctcaaa gctgaggaca cggctgtgta ttactgtgct
360
aatccctact atgattacgt ctcttactat gctatggact actggggtca gggaaccacg
420
gtcaccgtct cctcaggtaa gaatggcctc tcaagctt
458
<210> 7
<211> 11
<212> PRT
<213> Mus sp.
<400> 7
Lys Ala Ser Gln Asp Val Ser Thr Ala Val Ala
1 5 10
<210> 8
<211> 7
<212> PRT
<213> Mus sp.
<400> 8
Ser Ala Ser Tyr Leu Tyr Thr
1 5
<210> 9
<211> 9
<212> PRT
<213> Mus sp.
<400> 9
Gln Gln His Tyr Ser Thr Pro Arg Thr
1 5
<210> 10
<211> 107
<212> PRT
<213> Mus sp.
<400> 10
Asp Ile Val Met Thr Gln Ser His Lys Phe Met Ser Thr Ser Val Arg
1 5 10 15
Asp Arg Val Ser Ile Thr Cys Lys Ala Ser Gln Asp Val Ser Thr Ala
                        20 25 30
Val Ala Trp Tyr Gln Gln Lys Pro Gly Gln Ser Pro Lys Leu Leu Ile
                35 40 45
Tyr Ser Ala Ser Tyr Leu Tyr Thr Gly Val Pro Asp Arg Phe Thr Gly
        50 55 60
Ser Gly Ser Gly Thr Asp Phe Thr Phe Thr Ile Ser Ser Val Gln Ala
65 70 75 80
Glu Asp Leu Ala Val Tyr Tyr Cys Gln Gln His Tyr Ser Thr Pro Arg
                                85 90 95
Thr Phe Gly Gly Gly Thr Lys Leu Glu Ile Lys
                        100 105
<210> 11
<211> 107
<212> PRT
<213> Artificial Sequence
<220>
<223> Description of Artificial Sequence: Synthetic polypeptide
<400> 11
Asp Ile Gln Met Thr Gln Ser Pro Ser Ser Leu Ser Ala Ser Val Gly
1 5 10 15
Asp Arg Val Thr Ile Thr Cys Lys Ala Ser Gln Asp Val Ser Thr Ala
                        20 25 30
Val Ala Trp Tyr Gln Gln Lys Pro Gly Lys Ala Pro Lys Leu Leu Ile
                35 40 45
Tyr Ser Ala Ser Tyr Leu Tyr Thr Gly Val Pro Ser Arg Phe Ser Gly
        50 55 60
Ser Gly Ser Gly Thr Asp Phe Thr Phe Thr Ile Ser Ser Leu Gln Pro
65 70 75 80
Glu Asp Ile Ala Thr Tyr Tyr Cys Gln Gln His Tyr Ser Thr Pro Arg
                                85 90 95
Thr Phe Gly Gln Gly Thr Lys Leu Glu Ile Lys
                        100 105
<210> 12
<211> 416
<212> DNA
<213> Artificial Sequence
<220>
<223> Description of Artificial Sequence: Synthetic polynucleotide
<400> 12
gctagcacca ccatggagtc acagattcag gtctttgtat tcgtgtttct ctggttgtct
60
ggtgttgacg gagacattca gatgacccag tctccatcct ccctgtccgc atcagtggga
120
gacagggtca ccatcacctg caaggccagt caggatgtga gtactgctgt agcctggtat
180
caacagaaac caggaaaagc ccctaaacta ctgatttact cggcatccta cctctacact
240
ggagtccctt cacgcttcag tggcagtgga tctgggacgg atttcacttt caccatcagc
300
agtctgcagc ctgaagacat tgcaacatat tactgtcagc aacattatag tactcctcgg
360
acgttcggtc agggcaccaa gctggaaatc aaacgtaagt agaatccaaa gaattc
416
<210> 13
<211> 5
<212> PRT
<213> Mus sp.
<400> 13
Ser His Asp Met Ser
1 5
<210> 14
<211> 17
<212> PRT
<213> Mus sp.
<400> 14
Ala Ile Asn Ser Asp Gly Gly Ser Thr Tyr Tyr Pro Asp Thr Met Glu
1 5 10 15
Arg
<210> 15
<211> 11
<212> PRT
<213> Mus sp.
<400> 15
His Tyr Asp Asp Tyr Tyr Ala Trp Phe Ala Tyr
1 5 10
<210> 16
<211> 120
<212> PRT
<213> Mus sp.
<400> 16
Glu Val Gln Leu Val Glu Ser Gly Gly Gly Leu Val Gln Pro Gly Glu
1 5 10
Ser Leu Lys Leu Ser Cys Glu Ser Asn Glu Tyr Glu Phe Pro Ser His
                        20 25 30
Asp Met Ser Trp Val Arg Lys Thr Pro Glu Lys Arg Leu Glu Leu Val
                35 40 45
Ala Ala Ile Asn Ser Asp Gly Gly Ser Thr Tyr Tyr Pro Asp Thr Met
        50 55 60
Glu Arg Arg Phe Ile Ile Ser Arg Asp Asn Thr Lys Lys Thr Leu Tyr
65 70 75 80
Leu Gln Met Ser Ser Leu Arg Ser Glu Asp Thr Ala Leu Tyr Tyr Cys
                                85 90 95
Ala Arg His Tyr Asp Asp Tyr Tyr Ala Trp Phe Ala Tyr Trp Gly Gln
                        100 105 110
Gly Thr Leu Val Thr Val Ser Ala
                115 120
<210> 17
<211> 120
<212> PRT
<213> Artificial Sequence
<220>
<223> Description of Artificial Sequence: Synthetic polypeptide
<400> 17
Glu Val Gln Leu Val Glu Ser Gly Gly Gly Leu Val Gln Pro Gly Gly
1 5 10 15
Ser Leu Arg Leu Ser Cys Ala Ala Ser Glu Tyr Glu Phe Pro Ser His
                        20 25 30
Asp Met Ser Trp Val Arg Gln Ala Pro Gly Lys Gly Leu Glu Leu Val
                35 40 45
Ala Ala Ile Asn Ser Asp Gly Gly Ser Thr Tyr Tyr Pro Asp Thr Met
        50 55 60
Glu Arg Arg Phe Thr Ile Ser Arg Asp Asn Ala Lys Asn Ser Leu Tyr
65 70 75 80
Leu Gln Met Asn Ser Leu Arg Ala Glu Asp Thr Ala Val Tyr Tyr Cys
                                85 90 95
Ala Arg His Tyr Asp Asp Tyr Tyr Ala Trp Phe Ala Tyr Trp Gly Gln
                        100 105 110
Gly Thr Met Val Thr Val Ser Ser
                115 120
<210> 18
<211> 451
<212> DNA
<213> Artificial Sequence
<220>
<223> Description of Artificial Sequence: Synthetic polynucleotide
<400> 18
actagtacca ccatggactt cgggctcagc ttggttttcc ttgtccttat tttaaaaagt
60
gtacagtgtg aggtgcagct ggtggagtct gggggaggct tagtgcagcc tggagggtcc
120
ctgagactct cctgtgcagc ctctgaatac gagttccctt cccatgacat gtcttgggtc
180
cgccaggctc cggggaaggg gctggagttg gtcgcagcca ttaatagtga tggtggtagc
240
acctactatc cagacaccat ggagagacga ttcaccatct ccagagacaa tgccaagaac
300
tcactgtacc tgcaaatgaa cagtctgagg gccgaggaca cagccgtgta ttactgtgca
360
agacactatg atgattacta cgcctggttt gcttactggg gccaagggac tatggtcact
420
gtctcttcag gtgagtccta acttcaagct t
451
<210> 19
<211> 15
<212> PRT
<213> Mus sp.
<400> 19
Arg Ala Ser Lys Ser Val Ser Thr Ser Gly Tyr Ser Tyr Met His
1 5 10 15
<210> 20
<211> 7
<212> PRT
<213> Mus sp.
<400> 20
Leu Ala Ser Asn Leu Glu Ser
1 5
<210> 21
<211> 9
<212> PRT
<213> Mus sp.
<400> 21
Gln His Ser Arg Glu Leu Pro Leu Thr
1 5
<210> 22
<211> 111
<212> PRT
<213> Mus sp.
<400> 22
Asp Ile Val Leu Thr Gln Ser Pro Ala Ser Leu Ala Val Ser Leu Gly
1 5 10 15
Gln Arg Ala Thr Ile Ser Cys Arg Ala Ser Lys Ser Val Ser Thr Ser
                        20 25 30
Gly Tyr Ser Tyr Met His Trp Tyr Gln Gln Lys Pro Gly Gln Pro Pro
                35 40 45
Lys Leu Leu Ile Tyr Leu Ala Ser Asn Leu Glu Ser Gly Val Pro Ala
        50 55 60
Arg Phe Ser Gly Ser Gly Ser Gly Thr Asp Phe Thr Leu Asn Ile His
65 70 75 80
Pro Val Glu Glu Glu Asp Ala Ala Thr Tyr Tyr Cys Gln His Ser Arg
                                85 90 95
Glu Leu Pro Leu Thr Phe Gly Ala Gly Thr Lys Leu Glu Leu Lys
                        100 105 110
<210> 23
<211> 111
<212> PRT
<213> Artificial Sequence
<220>
<223> Description of Artificial Sequence: Synthetic polypeptide
<400> 23
Glu Ile Val Leu Thr Gln Ser Pro Ala Thr Leu Ser Leu Ser Pro Gly
1 5 10 15
Glu Arg Ala Thr Leu Ser Cys Arg Ala Ser Lys Ser Val Ser Thr Ser
                        20 25 30
Gly Tyr Ser Tyr Met His Trp Tyr Gln Gln Lys Pro Gly Gln Ala Pro
                35 40 45
Arg Leu Leu Ile Tyr Leu Ala Ser Asn Leu Glu Ser Gly Val Pro Ala
        50 55 60
Arg Phe Ser Gly Ser Gly Ser Gly Thr Asp Phe Thr Leu Thr Ile Ser
65 70 75 80
Ser Leu Glu Pro Glu Asp Phe Ala Val Tyr Tyr Cys Gln His Ser Arg
                                85 90 95
Glu Leu Pro Leu Thr Phe Gly Gly Gly Thr Lys Val Glu Ile Lys
                        100 105 110
<210> 24
<211> 428
<212> DNA
<213> Artificial Sequence
<220>
<223> Description of Artificial Sequence: Synthetic polynucleotide
<400> 24
gctagcacca ccatggagac agacacactc ctgttatggg tactgctgct ctgggttcca
60
ggttccactg gtgaaattgt gctgacacag tctcctgcta ccttatcttt gtctccaggg
120
gaaagggcca ccctctcatg cagggccagc aaaagtgtca gtacatctgg ctatagttat
180
atgcactggt accaacagaa accaggacag gctcccagac tcctcatcta tcttgcatcc
240
aacctagaat ctggggtccc tgccaggttc agtggcagtg ggtctgggac agacttcacc
300
ctcaccatca gcagcctaga gcctgaggat tttgcagttt attactgtca gcacagtagg
360
gagcttccgc tcacgttcgg cggagggacc aaggtcgaga tcaaacgtaa gtacactttt
420
ctgaattc
428
<210> 25
<211> 5
<212> PRT
<213> Mus sp.
<400> 25
Asp Ala Trp Met Asp
1 5
<210> 26
<211> 19
<212> PRT
<213> Mus sp.
<400> 26
Glu Ile Arg Ser Lys Ala Asn Asn His Ala Thr Tyr Tyr Ala Glu Ser
1 5 10 15
Val Asn Gly
<210> 27
<211> 8
<212> PRT
<213> Mus sp.
<400> 27
Gly Glu Val Phe Tyr Phe Asp Tyr
1 5
<210> 28
<211> 414
<212> DNA
<213> Mus sp.
<400> 28
atgtacttgg gactgaacta tgtattcata gtttttctct taaatggtgt ccagagtgaa
60
gtgaagcttg aggagtctgg aggaggcttg gtgcaacctg gaggatccat gaaactctct
120
tgtgctgcct ctggattcac ttttagtgac gcctggatgg actgggtccg ccagtctcca
180
gagaaggggc ttgagtgggt tgctgaaatt agaagcaaag ctaataatca tgcaacatac
240
tatgctgagt ctgtgaatgg gaggttcacc atctcaagag atgattccaa aagtagtgtc
300
tacctgcaaa tgaacagctt aagagctgaa gacactggca tttattactg tacgtggggg
360
gaagtgttct actttgacta ctggggccaa ggcaccactc tcacagtctc ctca
414
<210> 29
<211> 138
<212> PRT
<213> Mus sp.
<400> 29
Met Tyr Leu Gly Leu Asn Tyr Val Phe Ile Val Phe Leu Leu Asn Gly
1 5 10 15
Val Gln Ser Glu Val Lys Leu Glu Glu Ser Gly Gly Gly Leu Val Gln
                        20 25 30
Pro Gly Gly Ser Met Lys Leu Ser Cys Ala Ala Ser Gly Phe Thr Phe
                35 40 45
Ser Asp Ala Trp Met Asp Trp Val Arg Gln Ser Pro Glu Lys Gly Leu
        50 55 60
Glu Trp Val Ala Glu Ile Arg Ser Lys Ala Asn Asn His Ala Thr Tyr
65 70 75 80
Tyr Ala Glu Ser Val Asn Gly Arg Phe Thr Ile Ser Arg Asp Asp Ser
                                85 90 95
Lys Ser Ser Val Tyr Leu Gln Met Asn Ser Leu Arg Ala Glu Asp Thr
                        100 105 110
Gly Ile Tyr Tyr Cys Thr Trp Gly Glu Val Phe Tyr Phe Asp Tyr Trp
                115 120 125
Gly Gln Gly Thr Thr Leu Thr Val Ser Ser
        130 135
<210> 30
<211> 448
<212> DNA
<213> Artificial Sequence
<220>
<223> Description of Artificial Sequence: Synthetic polynucleotide
<400> 30
actagtacca ccatgtactt gggactgaac tatgtattca tagtttttct cttaaatggt
60
gtccagagtg aagtgaagct ggaggagtct ggaggaggct tggtgcaacc tggaggatcc
120
atgaaactct cttgtgctgc ctctggattc acttttagtg acgcctggat ggactgggtc
180
cgccagtctc cagagaaggg gcttgagtgg gttgctgaaa ttagaagcaa agctaataat
240
catgcaacat actatgctga gtctgtgaat gggaggttca ccatctcaag agatgattcc
300
aaaagtagtg tctacctgca aatgaacagc ttaagagctg aagacactgg catttattac
360
tgtacgtggg gggaagtgtt ctactttgac tactggggcc aaggcaccac tctcacagtc
420
tcctcaggtg agtccttaaa acaagctt
448
<210> 31
<211> 138
<212> PRT
<213> Artificial Sequence
<220>
<223> Description of Artificial Sequence: Synthetic polypeptide
<400> 31
Met Tyr Leu Gly Leu Asn Tyr Val Phe Ile Val Phe Leu Leu Asn Gly
1 5 10 15
Val Gln Ser Glu Val Lys Leu Glu Glu Ser Gly Gly Gly Leu Val Gln
                        20 25 30
Pro Gly Gly Ser Met Lys Leu Ser Cys Ala Ala Ser Gly Phe Thr Phe
                35 40 45
Ser Asp Ala Trp Met Asp Trp Val Arg Gln Ser Pro Glu Lys Gly Leu
        50 55 60
Glu Trp Val Ala Glu Ile Arg Ser Lys Ala Asn Asn His Ala Thr Tyr
65 70 75 80
Tyr Ala Glu Ser Val Asn Gly Arg Phe Thr Ile Ser Arg Asp Asp Ser
                                85 90 95
Lys Ser Ser Val Tyr Leu Gln Met Asn Ser Leu Arg Ala Glu Asp Thr
                        100 105 110
Gly Ile Tyr Tyr Cys Thr Trp Gly Glu Val Phe Tyr Phe Asp Tyr Trp
                115 120 125
Gly Gln Gly Thr Thr Leu Thr Val Ser Ser
        130 135
<210> 32
<211> 11
<212> PRT
<213> Mus sp.
<400> 32
Lys Ser Ser Gln Asp Ile Asn Lys Tyr Ile Ala
1 5 10
<210> 33
<211> 7
<212> PRT
<213> Mus sp.
<400> 33
Tyr Thr Ser Thr Leu Gln Pro
1 5
<210> 34
<211> 8
<212> PRT
<213> Mus sp.
<400> 34
Leu Gln Tyr Asp Asn Leu Leu Thr
1 5
<210> 35
<211> 378
<212> DNA
<213> Mus sp.
<400> 35
atgagaccgt ctattcagtt cctggggctc ttgttgttct ggcttcatgg tgctcagtgt
60
gacatccaga tgacacagtc tccatcctca ctgtctgcat ctctgggagg caaagtcacc
120
atcacttgca agtcaagcca agacattaac aagtatatag cttggtacca acacaagcct
180
ggaaaaggtc ctaggctgct catacattac acatctacat tacagccagg catcccatca
240
aggttcagtg gaagtgggtc tgggagagat tattccttca gcatcagcaa cctggagcct
300
gaagatattg caacttatta ttgtctacag tatgataatc ttctcacgtt cggtgctggg
360
accaagctgg agctgaaa
378
<210> 36
<211> 126
<212> PRT
<213> Mus sp.
<400> 36
Met Arg Pro Ser Ile Gln Phe Leu Gly Leu Leu Leu Phe Trp Leu His
1 5 10 15
Gly Ala Gln Cys Asp Ile Gln Met Thr Gln Ser Pro Ser Ser Leu Ser
                        20 25 30
Ala Ser Leu Gly Gly Lys Val Thr Ile Thr Cys Lys Ser Ser Gln Asp
                35 40 45
Ile Asn Lys Tyr Ile Ala Trp Tyr Gln His Lys Pro Gly Lys Gly Pro
        50 55 60
Arg Leu Leu Ile His Tyr Thr Ser Thr Leu Gln Pro Gly Ile Pro Ser
65 70 75 80
Arg Phe Ser Gly Ser Gly Ser Gly Arg Asp Tyr Ser Phe Ser Ile Ser
                                85 90 95
Asn Leu Glu Pro Glu Asp Ile Ala Thr Tyr Tyr Cys Leu Gln Tyr Asp
                        100 105 110
Asn Leu Leu Thr Phe Gly Ala Gly Thr Lys Leu Glu Leu Lys
                115 120 125
<210> 37
<211> 413
<212> DNA
<213> Artificial Sequence
<220>
<223> Description of Artificial Sequence: Synthetic polynucleotide
<400> 37
gctagcacca ccatgagacc gtctattcag ttcctggggc tcttgttgtt ctggcttcat
60
ggtgctcagt gtgacatcca gatgacacag tctccatcct cactgtctgc atctctggga
120
ggcaaagtca ccatcacttg caagtcaagc caagacatta acaagtatat agcttggtac
180
caacacaagc ctggaaaagg tcctaggctg ctcatacatt acacatctac attacagcca
240
ggcatcccat caaggttcag tggaagtggg tctgggagag attattcctt cagcatcagc
300
aacctggagc ctgaagatat tgcaacttat tattgtctac agtatgataa tcttctcacg
360
ttcggtgctg ggaccaagct ggagctgaaa cgtaagtaca cttttctgaa ttc
413
<210> 38
<211> 126
<212> PRT
<213> Artificial Sequence
<220>
<223> Description of Artificial Sequence: Synthetic polypeptide
<400> 38
Met Arg Pro Ser Ile Gln Phe Leu Gly Leu Leu Leu Phe Trp Leu His
1 5 10 15
Gly Ala Gln Cys Asp Ile Gln Met Thr Gln Ser Pro Ser Ser Leu Ser
                        20 25 30
Ala Ser Leu Gly Gly Lys Val Thr Ile Thr Cys Lys Ser Ser Gln Asp
                35 40 45
Ile Asn Lys Tyr Ile Ala Trp Tyr Gln His Lys Pro Gly Lys Gly Pro
        50 55 60
Arg Leu Leu Ile His Tyr Thr Ser Thr Leu Gln Pro Gly Ile Pro Ser
65 70 75 80
Arg Phe Ser Gly Ser Gly Ser Gly Arg Asp Tyr Ser Phe Ser Ile Ser
                                85 90 95
Asn Leu Glu Pro Glu Asp Ile Ala Thr Tyr Tyr Cys Leu Gln Tyr Asp
                        100 105 110
Asn Leu Leu Thr Phe Gly Ala Gly Thr Lys Leu Glu Leu Lys
                115 120 125
<210> 39
<211> 20
<212> DNA
<213> Artificial Sequence
<220>
<223> Description of Artificial Sequence: Synthetic primer
<400> 39
cgctgttttg acctccatag
20
<210> 40
<211> 20
<212> DNA
<213> Artificial Sequence
<220>
<223> Description of Artificial Sequence: Synthetic primer
<400> 40
tgaaagatga gctggaggac
20
<210> 41
<211> 20
<212> DNA
<213> Artificial Sequence
<220>
<223> Description of Artificial Sequence: Synthetic primer
<400> 41
ctttcttgtc caccttggtg
20
<210> 42
<211> 19
<212> DNA
<213> Artificial Sequence
<220>
<223> Description of Artificial Sequence: Synthetic primer
<400> 42
gctgtcctac agtcctcag
19
<210> 43
<211> 18
<212> DNA
<213> Artificial Sequence
<220>
<223> Description of Artificial Sequence: Synthetic primer
<400> 43
acgtgccaag catcctcg
18
<210> 44
<211> 1407
<212> DNA
<213> Artificial Sequence
<220>
<223> Description of Artificial Sequence: Synthetic polynucleotide
<400> 44
atgtacttgg gactgaacta tgtattcata gtttttctct taaatggtgt ccagagtgaa
60
gtgaagctgg aggagtctgg aggaggcttg gtgcaacctg gaggatccat gaaactctct
120
tgtgctgcct ctggattcac ttttagtgac gcctggatgg actgggtccg ccagtctcca
180
gagaaggggc ttgagtgggt tgctgaaatt agaagcaaag ctaataatca tgcaacatac
240
tatgctgagt ctgtgaatgg gaggttcacc atctcaagag atgattccaa aagtagtgtc
300
tacctgcaaa tgaacagctt aagagctgaa gacactggca tttattactg tacgtggggg
360
gaagtgttct actttgacta ctggggccaa ggcaccactc tcacagtctc ctcagcctcc
420
accaagggcc catcggtctt ccccctggca ccctcctcca agagcacctc tgggggcaca
480
gcggccctgg gctgcctggt caaggactac ttccccgaac cggtgacggt gtcgtggaac
540
tcaggcgccc tgaccagcgg cgtgcacacc ttcccggctg tcctacagtc ctcaggactc
600
tactccctca gcagcgtggt gaccgtgccc tccagcagct tgggcaccca gacctacatc
660
tgcaacgtga atcacaagcc cagcaacacc aaggtggaca agaaagttga gcccaaatct
720
tgtgacaaaa ctcacacatg cccaccgtgc ccagcacctg aactcctggg gggaccgtca
780
gtcttcctct tccccccaaa acccaaggac accctcatga tctcccggac ccctgaggtc
840
acatgcgtgg tggtggacgt gagccacgaa gaccctgagg tcaagttcaa ctggtacgtg
900
gacggcgtgg aggtgcataa tgccaagaca aagccgcggg aggagcagta caacagcacg
960
taccgtgtgg tcagcgtcct caccgtcctg caccaggact ggctgaatgg caaggagtac
1020
aagtgcaagg tctccaacaa agccctccca gcccccatcg agaaaaccat ctccaaagcc
1080
aaagggcagc cccgagaacc acaggtgtac accctgcccc catcccggga tgagctgacc
1140
aagaaccagg tcagcctgac ctgcctggtc aaaggcttct atcccagcga catcgccgtg
1200
gagtgggaga gcaatgggca gccggagaac aactacaaga ccacgcctcc cgtgctggac
1260
tccgacggct ccttcttcct ctacagcaag ctcaccgtgg acaagagcag gtggcagcag
1320
gggaacgtct tctcatgctc cgtgatgcat gaggctctgc acaaccacta cacgcagaag
1380
agcctctccc tgtctccggg taaatga
1407
<210> 45
<211> 469
<212> PRT
<213> Artificial Sequence
<220>
<223> Description of Artificial Sequence: Synthetic polypeptide
<400> 45
Met Tyr Leu Gly Leu Asn Tyr Val Phe Ile Val Phe Leu Leu Asn Gly
1 5 10 15
Val Gln Ser Glu Val Lys Leu Glu Glu Ser Gly Gly Gly Leu Val Gln
                        20 25 30
Pro Gly Gly Ser Met Lys Leu Ser Cys Ala Ala Ser Gly Phe Thr Phe
                35 40 45
Ser Asp Ala Trp Met Asp Trp Val Arg Gln Ser Pro Glu Lys Gly Leu
        50 55 60
Glu Trp Val Ala Glu Ile Arg Ser Lys Ala Asn Asn His Ala Thr Tyr
65 70 75 80
Tyr Ala Glu Ser Val Asn Gly Arg Phe Thr Ile Ser Arg Asp Asp Ser
                                85 90 95
Lys Ser Ser Val Tyr Leu Gln Met Asn Ser Leu Arg Ala Glu Asp Thr
                        100 105 110
Gly Ile Tyr Tyr Cys Thr Trp Gly Glu Val Phe Tyr Phe Asp Tyr Trp
                115 120 125
Gly Gln Gly Thr Thr Leu Thr Val Ser Ser Ala Ser Thr Lys Gly Pro
        130 135 140
Ser Val Phe Pro Leu Ala Pro Ser Ser Lys Ser Thr Ser Gly Gly Thr
145 150 155 160
Ala Ala Leu Gly Cys Leu Val Lys Asp Tyr Phe Pro Glu Pro Val Thr
                                165 170 175
Val Ser Trp Asn Ser Gly Ala Leu Thr Ser Gly Val His Thr Phe Pro
                        180 185 190
Ala Val Leu Gln Ser Ser Gly Leu Tyr Ser Leu Ser Ser Val Val Thr
                195 200 205
Val Pro Ser Ser Ser Leu Gly Thr Gln Thr Tyr Ile Thr Cys Asn Val
        210 215 220
Asn His Lys Pro Ser Asn Thr Lys Val Asp Lys Lys Val Glu Pro Lys
225 230 235 240
Ser Cys Asp Lys Thr His Thr Cys Pro Pro Cys Pro Ala Pro Glu Leu
                                245 250 255
Leu Gly Gly Pro Ser Val Phe Leu Phe Pro Pro Lys Pro Lys Asp Thr
                        260 265 270
Leu Met Ile Ser Arg Thr Pro Glu Val Thr Cys Val Val Val Asp Val
                275 280 285
Ser His Glu Asp Pro Glu Val Lys Phe Asn Trp Tyr Val Asp Gly Val
        290 295 300
Glu Val His Asn Ala Lys Thr Lys Pro Arg Glu Glu Gln Tyr Asn Ser
305 310 315 320
Thr Tyr Arg Val Val Ser Val Leu Thr Val Leu His Gln Asp Trp Leu
                                325 330 335
Asn Gly Lys Glu Tyr Lys Cys Lys Val Ser Asn Lys Ala Leu Pro Ala
                        340 345 350
Pro Ile Glu Lys Thr Ile Ser Lys Ala Lys Gly Gln Pro Arg Glu Pro
                355 360 365
Gln Val Tyr Thr Leu Pro Pro Ser Arg Asp Glu Leu Thr Lys Asn Gln
        370 375 380
Val Ser Leu Thr Cys Leu Val Lys Gly Phe Tyr Pro Ser Asp Ile Ala
385 390 395 400
Val Glu Trp Glu Ser Asn Gly Gln Pro Glu Asn Asn Tyr Lys Thr Thr
                                405 410 415
Pro Pro Val Leu Asp Ser Asp Gly Ser Phe Phe Leu Tyr Ser Lys Leu
                        420 425 430
Thr Val Asp Lys Ser Arg Trp Gln Gln Gly Asn Val Phe Ser Cys Ser
                435 440 445
Val Met His Glu Ala Leu His Asn His Tyr Thr Gln Lys Ser Leu Ser
        450 455 460
Leu Ser Pro Gly Lys
465
<210> 46
<211> 702
<212> DNA
<213> Artificial Sequence
<220>
<223> Description of Artificial Sequence: Synthetic polynucleotide
<400> 46
atgagaccgt ctattcagtt cctggggctc ttgttgttct ggcttcatgg tgctcagtgt
60
gacatccaga tgacacagtc tccatcctca ctgtctgcat ctctgggagg caaagtcacc
120
atcacttgca agtcaagcca agacattaac aagtatatag cttggtacca acacaagcct
180
ggaaaaggtc ctaggctgct catacattac acatctacat tacagccagg catcccatca
240
aggttcagtg gaagtgggtc tgggagagat tattccttca gcatcagcaa cctggagcct
300
gaagatattg caacttatta ttgtctacag tatgataatc ttctcacgtt cggtgctggg
360
accaagctgg agctgaaacg aactgtggct gcaccatctg tcttcatctt cccgccatct
420
gatgagcagt tgaaatctgg aactgcctct gttgtgtgcc tgctgaataa cttctatccc
480
agagaggcca aagtacagtg gaaggtggat aacgccctcc aatcgggtaa ctcccaggag
540
agtgtcacag agcaggacag caaggacagc acctacagcc tcagcagcac cctgacgctg
600
agcaaagcag actacgagaa acacaaagtc tacgcctgcg aagtcaccca tcagggcctg
660
agctcgcccg tcacaaagag cttcaacagg ggagagtgtt ag
702
<210> 47
<211> 233
<212> PRT
<213> Artificial Sequence
<220>
<223> Description of Artificial Sequence: Synthetic polypeptide
<400> 47
Met Arg Pro Ser Ile Gln Phe Leu Gly Leu Leu Leu Phe Trp Leu His
1 5 10 15
Gly Ala Gln Cys Asp Ile Gln Met Thr Gln Ser Pro Ser Ser Leu Ser
                        20 25 30
Ala Ser Leu Gly Gly Lys Val Thr Ile Thr Cys Lys Ser Ser Gln Asp
                35 40 45
Ile Asn Lys Tyr Ile Ala Trp Tyr Gln His Lys Pro Gly Lys Gly Pro
        50 55 60
Arg Leu Leu Ile His Tyr Thr Ser Thr Leu Gln Pro Gly Ile Pro Ser
65 70 75 80
Arg Phe Ser Gly Ser Gly Ser Gly Arg Asp Tyr Ser Phe Ser Ile Ser
                                85 90 95
Asn Leu Glu Pro Glu Asp Ile Ala Thr Tyr Tyr Cys Leu Gln Tyr Asp
                        100 105 110
Asn Leu Leu Thr Phe Gly Ala Gly Thr Lys Leu Glu Leu Lys Arg Thr
                115 120 125
Val Ala Ala Pro Ser Val Phe Ile Phe Pro Pro Ser Asp Glu Gln Leu
        130 135 140
Lys Ser Gly Thr Ala Ser Val Val Cys Leu Leu Asn Asn Phe Tyr Pro
145 150 155 160
Arg Glu Ala Lys Val Gln Trp Lys Val Asp Asn Ala Leu Gln Ser Gly
                                165 170 175
Asn Ser Gln Glu Ser Val Thr Glu Gln Asp Ser Lys Asp Ser Thr Tyr
                        180 185 190
Ser Leu Ser Ser Thr Leu Thr Leu Ser Lys Ala Asp Tyr Glu Lys His
                195 200 205
Lys Val Tyr Ala Cys Glu Val Thr His Gln Gly Leu Ser Ser Pro Val
        210 215 220
Thr Lys Ser Phe Asn Arg Gly Glu Cys
225 230
```

SEQ ID NOS:39-43 are the sequences of oligonucleotides used for PCR amplification and sequencing of Ch119-43-1 heavy and light chain cDNA.

SEQ ID NO:44 provides the nucleotide sequence of the coding region of gamma-1 heavy chain in pCh119-43-1 along with the deduced amino acid sequence (SEQ ID NO:45). Amino acid residues are shown in single letter code.

SEQ ID NO:46 provides the nucleotide sequence of the coding region of kappa light chain in pCh119-43-1 along with the deduced amino acid sequence (SEQ ID NO:47). Amino acid residues are shown in single letter code.

### PD-1 Antigen Binding Proteins

The combinations, and methods and uses thereof, of the invention comprise anti-PD-1 antigen binding proteins that bind PD-1, such as antagonists molecules (such as antibodies) that block binding with a PD-1 ligand such as PD-L1 or PD-L2.

In an aspect, the equilibrium dissociation constant (KD) of the anti-PD-1 ABP of a combination of the invention, or a method or use thereof, and PD-1, preferably human PD-1, interaction is 100 nM or less, 10 nM or less, 2 nM or less or 1 nM or less. Alternatively the KD may be between 5 and 10 nM; or between 1 and 2 nM. The KD may be between 1 pM and 500 pM; or between 500 pM and 1 nM. A skilled person will appreciate that the smaller the KD numerical value, the stronger the binding. The reciprocal of KD (i.e. 1/KD) is the equilibrium association constant (KA) having units M−1. A skilled person will appreciate that the larger the KA numerical value, the stronger the binding.

The dissociation rate constant (kd) or “off-rate” describes the stability of the complex of the ABP on one hand and PD-1, preferably human PD-1 on the other hand, i.e. the fraction of complexes that decay per second. For example, a kd of 0.01 s−1 equates to 1% of the complexes decaying per second. In an embodiment, the dissociation rate constant (kd) is 1×10-3 s−1 or less, 1×10-4 s−1 or less, 1×10-5 s−1 or less, or 1×10-6 s−1 or less. The kd may be between 1×10-5 s−1 and 1×10-4 s−1; or between 1×10-4 s−1 and 1×10-3 s−1.

Competition between an anti-PD-1 ABP of a combination of the invention, or a method or use thereof, and a reference antibody, e.g. for binding PD-1, an epitope of PD-1, or a fragment of the PD-1, may be determined by competition ELISA, FMAT or Biacore. In one aspect, the competition assay is carried out by Biacore. There are several possible reasons for this competition: the two proteins may bind to the same or overlapping epitopes, there may be steric inhibition of binding, or binding of the first protein may induce a conformational change in the antigen that prevents or reduces binding of the second protein.

“Binding fragments” as used herein means a portion or fragment of the ABPs of a combination of the invention, or a method or use thereof, that include the antigen-binding site and are capable of binding PD-1 as defined herein, e.g. but not limited to capable of binding to the same epitope of the parent or full length antibody.

ABPs that bind human PD-1 receptor are provided herein (i.e. an anti-PD-1 ABP, sometimes referred to herein as an “anti-PD-1 ABP” or an “anti-PD-1 antibody” and/or other variations of the same). These antibodies are useful in the treatment or prevention of acute or chronic diseases or conditions whose pathology involves PD-1 signalling. In one aspect, an antigen binding protein, or isolated human antibody or functional fragment of such protein or antibody, that binds to human PD-1 and is effective as a cancer treatment or treatment against disease is described, for example in combination with another compound such as an anti-OX40 ABP, suitably an agonist anti-OX40 ABP. Any of the antigen binding proteins or antibodies disclosed herein may be used as a medicament. Any one or more of the antigen binding proteins or antibodies may be used in the methods or compositions to treat cancer, e.g. those disclosed herein.

The isolated antibodies as described herein bind to human PD-1, and may bind to human PD-1 encoded by the gene Pdcd1, or genes or cDNA sequences having 90 percent homology or 90 percent identity thereto. The complete hPD-1 mRNA sequence can be found under GenBank Accession No. U64863. The protein sequence for human PD-1 can be found at GenBank Accession No. AAC51773.

Antigen binding proteins and antibodies that bind and/or modulate PD-1 are known in the art. Exemplary anti-PD-1 ABPs of a combination of the invention, or a method or use thereof, are disclosed, for example in U.S. Pat. Nos. 8,354,509; 8,900,587; 8,008,449, each of which is incorporated by reference in its entirety herein (To the extent any definitions conflict, this instant application controls). PD-1 antibodies and methods of using in treatment of disease are described in U.S. Pat. No. 7,595,048; U.S. Pat. No. 8,168,179; U.S. Pat. No. 8,728,474; U.S. Pat. No. 7,722,868; U.S. Pat. No. 8,008,449; U.S. Pat. No. 7,488,802; U.S. Pat. No. 7,521,051; U.S. Pat. No. 8,088,905; U.S. Pat. No. 8,168,757; U.S. Pat. No. 8,354,509; and US Publication Nos. US20110171220; US20110171215; and US20110271358. Combinations of CTLA-4 and PD-1 antibodies are described in U.S. Pat. No. 9,084,776.

In another embodiment, further embodiment, any mouse or chimeric sequences of any anti-PD-1 ABP of a combination of the invention, or a method or use thereof, are engineered to make a humanized antibody.

In another embodiment, the anti-PD-1 ABP of a combination of the invention, or a method or use thereof, comprises one or more (e.g. all) of the CDRs or VH or VL or HC (heavy chain) or LC (light chain) sequences of pembrolizumab, or sequences with at least 90% (e.g., 90, 91, 92, 93, 94, 95, 96, 97, 98, or 99%) identity thereto.

The HC and LC CDRs of permolizumab are provided below. In one embodiment, the anti-PD-1 ABP of a combination of the invention, or a method or use thereof, comprises: (a) a heavy chain variable region CDR1 of pembrolizumab; (b) a heavy chain variable region CDR2 of pembrolizumab; (c) a heavy chain variable region CDR3 of pembrolizumab; (d) a light chain variable region CDR1 of pembrolizumab; (e) a light chain variable region CDR2 of pembrolizumab; and (f) a light chain variable region CDR3 of pembrolizumab.

In another embodiment, the anti-PD-1 of a combination of the invention, or a method or use thereof, comprises: a heavy chain variable region CDR1 of pembrolizumab; a heavy chain variable region CDR2 of pembrolizumab and/or a heavy chain variable region CDR3 of pembrolizumab.

In another embodiment, the anti-PD-1 of a combination of the invention, or a method or use thereof, comprises: a light chain variable region CDR1 of pembrolizumab; a light chain variable region CDR2 of pembrolizumab and/or a light chain variable region CDR3 of pembrolizumab.

In another embodiment, the anti-PD-1 ABP of a combination of the invention, or a method or use thereof, comprises: a light chain variable region (“VL”) of pembrolizumab, or an amino acid sequence with at least 90% (e.g., 90, 91, 92, 93, 94, 95, 96, 97, 98, or 99%) identity to the amino acid sequence of the VL of pembrolizumab.

In another embodiment, the anti-PD-1 ABP of a combination of the invention, or a method or use thereof, comprises a heavy chain variable region (“VH”) of pembrolizumab, or an amino acid sequence with at least 90% (e.g., 90, 91, 92, 93, 94, 95, 96, 97, 98, or 99%) identity to the amino acid sequence of the VH of pembrolizumab.

In another embodiment, the anti-PD-1 ABP of a combination of the invention, or a method or use thereof, comprises: a light chain variable region (“VL”) of pembrolizumab, or an amino acid sequence with at least 90% (e.g., 90, 91, 92, 93, 94, 95, 96, 97, 98, or 99%) identity to the amino acid sequence of the VL of pembrolizumab and the anti-PD-1 ABP of a combination of the invention, or a method or use thereof, comprises a heavy chain variable region (“VH”) of pembrolizumab, or an amino acid sequence with at least 90% (e.g., 90, 91, 92, 93, 94, 95, 96, 97, 98, or 99%) identity to the amino acid sequence of the VH of pembrolizumab.

In another embodiment, the anti-PD-1 ABP of a combination of the invention, or a method or use thereof, comprises: a light chain (“LC”) of pembrolizumab, or an amino acid sequence with at least 90% (e.g., 90, 91, 92, 93, 94, 95, 96, 97, 98, or 99%) identity to the amino acid sequence of the LC of pembrolizumab.

In another embodiment, the anti-PD-1 ABP of a combination of the invention, or a method or use thereof, comprises a heavy chain (“HC”) of pembrolizumab, or an amino acid sequence with at least 90% (e.g., 90, 91, 92, 93, 94, 95, 96, 97, 98, or 99%) identity to the amino acid sequence of the HC of pembrolizumab.

In another embodiment, the anti-PD-1 ABP of a combination of the invention, or a method or use thereof, comprises: a light chain (“LC”) of pembrolizumab, or an amino acid sequence with at least 90% (e.g., 90, 91, 92, 93, 94, 95, 96, 97, 98, or 99%) identity to the amino acid sequence of the LC of pembrolizumab and the anti-PD-1 ABP of a combination of the invention, or a method or use thereof, comprises a heavy chain (“HC”) of pembrolizumab, or an amino acid sequence with at least 90% (e.g., 90, 91, 92, 93, 94, 95, 96, 97, 98, or 99%) identity to the amino acid sequence of the HC of pembrolizumab.

Another embodiment of a combination of the invention, or a method or use thereof, includes CDRs, VH regions, and VL regions, and antibodies and nucleic acids encoding the same as disclosed in the below Sequence Listing.

An anti-OX40 ABP (e.g., an agonist ABP, e.g. an anti-hOX40 ABP, e.g. antibody), e.g., an antibody described herein, can be used in combination with an ABP (e.g., antagonist ABP, e.g antagonist antibody) against PD-1 (e.g. human PD-1). For example, an anti-OX40 antibody can be used in combination with pembrolizumab.

While in development, pembrolizumab (KEYTRUDA®) was known as MK3475 and as lambrolizumab. Pembrolizumab (KEYTRUDA®) is a human programmed death receptor-1 (PD-1)-blocking antibody indicated for the treatment of patients with unresectable or metastatic melanoma and disease progression following ipilimumab and, if BRAF V600 mutation positive, a BRAF inhibitor. The recommended dose of pembrolizumab is 2 mg/kg administered as an intravenous infusion over 30 minutes every 3 weeks until disease progression or unacceptable toxicity.

Pembrolizumab is a humanized monoclonal antibody that blocks the interaction between PD-1 and its ligands, PD-L1 and PD-L2. Pembrolizumab is an IgG4 kappa immunoglobulin with an approximate molecular weight of 149 kDa.

Pembrolizumab for injection is a sterile, preservative-free, white to off-white lyophilized powder in single-use vials. Each vial is reconstituted and diluted for intravenous infusion. Each 2 mL of reconstituted solution contains 50 mg of pembrolizumab and is formulated in L-histidine (3.1 mg), polysorbate-80 (0.4 mg), sucrose (140 mg). May contain hydrochloric acid/sodium hydroxide to adjust pH to 5.5.

Pembrolizumab injection is a sterile, preservative-free, clear to slightly opalescent, colorless to slightly yellow solution that requires dilution for intravenous infusion. Each vial contains 100 mg of pembrolizumab in 4 mL of solution. Each 1 mL of solution contains 25 mg of pembrolizumab and is formulated in: L-histidine (1.55 mg), polysorbate 80 (0.2 mg), sucrose (70 mg), and Water for Injection, USP.

Binding of the PD-1 ligands, PD-L1 and PD-L2, to the PD-1 receptor found on T cells, inhibits T cell proliferation and cytokine production. Upregulation of PD-1 ligands occurs in some tumors and signaling through this pathway can contribute to inhibition of active T-cell immune surveillance of tumors. Pembrolizumab is a monoclonal antibody that binds to the PD-1 receptor and blocks its interaction with PD-L1 and PD-L2, releasing PD-1 pathway-mediated inhibition of the immune response, including the anti-tumor immune response. In syngeneic mouse tumor models, blocking PD-1 activity resulted in decreased tumor growth.

Pembrolizumab is described, e.g in U.S. Pat. Nos. 8,354,509 and 8,900,587.

The approved product is pembrolizumab (KEYTRUDA®) for injection, for intravenous infusion of the active ingredient pembrolizumab, available as a 50 mg lyophilized powder in a single-usevial for reconstitution. Pembrolizumab has been approved for the treatment of patients with unresectable or metastatic melanoma and disease progression following ipilimumab and, if BRAF V600 mutation positive, a BRAF inhibitor. Pembrolizumab (KEYTRUDA®) is a humanized monoclonal antibody that blocks the interaction between PD-I and its ligands, PD-LI and PD-L2. Pembrolizumab is an IgG4 kappa immunoglobulin with an approximate molecular weight of 149 kDa. The amino acid sequence for pembrolizumab is as follows, and is set forth using the same one-letter amino acid code nomenclature provided in the table at column 15 of the U.S. Pat. No. 8,354,509:

| Heavy Chain of pembrolizumab: | |
|---|---|
| (SEQ ID NO: 50) | |
| QVQLVQSGVE VKKPGASVKV SCKASGYTFT NYYMYWVRQA PGQGLEWMGG | 50 |
| INPSNGGTNF NEKFKNRVTL TTDSSTTTAY MELKSLQFDD TAVYYCARRD | 100 |
| YRFDMGFDYW GQGTTVTVSS ASTKGPSVFP LAPCSRSTSE STAALGCLVK | 150 |
| DYFPEPVTVS WNSGALTSGV HTFPAVLQSS GLYSLSSVVT VPSSSLGTKT | 200 |
| YTCNVDHKPS NTKVDKRVES KYGPPCPPCP APEFLGGPSV FLFPPKPKDT | 250 |
| LMISRTPEVT CVVVDVSQED PEVQFNWYVD GVEVHNAKTK PREEQFNSTY | 300 |
| VVSVLTVLH QDWLNGKEYK CKVSNKGLPS SIEKTISKAK GQPREPQVYT | 350 |
| LPPSQEEMTK NQVSLTCLVK GFYPSDIAVE WESNGQPENN YKTTPPVLDS | 400 |
| DGSFFLYSRL TVDKSRWQEG NVFSCSVMHE ALHNHYTQKS LSLSLGK | 447 |
| Light Chain of pembrolizumab: | |
| (SEQ ID NO: 51) | |
| EIVLTQSPAT LSLSPGERAT LSCRASKGVS TSGYSYLHWY QQKPGQAPRL | 50 |
| LIYLASYLES GVPARFSGSG SGTDFTLTIS SLEPEDFAVY YCQHSRDLPL | 100 |
| TFGGGTKVEI KRTVAAPSVF IFPPSDEQLK SGTASVVCLL NNFYPREAKV | 150 |
| QWKVDNALQS GNSQESVTEQ DSKDSTYSLS STLTLSKADY EKHKVYACEV | 200 |
| THQGLSSPVT KSFNRGEC | 218 |
| Heavy Chain Variable Region of pembrolizumab: | |
| (SEQ ID NO: 52) | |
| QVQLVQSGVE VKKPGASVKV SCKASGYTFT NYYMYWVRQA PGQGLEWMGG | 50 |
| INPSNGGTNF NEKFKNRVTL TTDSSTTTAY MELKSLQFDD TAVYYCARRD | 100 |
| YRFDMGFDYW GQGTTVTVSS | |
| Light Chain Variable Region of pembrolizumab: | |
| (SEQ ID NO: 53) | |
| EIVLTQSPAT LSLSPGERAT LSCRASKGVS TSGYSYLHWY QQKPGQAPRL | 50 |
| LIYLASYLES GVPARFSGSG SGTDFTLTIS SLEPEDFAVY YCQHSRDLPL | 100 |
| TFGGGTKVEI K | |

| CDR sequences of pembrolizumab: | |
|---|---|
| HC CDR1: | |
| (SEQ ID NO: 54) | |
| Asn Tyr Tyr Met Tyr | |
| HC CDR2: | |
| (SEQ ID NO: 55) | |
| Gly Ile Asn Pro Ser Asn Gly Gly Thr Asn Phe Asn Glu Lys Phe Lys Asn | |
| HC CDR3: | |
| (SEQ ID NO: 56) | |
| Arg Asp Tyr Arg Phe Asp Met Gly Phe Asp Tyr | |
| LC CDR1: | |
| (SEQ ID NO: 57) | |
| Arg Ala Ser Lys Gly Val Ser Thr Ser Gly Tyr Ser Tyr Leu His | |
| LC CDR2: | |
| (SEQ ID NO: 58) | |
| Leu Ala Ser Tyr Leu Glu Ser | |
| LC CDR3: | |
| (SEQ ID NO: 59) | |
| Gln His Ser Arg Asp Leu Pro Leu Thr | |
| Sequence Listing from U.S. Pat. No. 8,354,509 | |
| <210> | 60 |
| <211> | 435 |
| <212> | DNA |
| <213> | Artificial Sequence |
| <220> | |
| <223> | hPD-1.08A heavy chain variable region |
| <400> | |

| | |
|---|---|
| atgrgatgga gctgtatcat kctctttttg gtagcaacag ctacaggtgt ccactcccag | 60 |
| gtccaactgc agcagcctgg ggctgaactg gtgaagcctg gggcttcagt gaagttgtcc | 120 |
| tgcaaggcct ctggctacac cttcaccagt tattatctgt actggatgaa acagaggcct | 180 |
| ggacaaggcc ttgagtggat tgggggggtt aatcctagta atggtggtac taacttcagt | 240 |
| gagaagttca agagcaaggc cacactgact gtagacaaat cctccagcac agcctacatg | 300 |
| caactcagca gcctgacatc tgaggactct gcggtctatt actgtacaag aagggattct | 360 |
| aactacgacg ggggctttga ctactggggc caaggcacta ctctcacagt ctcctcagcc | 420 |
| aaaacgacac cccca | 435 |

| | |
|---|---|
| <210> | 61 |
| <211> | 453 |
| <212> | DNA |
| <213> | Artificial Sequence |
| <220> | |
| <223> | hPD-1.08A light chain variable region |
| <400> | |

| | |
|---|---|
| atggagacag acacactcct gctatgggta ctgctgctct gggttccagg ttccactggt | 60 |
| gacattgtgc tgacacagtc tcctacttcc ttagctgtat ctctggggca gagggccacc | 120 |
| atctcatgca gggccagcaa aagtgtcagt acatctggct ttagttattt gcactggtac | 180 |
| caacagaaac caggacagcc acccaaactc ctcatctttc ttgcatccaa cctagagtct | 240 |
| ggggtccctg ccaggttcag tggcagtggg tctgggacag acttcaccct caacatccat | 300 |
| cctgtggagg aggaggacgc tgcaacctat tattgtcagc acagttggga gcttccgctc | 360 |
| acgttcggtg ctgggaccaa gctggagctg aaacgggctg atgctgcacc aactgtatcc | 420 |
| atcttcccac catccagtaa gcttgggaag ggc | 453 |

| | |
|---|---|
| <210> | 62 |
| <211> | 464 |
| <212> | DNA |
| <213> | Artificial Sequence |
| <220> | |
| <223> | hPD-1.09A heavy chain variable region |
| <400> | |

| | |
|---|---|
| atgraatgca gctgggttat yctctttttg gtagcaacag ctacaggtgt ccactcccag | 60 |
| gtccaactgc agcagcctgg ggctgaactg gtgaagcctg ggacttcagt gaagttgtcc | 120 |
| tgcaaggctt ctggctacac cttcaccaac tactatatgt actgggtgaa gcagaggcct | 180 |
| ggacaaggcc ttgagtggat tggggggatt aatcctagca atggtggtac taacttcaat | 240 |
| gagaagttca agaacaaggc cacactgact gtagacagtt cctccagcac aacctacatg | 300 |
| caactcagca gcctgacatc tgaggactct gcggtctatt actgtacaag aagggattat | 360 |
| aggttcgaca tgggctttga ctactggggc caaggcacca ctctcacagt ctcctcagcc | 420 |
| aaaacgacac ccccatccgt ytatcccbtg gcccctggaa gctt | 464 |

| | |
|---|---|
| <210> | 63 |
| <211> | 438 |
| <212> | DNA |
| <213> | Artificial Sequence |
| <220> | |
| <223> | hPD-1.09A light chain variable region |
| <400> | |

| | |
|---|---|
| atggagwcag acacactsct gytatgggta ctgctgctct gggttccagg ttccactggc | 60 |
| gacattgtgc tgacacagtc tcctgcttcc ttagctgtat ctctgggaca gagggccgcc | 120 |
| atctcatgca gggccagcaa aggtgtcagt acatctggct atagttattt gcactggtac | 180 |
| caacagaaac caggacagtc acccaaactc ctcatctatc ttgcatccta cctagaatct | 240 |
| ggggtccctg ccaggttcag tggcagtggg tctgggacag acttcaccct caacatccat | 300 |
| cctgtggagg aggaggatgc tgcaacctat tactgtcagc acagtaggga ccttccgctc | 360 |
| acgttcggta ctgggaccaa gctggagctg aaacgggctg atgctgcacc aactgtatcc | 420 |
| atcttcccac catccagt | 438 |

| <210> | 64 |
|---|---|
| <211> | 122 |
| <212> | PRT |
| <213> | Artificial Sequence |
| <220> | |
| <223> | hPD-1.08A heavy chain variable region |
| <400> | |
| Gln Val Gln Leu Gln Gln Pro Gly Ala Glu Leu Val Lys Pro Gly Ala | |
| 1 5 10 15 | |
| Ser Val Lys Leu Ser Cys Lys Ala Ser Gly Tyr Thr Phe Thr Ser Tyr | |
| 20 25 30 | |
| Tyr Leu Tyr Trp Met Lys Gln Arg Pro Gly Gln Gly Leu Glu Trp Ile | |
| 35 40 45 | |
| Gly Gly Val Asn Pro Ser Asn Gly Gly Thr Asn Phe Ser Glu Lys Phe | |
| 50 55 60 | |
| Lys Ser Lys Ala Thr Leu Thr Val Asp Lys Ser Ser Ser Thr Ala Tyr | |
| 65 70 75 80 | |
| Met Gln Leu Ser Ser Leu Thr Ser Glu Asp Ser Ala Val Tyr Tyr Cys | |
| 85 90 95 | |
| Thr Arg Arg Asp Ser Asn Tyr Asp Gly Gly Phe Asp Tyr Trp Gly Gln | |
| 100 105 110 | |
| Gly Thr Thr Leu Thr Val Ser Ser Ala Lys | |
| 115 120 | |
| <210> | 65 |
| <211> | 111 |
| <212> | PRT |
| <213> | Artificial Sequence |
| <220> | |
| <223> | hPD-1.08A light chain variable region |
| <400> | |
| Asp Ile Val Leu Thr Gln Ser Pro Thr Ser Leu Ala Val Ser Leu Gly | |
| 1 5 10 15 | |
| Gln Arg Ala Thr Ile Ser Cys Arg Ala Ser Lys Ser Val Ser Thr Ser | |
| 20 25 30 | |
| Gly Phe Ser Tyr Leu His Trp Tyr Gln Gln Lys Pro Gly Gln Pro Pro | |
| 35 40 45 | |
| Lys Leu Leu Ile Phe Leu Ala Ser Asn Leu Glu Ser Gly Val Pro Ala | |
| 50 55 60 | |
| Arg Phe Ser Gly Ser Gly Ser Gly Thr Asp Phe Thr Leu Asn Ile His | |
| 65 70 75 80 | |
| Pro Val Glu Glu Glu Asp Ala Ala Thr Tyr Tyr Cys Gln His Ser Trp | |
| 85 90 95 | |
| Glu Leu Pro Leu Thr Phe Gly Ala Gly Thr Lys Leu Glu Leu Lys | |
| 100 105 110 | |
| <210> | 66 |
| <211> | 122 |
| <212> | PRT |
| <213> | Artificial Sequence |
| <220> | |
| <223> | hPD-1.09A heavy chain variable region |
| <400> | |
| Gln Val Gln Leu Gln Gln Pro Gly Ala Glu Leu Val Lys Pro Gly Thr | |
| 1 5 10 15 | |
| Ser Val Lys Leu Ser Cys Lys Ala Ser Gly Tyr Thr Phe Thr Asn Tyr | |
| 20 25 30 | |
| Tyr Met Tyr Trp Val Lys Gln Arg Pro Gly Gln Gly Leu Glu Trp Ile | |
| 35 40 45 | |
| Gly Gly Ile Asn Pro Ser Asn Gly Gly Thr Asn Phe Asn Glu Lys Phe | |
| 50 55 60 | |
| Lys Asn Lys Ala Thr Leu Thr Val Asp Ser Ser Ser Ser Thr Thr Tyr | |
| 65 70 75 80 | |
| Met Gln Leu Ser Ser Leu Thr Ser Glu Asp Ser Ala Val Tyr Tyr Cys | |
| 85 90 95 | |
| Thr Arg Arg Asp Tyr Arg Phe Asp Met Gly Phe Asp Tyr Trp Gly Gln | |
| 100 105 110 | |
| Gly Thr Thr Leu Thr Val Ser Ser Ala Lys | |
| 115 120 | |
| <210> | 67 |
| <211> | 111 |
| <212> | PRT |
| <213> | Artificial Sequence |
| <220> | |
| <223> | hPD-1.09A light chain variable region |
| <400> | |
| Asp Ile Val Leu Thr Gln Ser Pro Ala Ser Leu Ala Val Ser Leu Gly | |
| 1 5 10 15 | |
| Gln Arg Ala Ala Ile Ser Cys Arg Ala Ser Lys Gly Val Ser Thr Ser | |
| 20 25 30 | |
| Gly Tyr Ser Tyr Leu His Trp Tyr Gln Gln Lys Pro Gly Gln Ser Pro | |
| 35 40 45 | |
| Lys Leu Leu Ile Tyr Leu Ala Ser Tyr Leu Glu Ser Gly Val Pro Ala | |
| 50 55 60 | |
| Arg Phe Ser Gly Ser Gly Ser Gly Thr Asp Phe Thr Leu Asn Ile His | |
| 65 70 75 80 | |
| Pro Val Glu Glu Glu Asp Ala Ala Thr Tyr Tyr Cys Gln His Ser Arg | |
| 85 90 95 | |
| Asp Leu Pro Leu Thr Phe Gly Thr Gly Thr Lys Leu Glu Leu Lys | |
| 100 105 110 | |
| <210> | 68 |
| <211> | 15 |
| <212> | PRT |
| <213> | Artificial Sequence |
| <220> | |
| <223> | hPD-1.08A light chain CDR1 |
| <400> | |
| Arg Ala Ser Lys Ser Val Ser Thr Ser Gly Phe Ser Tyr Leu His | |
| 1 5 10 15 | |
| <210> | 69 |
| <211> | 7 |
| <212> | PRT |
| <213> | Artificial Sequence |
| <220> | |
| <223> | hPD-1.08A light chain CDR2 |
| <400> | |
| Leu Ala Ser Asn Leu Glu Ser | |
| 1 5 | |
| <210> | 70 |
| <211> | 9 |
| <212> | PRT |
| <213> | Artificial Sequence |
| <220> | |
| <223> | hPD-1-08A light chain CDR3 |
| <400> | |
| Gln His Ser Trp Glu Leu Pro Leu Thr | |
| 1 5 | |
| <210> | 71 |
| <211> | 5 |
| <212> | PRT |
| <213> | Artificial Sequence |
| <220> | |
| <223> | hPD-1.08A heavy chain CDR1 |
| <400> | |
| Ser Tyr Tyr Leu Tyr | |
| 1 5 | |
| <210> | 72 |
| <211> | 17 |
| <212> | PRT |
| <213> | Artificial Sequence |
| <220> | |
| <223> | hPD-1.08A heavy chain CDR2 |
| <400> | |
| Gly Val Asn Pro Ser Asn Gly Gly Thr Asn Phe Ser Glu Lys Phe Lys | |
| 1 5 10 15 | |
| Ser | |
| <210> | 73 |
| <211> | 11 |
| <212> | PRT |
| <213> | Artificial Sequence |
| <220> | |
| <223> | hPD-1.08A heavy chain CDR3 |
| <400> | |
| Arg Asp Ser Asn Tyr Asp Gly Gly Phe Asp Tyr | |
| 1 5 10 | |
| <210> | 74 |
| <211> | 15 |
| <212> | PRT |
| <213> | Artificial Sequence |
| <220> | |
| <223> | hPD-1.09A light chain CDR1 |
| <400> | |
| Arg Ala Ser Lys Gly Val Ser Thr Ser Gly Tyr Ser Tyr Leu His | |
| 1 5 10 15 | |
| <210> | 75 |
| <211> | 7 |
| <212> | PRT |
| <213> | Artificial Sequence |
| <220> | |
| <223> | hPD-1.09A light chain CDR2 |
| <400> | |
| Leu Ala Ser Tyr Leu Glu Ser | |
| 1 5 | |
| <210> | 76 |
| <211> | 9 |
| <212> | PRT |
| <213> | Artificial Sequence |
| <220> | |
| <223> | hPD-1.09A light chain CDR3 |
| <400> | |
| Gln His Ser Arg Asp Leu Pro Leu Thr | |
| 1 5 | |
| <210> | 77 |
| <211> | 5 |
| <212> | PRT |
| <213> | Artificial Sequence |
| <220> | |
| <223> | hPD-1.09A heavy chain CDR1 |
| <400> | |
| Asn Tyr Tyr Met Tyr | |
| 1 5 | |
| <210> | 78 |
| <211> | 17 |
| <212> | PRT |
| <213> | Artificial Sequence |
| <220> | |
| <223> | hPD-1.09A heavy chain CDR2 |
| <400> | |
| Gly Ile Asn Pro Ser Asn Gly Gly Thr Asn Phe Asn Glu Lys Phe Lys | |
| 1 5 10 15 | |
| Asn | |
| <210> | 79 |
| <211> | 11 |
| <212> | PRT |
| <213> | Artificial Sequence |
| <220> | |
| <223> | hPD-1.09A heavy chain CDR3 |
| <400> | |
| Arg Asp Tyr Arg Phe Asp Met Gly Phe Asp Tyr | |
| 1 5 10 | |
| <210> | 80 |
| <211> | 417 |
| <212> | DNA |
| <213> | Artificial Sequence |
| <220> | |
| <223> | 109A-H heavy chain variable region |
| <220> | |
| <221> | sig_peptide |
| <222> | (1)..(57) |
| <400> | |

| | |
|---|---|
| atggactgga cctggagcat ccttttcttg gtggcagcac caacaggagc ccactcccaa | 60 |
| gtgcagctgg tgcagtctgg agttgaagtg aagaagcccg gggcctcagt gaaggtctcc | 120 |
| tgcaaggctt ctggctacac ctttaccaac tactatatgt actgggtgcg acaggcccct | 180 |
| ggacaagggc ttgagtggat gggagggatt aatcctagca atggtggtac taacttcaat | 240 |
| gagaagttca agaacagagt caccttgacc acagactcat ccacgaccac agcctacatg | 300 |
| gaactgaaga gcctgcaatt tgacgacacg gccgtttatt actgtgcgag aagggattat | 360 |
| aggttcgaca tgggctttga ctactggggc caagggacca cggtcaccgt ctcgagc | 417 |

| | |
|---|---|
| <210> | 81 |
| <211> | 417 |
| <212> | DNA |
| <213> | Artificial Sequence |
| <220> | |
| <223> | Codon optimized 109A-H heavy chain variable region |
| <220> | |
| <221> | sig_peptide |
| <222> | (1)..(57) |
| <400> | |

| | |
|---|---|
| atggactgga cctggtctat cctgttcctg gtggccgctc ctaccggcgc tcactcccag | 60 |
| gtgcagctgg tgcagtccgg cgtggaggtg aagaagcctg gcgcctccgt caaggtgtcc | 120 |
| tgcaaggcct ccggctacac cttcaccaac tactacatgt actgggtgcg gcaggctccc | 180 |
| ggccagggac tggagtggat gggcggcatc aacccttcca acggcggcac caacttcaac | 240 |
| gagaagttca agaaccgggt gaccctgacc accgactcct ccaccaccac cgcctacatg | 300 |
| gagctgaagt ccctgcagtt cgacgacacc gccgtgtact actgcgccag gcgggactac | 360 |
| cggttcgaca tgggcttcga ctactggggc cagggcacca ccgtgaccgt gtcctcc | 417 |

| | |
|---|---|
| <210> | 82 |
| <211> | 1398 |
| <212> | DNA |
| <213> | Artificial Sequence |
| <220> | |
| <223> | Codon optimized 409A-H heavy chain full length |
| <220> | |
| <221> | sig_peptide |
| <222> | (1)..(57) |
| <400> | |

| | |
|---|---|
| atggccgtgc tgggcctgct gttctgcctg gtgaccttcc cttcctgcgt gctgtcccag | 60 |
| gtgcagctgg tgcagtccgg cgtggaggtg aagaagcctg gcgcctccgt caaggtgtcc | 120 |
| tgtaaggcct ccggctacac cttcaccaac tactacatgt actgggtgcg gcaggcccca | 180 |
| ggccagggac tggagtggat gggcggcatc aacccttcca acggcggcac caacttcaac | 240 |
| gagaagttca agaaccgggt gaccctgacc accgactcct ccaccacaac cgcctacatg | 300 |
| gaactgaagt ccctgcagtt cgacgacacc gccgtgtact actgcgccag gcgggactac | 360 |
| cggttcgaca tgggcttcga ctactggggc cagggcacca ccgtgaccgt gtcctccgct | 420 |
| agcaccaagg gcccttccgt gttccctctg gccccttgct cccggtccac ctccgagtcc | 480 |
| accgccgctc tgggctgtct ggtgaaggac tacttccctg agcctgtgac cgtgagctgg | 540 |
| aactctggcg ccctgacctc cggcgtgcac accttccctg ccgtgctgca gtcctccggc | 600 |
| ctgtactccc tgtcctccgt ggtgaccgtg ccttcctcct ccctgggcac caagacctac | 660 |
| acctgcaacg tggaccacaa gccttccaac accaaggtgg acaagcgggt ggagtccaag | 720 |
| tacggccctc cttgccctcc ctgccctgcc cctgagttcc tgggcggacc ctccgtgttc | 780 |
| ctgttccctc ctaagcctaa ggacaccctg atgatctccc ggacccctga ggtgacctgc | 840 |
| gtggtggtgg acgtgtccca ggaagatcct gaggtccagt tcaattggta cgtggatggc | 900 |
| gtggaggtgc acaacgccaa gaccaagcct cgggaggaac agttcaactc cacctaccgg | 960 |
| gtggtgtctg tgctgaccgt gctgcaccag gactggctga acggcaagga atacaagtgc | 1020 |
| aaggtcagca acaagggcct gccctcctcc atcgagaaaa ccatctccaa ggccaagggc | 1080 |
| cagcctcgcg agcctcaggt gtacaccctg cctcctagcc aggaagagat gaccaagaat | 1140 |
| caggtgtccc tgacatgcct ggtgaagggc ttctaccctt ccgatatcgc cgtggagtgg | 1200 |
| gagagcaacg gccagccaga gaacaactac aagaccaccc ctcctgtgct ggactccgac | 1260 |
| ggctccttct tcctgtactc caggctgacc gtggacaagt cccggtggca ggaaggcaac | 1320 |
| gtcttttcct gctccgtgat gcacgaggcc ctgcacaacc actacaccca gaagtccctg | 1380 |
| tccctgtctc tgggcaag | 1398 |

| | |
|---|---|
| <210> | 83 |
| <211> | 393 |
| <212> | DNA |
| <213> | Artificial Sequence |
| <220> | |
| <223> | K09A-L-11 light chain variable region |
| <220> | |
| <221> | sig_peptide |
| <222> | (1)..(60) |
| <400> | |

| | |
|---|---|
| atggaagccc cagctcagct tctcttcctc ctgctactct ggctcccaga taccaccgga | 60 |
| gaaattgtgt tgacacagtc tccagccacc ctgtctttgt ctccagggga aagagccacc | 120 |
| ctctcctgca gggccagcaa aggtgtcagt acatctggct atagttattt gcactggtat | 180 |
| caacagaaac ctggccaggc tcccaggctc ctcatctatc ttgcatccta cctagaatct | 240 |
| ggcgtcccag ccaggttcag tggtagtggg tctgggacag acttcactct caccatcagc | 300 |
| agcctagagc ctgaagattt tgcagtttat tactgtcagc acagcaggga ccttccgctc | 360 |
| acgttcggcg gagggaccaa agtggagatc aaa | 393 |

| | |
|---|---|
| <210> | 84 |
| <211> | 393 |
| <212> | DNA |
| <213> | Artificial Sequence |
| <220> | |
| <223> | K09A-L-16 light chain variable region |
| <220> | |
| <221> | sig_peptide |
| <222> | (1)..(60) |
| <400> | |

| | |
|---|---|
| atggaaaccc cagcgcagct tctcttcctc ctgctactct ggctcccaga taccaccgga | 60 |
| gaaattgtgc tgactcagtc tccactctcc ctgcccgtca cccctggaga gccggcctcc | 120 |
| atctcctgca gggccagcaa aggtgtcagt acatctggct atagttattt gcattggtac | 180 |
| ctccagaagc cagggcagtc tccacagctc ctgatctatc ttgcatccta cctagaatct | 240 |
| ggggtccctg acaggttcag tggcagtgga tcaggcacag attttacact gaaaatcagc | 300 |
| agagtggagg ctgaggatgt tggggtttat tactgccagc atagtaggga ccttccgctc | 360 |
| acgtttggcc aggggaccaa gctggagatc aaa | 393 |

| | |
|---|---|
| <210> | 85 |
| <211> | 393 |
| <212> | DNA |
| <213> | Artificial Sequence |
| <220> | |
| <223> | K09A-L-17 light chain variable region |
| <220> | |
| <221> | sig_peptide |
| <222> | (1)..(60) |
| <400> | |

| | |
|---|---|
| atgaggctcc ctgctcagct cctggggctg ctaatgctct gggtctctgg atccagtggg | 60 |
| gatattgtga tgacccagac tccactctcc ctgcccgtca cccctggaga gccggcctcc | 120 |
| atctcctgca gggccagcaa aggtgtcagt acatctggct atagttattt gcattggtat | 180 |
| ctgcagaagc cagggcagtc tccacagctc ctgatctatc ttgcatccta cctagaatct | 240 |
| ggagtcccag acaggttcag tggcagtggg tcaggcactg ctttcacact gaaaatcagc | 300 |
| agggtggagg ctgaggatgt tggactttat tactgccagc atagtaggga ccttccgctc | 360 |
| acgtttggcc aggggaccaa gctggagatc aaa | 393 |

| | |
|---|---|
| <210> | 86 |
| <211> | 390 |
| <212> | DNA |
| <213> | Artificial Sequence |
| <220> | |
| <223> | Codon optimized K09A-L-11 light chain variable region |
| <220> | |
| <221> | sig_peptide |
| <222> | (1)..(57) |
| <400> | |

| | |
|---|---|
| atggcccctg tgcagctgct gggcctgctg gtgctgttcc tgcctgccat gcggtgcgag | 60 |
| atcgtgctga cccagtcccc tgccaccctg tccctgagcc ctggcgagcg ggctaccctg | 120 |
| agctgcagag cctccaaggg cgtgtccacc tccggctact cctacctgca ctggtatcag | 180 |
| cagaagccag gccaggcccc tcggctgctg atctacctgg cctcctacct ggagtccggc | 240 |
| gtgcctgccc ggttctccgg ctccggaagc ggcaccgact tcaccctgac catctcctcc | 300 |
| ctggagcctg aggacttcgc cgtgtactac tgccagcact cccgggacct gcctctgacc | 360 |
| tttggcggcg gaacaaaggt ggagatcaag | 390 |

| | |
|---|---|
| <210> | 87 |
| <211> | 390 |
| <212> | DNA |
| <213> | Artificial Sequence |
| <220> | |
| <223> | Codon optimized K09A-L-16 light chain variable region |
| <220> | |
| <221> | sig_peptide |
| <222> | (1)..(57) |
| <400> | |

| | |
|---|---|
| atggcccctg tgcagctgct gggcctgctg gtgctgttcc tgcctgccat gcggtgcgag | 60 |
| atcgtgctga cccagtcccc tctgtccctg cctgtgaccc ctggcgagcc tgcctccatc | 120 |
| tcctgccggg cctccaaggg cgtgtccacc tccggctact cctacctgca ctggtatctg | 180 |
| cagaagcctg gccagtcccc ccagctgctg atctacctgg cctcctacct ggagtccggc | 240 |
| gtgcctgacc ggttctccgg ctccggcagc ggcaccgact tcaccctgaa gatctcccgg | 300 |
| gtggaggccg aggacgtggg cgtgtactac tgccagcact cccgggacct gcctctgacc | 360 |
| ttcggccagg gcaccaagct ggagatcaag | 390 |

| | |
|---|---|
| <210> | 88 |
| <211> | 390 |
| <212> | DNA |
| <213> | Artificial Sequence |
| <220> | |
| <223> | Codon optimized K09A-L-17 light chain variable region |
| <220> | |
| <221> | sig_peptide |
| <222> | (1)..(57) |
| <400> | |

| | |
|---|---|
| atggcccctg tgcagctgct gggcctgctg gtgctgttcc tgcctgccat gcggtgcgac | 60 |
| atcgtgatga cccagacccc tctgtccctg cctgtgaccc ctggcgagcc tgcctccatc | 120 |
| tcctgccggg cctccaaggg cgtgtccacc tccggctact cctacctgca ctggtatctg | 180 |
| cagaagcctg gccagtcccc tcagctgctg atctacctgg cctcctacct ggagtccggc | 240 |
| gtgcctgacc ggttctccgg ctccggaagc ggcaccgctt ttaccctgaa gatctccaga | 300 |
| gtggaggccg aggacgtggg cctgtactac tgccagcact cccgggacct gcctctgacc | 360 |
| ttcggccagg gcaccaagct ggagatcaag | 390 |

| <210> | 89 |
|---|---|
| <211> | 139 |
| <212> | PRT |
| <213> | Artificial Sequence |
| <220> | |
| <223> | 109A-H heavy chain variable region |
| <220> | |
| <221> | sig_peptide |
| <222> | (1)..(19) |
| <400> | |
| Met Asp Trp Thr Trp Ser Ile Leu Phe Leu Val Ala Ala Pro Thr Gly | |
| 1 5 10 15 | |
| Ala His Ser Gln Val Gln Leu Val Gln Ser Gly Val Glu Val Lys Lys | |
| 20 25 30 | |
| Pro Gly Ala Ser Val Lys Val Ser Cys Lys Ala Ser Gly Tyr Thr Phe | |
| 35 40 45 | |
| Thr Asn Tyr Tyr Met Tyr Trp Val Arg Gln Ala Pro Gly Gln Gly Leu | |
| 50 55 60 | |
| Glu Trp Met Gly Gly Ile Asn Pro Ser Asn Gly Gly Thr Asn Phe Asn | |
| 65 70 75 80 | |
| Glu Lys Phe Lys Asn Arg Val Thr Leu Thr Thr Asp Ser Ser Thr Thr | |
| 85 90 95 | |
| Thr Ala Tyr Met Glu Leu Lys Ser Leu Gln Phe Asp Asp Thr Ala Val | |
| 100 105 110 | |
| Tyr Tyr Cys Ala Arg Arg Asp Tyr Arg Phe Asp Met Gly Phe Asp Tyr | |
| 115 120 125 | |
| Trp Gly Gln Gly Thr Thr Val Thr Val Ser Ser | |
| 130 135 | |
| <210> | 90 |
| <211> | 466 |
| <212> | PRT |
| <213> | Artificial Sequence |
| <220> | |
| <223> | 409A-H heavy chain full length |
| <220> | |
| <221> | sig_peptide |
| <222> | (1)..(19) |
| <400> | |
| Met Ala Val Leu Gly Leu Leu Phe Cys Leu Val Thr Phe Pro Ser Cys | |
| 1 5 10 15 | |
| Val Leu Ser Gln Val Gln Leu Val Gln Ser Gly Val Glu Val Lys Lys | |
| 20 25 30 | |
| Pro Gly Ala Ser Val Lys Val Ser Cys Lys Ala Ser Gly Tyr Thr Phe | |
| 35 40 45 | |
| Thr Asn Tyr Tyr Met Tyr Trp Val Arg Gln Ala Pro Gly Gln Gly Leu | |
| 50 55 60 | |
| Glu Trp Met Gly Gly Ile Asn Pro Ser Asn Gly Gly Thr Asn Phe Asn | |
| 65 70 75 80 | |
| Glu Lys Phe Lys Asn Arg Val Thr Leu Thr Thr Asp Ser Ser Thr Thr | |
| 85 90 95 | |
| Thr Ala Tyr Met Glu Leu Lys Ser Leu Gln Phe Asp Asp Thr Ala Val | |
| 100 105 110 | |
| Tyr Tyr Cys Ala Arg Arg Asp Tyr Arg Phe Asp Met Gly Phe Asp Tyr | |
| 115 120 125 | |
| Trp Gly Gln Gly Thr Thr Val Thr Val Ser Ser Ala Ser Thr Lys Gly | |
| 130 135 140 | |
| Pro Ser Val Phe Pro Leu Ala Pro Cys Ser Arg Ser Thr Ser Glu Ser | |
| 145 150 155 160 | |
| Thr Ala Ala Leu Gly Cys Leu Val Lys Asp Tyr Phe Pro Glu Pro Val | |
| 165 170 175 | |
| Thr Val Ser Trp Asn Ser Gly Ala Leu Thr Ser Gly Val His Thr Phe | |
| 180 185 190 | |
| Pro Ala Val Leu Gln Ser Ser Gly Leu Tyr Ser Leu Ser Ser Val Val | |
| 195 200 205 | |
| Thr Val Pro Ser Ser Ser Leu Gly Thr Lys Thr Tyr Thr Cys Asn Val | |
| 210 215 220 | |
| Asp His Lys Pro Ser Asn Thr Lys Val Asp Lys Arg Val Glu Ser Lys | |
| 225 230 235 240 | |
| Tyr Gly Pro Pro Cys Pro Pro Cys Pro Ala Pro Glu Phe Leu Gly Gly | |
| 245 250 255 | |
| Pro Ser Val Phe Leu Phe Pro Pro Lys Pro Lys Asp Thr Leu Met Ile | |
| 260 265 270 | |
| Ser Arg Thr Pro Glu Val Thr Cys Val Val Val Asp Val Ser Gln Glu | |
| 275 280 285 | |
| Asp Pro Glu Val Gln Phe Asn Trp Tyr Val Asp Gly Val Glu Val His | |
| 290 295 300 | |
| Asn Ala Lys Thr Lys Pro Arg Glu Glu Gln Phe Asn Ser Thr Tyr Arg | |
| 305 310 315 320 | |
| Val Val Ser Val Leu Thr Val Leu His Gln Asp Trp Leu Asn Gly Lys | |
| 325 330 335 | |
| Glu Tyr Lys Cys Lys Val Ser Asn Lys Gly Leu Pro Ser Ser Ile Glu | |
| 340 345 350 | |
| Lys Thr Ile Ser Lys Ala Lys Gly Gln Pro Arg Glu Pro Gln Val Tyr | |
| 355 360 365 | |
| Thr Leu Pro Pro Ser Gln Glu Glu Met Thr Lys Asn Gln Val Ser Leu | |
| 370 375 380 | |
| Thr Cys Leu Val Lys Gly Phe Tyr Pro Ser Asp Ile Ala Val Glu Trp | |
| 385 390 395 400 | |
| Glu Ser Asn Gly Gln Pro Glu Asn Asn Tyr Lys Thr Thr Pro Pro Val | |
| 405 410 415 | |
| Leu Asp Ser Asp Gly Ser Phe Phe Leu Tyr Ser Arg Leu Thr Val Asp | |
| 420 425 430 | |
| Lys Ser Arg Trp Gln Glu Gly Asn Val Phe Ser Cys Ser Val Met His | |
| 435 440 445 | |
| Glu Ala Leu His Asn His Tyr Thr Gln Lys Ser Leu Ser Leu Ser Leu | |
| 450 455 460 | |
| Gly Lys | |
| 465 | |
| <210> | 91 |
| <211> | 130 |
| <212> | PRT |
| <213> | Artificial Sequence |
| <220> | |
| <223> | K09A-L-11 light chain variable region |
| <220> | |
| <221> | sig_peptide |
| <222> | (1)..(19) |
| <400> | |
| Met Ala Pro Val Gln Leu Leu Gly Leu Leu Val Leu Phe Leu Pro Ala | |
| 1 5 10 15 | |
| Met Arg Cys Glu Ile Val Leu Thr Gln Ser Pro Ala Thr Leu Ser Leu | |
| 20 25 30 | |
| Ser Pro Gly Glu Arg Ala Thr Leu Ser Cys Arg Ala Ser Lys Gly Val | |
| 35 40 45 | |
| Ser Thr Ser Gly Tyr Ser Tyr Leu His Trp Tyr Gln Gln Lys Pro Gly | |
| 50 55 60 | |
| Gln Ala Pro Arg Leu Leu Ile Tyr Leu Ala Ser Tyr Leu Glu Ser Gly | |
| 65 70 75 80 | |
| Val Pro Ala Arg Phe Ser Gly Ser Gly Ser Gly Thr Asp Phe Thr Leu | |
| 85 90 95 | |
| Thr Ile Ser Ser Leu Glu Pro Glu Asp Phe Ala Val Tyr Tyr Cys Gln | |
| 100 105 110 | |
| His Ser Arg Asp Leu Pro Leu Thr Phe Gly Gly Gly Thr Lys Val Glu | |
| 115 120 125 | |
| Ile Lys | |
| 130 | |
| <210> | 92 |
| <211> | 130 |
| <212> | PRT |
| <213> | Artificial Sequence |
| <220> | |
| <223> | K09A-L-16 light chain variable region |
| <220> | |
| <221> | sig_peptide |
| <222> | (1)..(19) |
| <400> | |
| Met Ala Pro Val Gln Leu Leu Gly Leu Leu Val Leu Phe Leu Pro Ala | |
| 1 5 10 15 | |
| Met Arg Cys Glu Ile Val Leu Thr Gln Ser Pro Leu Ser Leu Pro Val | |
| 20 25 30 | |
| Thr Pro Gly Glu Pro Ala Ser Ile Ser Cys Arg Ala Ser Lys Gly Val | |
| 35 40 45 | |
| Ser Thr Ser Gly Tyr Ser Tyr Leu His Trp Tyr Leu Gln Lys Pro Gly | |
| 50 55 60 | |
| Gln Ser Pro Gln Leu Leu Ile Tyr Leu Ala Ser Tyr Leu Glu Ser Gly | |
| 65 70 75 80 | |
| Val Pro Asp Arg Phe Ser Gly Ser Gly Ser Gly Thr Asp Phe Thr Leu | |
| 85 90 95 | |
| Lys Ile Ser Arg Val Glu Ala Glu Asp Val Gly Val Tyr Tyr Cys Gln | |
| 100 105 110 | |
| His Ser Arg Asp Leu Pro Leu Thr Phe Gly Gln Gly Thr Lys Leu Glu | |
| 115 120 125 | |
| Ile Lys | |
| 130 | |
| <210> | 93 |
| <211> | 130 |
| <212> | PRT |
| <213> | Artificial Sequence |
| <220> | |
| <223> | K09A-L-17 light chain variable region |
| <220> | |
| <221> | sig_peptide |
| <222> | (1)..(19) |
| <400> | |
| Met Ala Pro Val Gln Leu Leu Gly Leu Leu Val Leu Phe Leu Pro Ala | |
| 1 5 10 15 | |
| Met Arg Cys Asp Ile Val Met Thr Gln Thr Pro Leu Ser Leu Pro Val | |
| 20 25 30 | |
| Thr Pro Gly Glu Pro Ala Ser Ile Ser Cys Arg Ala Ser Lys Gly Val | |
| 35 40 45 | |
| Ser Thr Ser Gly Tyr Ser Tyr Leu His Trp Tyr Leu Gln Lys Pro Gly | |
| 50 55 60 | |
| Gln Ser Pro Gln Leu Leu Ile Tyr Leu Ala Ser Tyr Leu Glu Ser Gly | |
| 65 70 75 80 | |
| Val Pro Asp Arg Phe Ser Gly Ser Gly Ser Gly Thr Ala Phe Thr Leu | |
| 85 90 95 | |
| Lys Ile Ser Arg Val Glu Ala Glu Asp Val Gly Leu Tyr Tyr Cys Gln | |
| 100 105 110 | |
| His Ser Arg Asp Leu Pro Leu Thr Phe Gly Gln Gly Thr Lys Leu Glu | |
| 115 120 125 | |
| Ile Lys | |
| 130 | |
| <210> | 94 |
| <211> | 469 |
| <212> | PRT |
| <213> | Artificial Sequence |
| <220> | |
| <223> | 109A-H heavy chain full length |
| <220> | |
| <221> | sig_peptide |
| <222> | (1)..(19) |
| <400> | |
| Met Ala Val Leu Gly Leu Leu Phe Cys Leu Val Thr Phe Pro Ser Cys | |
| 1 5 10 15 | |
| Val Leu Ser Gln Val Gln Leu Val Gln Ser Gly Val Glu Val Lys Lys | |
| 20 25 30 | |
| Pro Gly Ala Ser Val Lys Val Ser Cys Lys Ala Ser Gly Tyr Thr Phe | |
| 35 40 45 | |
| Thr Asn Tyr Tyr Met Tyr Trp Val Arg Gln Ala Pro Gly Gln Gly Leu | |
| 50 55 60 | |
| Glu Trp Met Gly Gly Ile Asn Pro Ser Asn Gly Gly Thr Asn Phe Asn | |
| 65 70 75 80 | |
| Glu Lys Phe Lys Asn Arg Val Thr Leu Thr Thr Asp Ser Ser Thr Thr | |
| 85 90 95 | |
| Thr Ala Tyr Met Glu Leu Lys Ser Leu Gln Phe Asp Asp Thr Ala Val | |
| 100 105 110 | |
| Tyr Tyr Cys Ala Arg Arg Asp Tyr Arg Phe Asp Met Gly Phe Asp Tyr | |
| 115 120 125 | |
| Trp Gly Gln Gly Thr Thr Val Thr Val Ser Ser Ala Ser Thr Lys Gly | |
| 130 135 140 | |
| Pro Ser Val Phe Pro Leu Ala Pro Ser Ser Lys Ser Thr Ser Gly Gly | |
| 145 150 155 160 | |
| Thr Ala Ala Leu Gly Cys Leu Val Lys Asp Tyr Phe Pro Glu Pro Val | |
| 165 170 175 | |
| Thr Val Ser Trp Asn Ser Gly Ala Leu Thr Ser Gly Val His Thr Phe | |
| 180 185 190 | |
| Pro Ala Val Leu Gln Ser Ser Gly Leu Tyr Ser Leu Ser Ser Val Val | |
| 195 200 205 | |
| Thr Val Pro Ser Ser Ser Leu Gly Thr Gln Thr Tyr Ile Cys Asn Val | |
| 210 215 220 | |
| Asn His Lys Pro Ser Asn Thr Lys Val Asp Lys Lys Val Glu Pro Lys | |
| 225 230 235 240 | |
| Ser Cys Asp Lys Thr His Thr Cys Pro Pro Cys Pro Ala Pro Glu Leu | |
| 245 250 255 | |
| Leu Gly Gly Pro Ser Val Phe Leu Phe Pro Pro Lys Pro Lys Asp Thr | |
| 260 265 270 | |
| Leu Met Ile Ser Arg Thr Pro Glu Val Thr Cys Val Val Val Asp Val | |
| 275 280 285 | |
| Ser His Glu Asp Pro Glu Val Lys Phe Asn Trp Tyr Val Asp Gly Val | |
| 290 295 300 | |
| Glu Val His Asn Ala Lys Thr Lys Pro Arg Glu Glu Gln Tyr Asn Ser | |
| 305 310 315 320 | |
| Thr Tyr Arg Val Val Ser Val Leu Thr Val Leu His Gln Asp Trp Leu | |
| 325 330 335 | |
| Asn Gly Lys Glu Tyr Lys Cys Lys Val Ser Asn Lys Ala Leu Pro Ala | |
| 340 345 350 | |
| Pro Ile Glu Lys Thr Ile Ser Lys Ala Lys Gly Gln Pro Arg Glu Pro | |
| 355 360 365 | |
| Gln Val Tyr Thr Leu Pro Pro Ser Arg Asp Glu Leu Thr Lys Asn Gln | |
| 370 375 380 | |
| Val Ser Leu Thr Cys Leu Val Lys Gly Phe Tyr Pro Ser Asp Ile Ala | |
| 385 390 395 400 | |
| Val Glu Trp Glu Ser Asn Gly Gln Pro Glu Asn Asn Tyr Lys Thr Thr | |
| 405 410 415 | |
| Pro Pro Val Leu Asp Ser Asp Gly Ser Phe Phe Leu Tyr Ser Lys Leu | |
| 420 425 430 | |
| Thr Val Asp Lys Ser Arg Trp Gln Gln Gly Asn Val Phe Ser Cys Ser | |
| 435 440 445 | |
| Val Met His Glu Ala Leu His Asn His Tyr Thr Gln Lys Ser Leu Ser | |
| 450 455 460 | |
| Leu Ser Pro Gly Lys | |
| 465 | |
| <210> | 95 |
| <211> | 237 |
| <212> | PRT |
| <213> | Artificial Sequence |
| <220> | |
| <223> | K09A-L-11 light chain full length |
| <220> | |
| <221> | sig_peptide |
| <222> | (1)..(19) |
| <400> | |
| Met Ala Pro Val Gln Leu Leu Gly Leu Leu Val Leu Phe Leu Pro Ala | |
| 1 5 10 15 | |
| Met Arg Cys Glu Ile Val Leu Thr Gln Ser Pro Ala Thr Leu Ser Leu | |
| 20 25 30 | |
| Ser Pro Gly Glu Arg Ala Thr Leu Ser Cys Arg Ala Ser Lys Gly Val | |
| 35 40 45 | |
| Ser Thr Ser Gly Tyr Ser Tyr Leu His Trp Tyr Gln Gln Lys Pro Gly | |
| 50 55 60 | |
| Gln Ala Pro Arg Leu Leu Ile Tyr Leu Ala Ser Tyr Leu Glu Ser Gly | |
| 65 70 75 80 | |
| Val Pro Ala Arg Phe Ser Gly Ser Gly Ser Gly Thr Asp Phe Thr Leu | |
| 85 90 95 | |
| Thr Ile Ser Ser Leu Glu Pro Glu Asp Phe Ala Val Tyr Tyr Cys Gln | |
| 100 105 110 | |
| His Ser Arg Asp Leu Pro Leu Thr Phe Gly Gly Gly Thr Lys Val Glu | |
| 115 120 125 | |
| Ile Lys Arg Thr Val Ala Ala Pro Ser Val Phe Ile Phe Pro Pro Ser | |
| 130 135 140 | |
| Asp Glu Gln Leu Lys Ser Gly Thr Ala Ser Val Val Cys Leu Leu Asn | |
| 145 150 155 160 | |
| Asn Phe Tyr Pro Arg Glu Ala Lys Val Gln Trp Lys Val Asp Asn Ala | |
| 165 170 175 | |
| Leu Gln Ser Gly Asn Ser Gln Glu Ser Val Thr Glu Gln Asp Ser Lys | |
| 180 185 190 | |
| Asp Ser Thr Tyr Ser Leu Ser Ser Thr Leu Thr Leu Ser Lys Ala Asp | |
| 195 200 205 | |
| Tyr Glu Lys His Lys Val Tyr Ala Cys Glu Val Thr His Gln Gly Leu | |
| 210 215 220 | |
| Ser Ser Pro Val Thr Lys Ser Phe Asn Arg Gly Glu Cys | |
| 225 230 235 | |
| <210> | 96 |
| <211> | 237 |
| <212> | PRT |
| <213> | Artificial Sequence |
| <220> | |
| <223> | K09A-L-16 light chain full length |
| <220> | |
| <221> | sig_peptide |
| <222> | (1)..(19) |
| <400> | |
| Met Ala Pro Val Gln Leu Leu Gly Leu Leu Val Leu Phe Leu Pro Ala | |
| 1 5 10 15 | |
| Met Arg Cys Glu Ile Val Leu Thr Gln Ser Pro Leu Ser Leu Pro Val | |
| 20 25 30 | |
| Thr Pro Gly Glu Pro Ala Ser Ile Ser Cys Arg Ala Ser Lys Gly Val | |
| 35 40 45 | |
| Ser Thr Ser Gly Tyr Ser Tyr Leu His Trp Tyr Leu Gln Lys Pro Gly | |
| 50 55 60 | |
| Gln Ser Pro Gln Leu Leu Ile Tyr Leu Ala Ser Tyr Leu Glu Ser Gly | |
| 65 70 75 80 | |
| Val Pro Asp Arg Phe Ser Gly Ser Gly Ser Gly Thr Asp Phe Thr Leu | |
| 85 90 95 | |
| Lys Ile Ser Arg Val Glu Ala Glu Asp Val Gly Val Tyr Tyr Cys Gln | |
| 100 105 110 | |
| His Ser Arg Asp Leu Pro Leu Thr Phe Gly Gln Gly Thr Lys Leu Glu | |
| 115 120 125 | |
| Ile Lys Arg Thr Val Ala Ala Pro Ser Val Phe Ile Phe Pro Pro Ser | |
| 130 135 140 | |
| Asp Glu Gln Leu Lys Ser Gly Thr Ala Ser Val Val Cys Leu Leu Asn | |
| 145 150 155 160 | |
| Asn Phe Tyr Pro Arg Glu Ala Lys Val Gln Trp Lys Val Asp Asn Ala | |
| 165 170 175 | |
| Leu Gln Ser Gly Asn Ser Gln Glu Ser Val Thr Glu Gln Asp Ser Lys | |
| 180 185 190 | |
| Asp Ser Thr Tyr Ser Leu Ser Ser Thr Leu Thr Leu Ser Lys Ala Asp | |
| 195 200 205 | |
| Tyr Glu Lys His Lys Val Tyr Ala Cys Glu Val Thr His Gln Gly Leu | |
| 210 215 220 | |
| Ser Ser Pro Val Thr Lys Ser Phe Asn Arg Gly Glu Cys | |
| 225 230 235 | |
| <210> | 97 |
| <211> | 237 |
| <212> | PRT |
| <213> | Artificial Sequence |
| <220> | |
| <223> | K09A-L-17 light chain full length |
| <220> | |
| <221> | sig_peptide |
| <222> | (1)..(19) |
| <400> | |

```
Met Ala Pro Val Gln Leu Leu Gly Leu Leu Val Leu Phe Leu Pro Ala
1 5 10 15
Met Arg Cys Asp Ile Val Met Thr Gln Thr Pro Leu Ser Leu Pro Val
                        20 25 30
Thr Pro Gly Glu Pro Ala Ser Ile Ser Cys Arg Ala Ser Lys Gly Val
                35 40 45
Ser Thr Ser Gly Tyr Ser Tyr Leu His Trp Tyr Leu Gln Lys Pro Gly
        50 55 60
Gln Ser Pro Gln Leu Leu Ile Tyr Leu Ala Ser Tyr Leu Glu Ser Gly
65 70 75 80
Val Pro Asp Arg Phe Ser Gly Ser Gly Ser Gly Thr Ala Phe Thr Leu
                                85 90 95
Lys Ile Ser Arg Val Glu Ala Glu Asp Val Gly Leu Tyr Tyr Cys Gln
                        100 105 110
His Ser Arg Asp Leu Pro Leu Thr Phe Gly Gln Gly Thr Lys Leu Glu
                115 120 125
Ile Lys Arg Thr Val Ala Ala Pro Ser Val Phe Ile Phe Pro Pro Ser
        130 135 140
Asp Glu Gln Leu Lys Ser Gly Thr Ala Ser Val Val Cys Leu Leu Asn
145 150 155 160
Asn Phe Tyr Pro Arg Glu Ala Lys Val Gln Trp Lys Val Asp Asn Ala
                                165 170 175
Leu Gln Ser Gly Asn Ser Gln Glu Ser Val Thr Glu Gln Asp Ser Lys
                        180 185 190
Asp Ser Thr Tyr Ser Leu Ser Ser Thr Leu Thr Leu Ser Lys Ala Asp
                195 200 205
Tyr Glu Lys His Lys Val Tyr Ala Cys Glu Val Thr His Gln Gly Leu
        210 215 220
Ser Ser Pro Val Thr Lys Ser Phe Asn Arg Gly Glu Cys
225 230 235
```

As another example, an anti-OX40 antibody can be used in combination with nivolumab (OPDIVO®). Nivolumab (OPDIVO®) is a programmed death receptor-1 (PD-1) blocking antibody indicated for the treatment of patients with:
unresectable or metastatic melanoma and disease progression following ipilimumab and, if BRAF V600 mutation positive, a BRAF inhibitor. This indication is approved under accelerated approval based on tumor response rate and durability of response. Continued approval for this indication may be contingent upon verification and description of clinical benefit in the confirmatory trials.
metastatic squamous non-small cell lung cancer with progression on or after platinum-based chemotherapy.

The recommended dose of nivolumab (OPDIVO®) is 3 mg/kg administered as an intravenous infusion over 60 minutes every 2 weeks until disease progression or unacceptable toxicity.

Binding of the PD-1 ligands, PD-L1 and PD-L2, to the PD-1 receptor found on T cells, inhibits T-cell proliferation and cytokine production. Upregulation of PD-1 ligands occurs in some tumors and signaling through this pathway can contribute to inhibition of active T-cell immune surveillance of tumors.

Nivolumab is a human immunoglobulin G4 (IgG4) monoclonal antibody that binds to the PD-1 receptor and blocks its interaction with PD-L1 and PD-L2, releasing PD-1 pathway-mediated inhibition of the immune response, including the anti-tumor immune response. In syngeneic mouse tumor models, blocking PD-1 activity resulted in decreased tumor growth.

U.S. Pat. No. 8,008,449 exemplifies seven anti-PD-1 HuMAbs: 17D8, 2D3, 4H1, 504 (also referred to herein as nivolumab or BMS-936558), 4A1 1, 7D3 and 5F4. See also U.S. Pat. No. 8,779,105. Any one of these antibodies, or the CDRs thereof (or an amino acid sequence with at least 90% (e.g., 90, 91, 92, 93, 94, 95, 96, 97, 98, or 99%) identity to any of these amino acid sequences), can be used in the compositions and methods described herein.

```
Heavy Chain of nivolumab:
(SEQ ID NO: 98)
QVQLVESGGGVVQPGRSLRLDCKASGITFSNSGMHWVRQAPGKGLEWVAV
IWYDGSKRYYADSVKGRFTISRDNSKNTLFLQMNSLRAEDTAVYYCATND
DYWGQGTLVTVSSASTKGPSVFPLAPCSRSTSESTAALGCLVKDYFPEPV
TVSWNSGALTSGVHTFPAVLQSSGLYSLSSVVTVPSSSLGTKTYTCNVDH
KPSNTKVDKRVESKYGPPCPPCPAPEFLGGPSVFLFPPKPKDTLMISRTP
EVTCVVVDVSQEDPEVQFNWYVDGVEVHNAKTKPREEQFNSTYRVVSVLT
VLHQDWLNGKEYKCKVSNKGLPSSIEKTISKAKGQPREPQVYTLPPSQEE
MTKNQVSLTCLVKGFYPSDIAVEWESNGQPENNYKTTPPVLDSDGSFFLY
SRLTVDKSRWQEGNVFSCSVMHEALHNHYTQKSLSLSLGK
Light Chain of nivolumab:
(SEQ ID NO: 99)
EIVLTQSPATLSLSPGERATLSCRASQSVSSYLAWYQQKPGQAPRLLIYD
ASNRATGIPARFSGSGSGTDFTLTISSLEPEDFAVYYCQQSSNWPRTFGQ
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC
Heavy Chain Variable region of nivolumab:
(SEQ ID NO: 100)
Gln Val Gln Leu Val Glu Ser Gly Gly Gly Val Val
1 5 10
Gln Pro Gly Arg Ser Leu Arg Leu Asp Cys Lys Ala
                15 20
Ser Gly Ile Thr Phe Ser Asn Ser Gly Met His Trp
25 30 35
Val Arg Gln Ala Pro Gly Lys Gly Leu Glu Trp Val
                        40 45
Ala Val Ile Trp Tyr Asp Gly Ser Lys Arg Tyr Tyr
        50 55 60
Ala Asp Ser Val Lys Gly Arg Phe Thr Ile Ser Arg
                                65 70
Asp Asn Ser Lys Asn Thr Leu Phe Leu Gln Met Asn
                75 80
Ser Leu Arg Ala Glu Asp Thr Ala Val Tyr Tyr Cys
85 90 95
Ala Thr Asn Asp Asp Tyr Trp Gly Gln Gly Thr Leu
                        100 105
Val Thr Val Ser Ser
        110
Light Chain Variable region of nivolumab:
(SEQ ID NO: 101)
Glu Ile Val Leu Thr Gln Ser Pro Ala Thr Leu Ser
1 5 10
Leu Ser Pro Gly Glu Arg Ala Thr Leu Ser Cys Arg
                15 20
Ala Ser Gln Ser Val Ser Ser Tyr Leu Ala Trp Tyr
25 30 35
Gln Gln Lys Pro Gly Gln Ala Pro Arg Leu Leu Ile
                        40 45
Tyr Asp Ala Ser Asn Arg Ala Thr Gly Ile Pro Ala
        50 55 60
Arg Phe Ser Gly Ser Gly Ser Gly Thr Asp Phe Thr
                                65 70
Leu Thr Ile Ser Ser Leu Glu Pro Glu Asp Phe Ala
                75 80
Val Tyr Tyr Cys Gln Gln Ser Ser Asn Trp Pro Arg
85 90 95
Thr Phe Gly Gln Gly Thr Lys Val Glu Ile Lys
                        100 105
CDR sequences of nivolumab:
HC CDR1:
(SEQ ID NO: 102)
Asn Ser Gly Met His
HC CDR2:
(SEQ ID NO: 103)
Val Ile Trp Tyr Asp Gly Ser Lys Arg Tyr Tyr Ala
Asp Ser Val Lys Gly
HC CDR3:
(SEQ ID NO: 104)
Asn Asp Asp Tyr
LC CDR1:
(SEQ ID NO: 105)
Arg Ala Ser Gln Ser Val Ser Ser Tyr Leu Ala
LC CDR2:
(SEQ ID NO: 106)
Asp Ala Ser Asn Arg Ala Thr
LC CDR3:
(SEQ ID NO: 107)
Gln Gln Ser Ser Asn Trp Pro Arg Thr
```

In another embodiment, the anti-PD-1 ABP of a combination of the invention, or a method or use thereof, comprises one or more (e.g. all) of the CDRs or VH or VL or HC (heavy chain) or LC (light chain) sequences of nivolumab, or sequences with at least 90% (e.g., 90%, 91%, 92%, 93%, 94%, 95%, 96%, 97%, 98%, or 99%, 100%) sequence identity thereto.

The HC and LC CDRs of nivolumab are provided herein. In one embodiment, the anti-PD-1 ABP of a combination of the invention, or a method or use thereof, comprises: (a) a heavy chain variable region CDR1 of nivolumab; (b) a heavy chain variable region CDR2 of nivolumab; (c) a heavy chain variable region CDR3 of nivolumab; (d) a light chain variable region CDR1 of nivolumab; (e) a light chain variable region CDR2 of nivolumab; and (f) a light chain variable region CDR3 of nivolumab.

In another embodiment, the anti-PD-1 of a combination of the invention, or a method or use thereof, comprises: a heavy chain variable region CDR1 of nivolumab; a heavy chain variable region CDR2 of nivolumab and/or a heavy chain variable region CDR3 of nivolumab.

In another embodiment, the anti-PD-1 of a combination of the invention, or a method or use thereof, comprises: a light chain variable region CDR1 of nivolumab; a light chain variable region CDR2 of nivolumab and/or a light chain variable region CDR3 of nivolumab.

In another embodiment, the anti-PD-1 ABP of a combination of the invention, or a method or use thereof, comprises: a light chain variable region (“VL”) of nivolumab, or an amino acid sequence with at least 90% (e.g., 90, 91, 92, 93, 94, 95, 96, 97, 98, or 99%) identity to the amino acid sequence of the VL of nivolumab.

In another embodiment, the anti-PD-1 ABP of a combination of the invention, or a method or use thereof, comprises a heavy chain variable region (“VH”) of nivolumab, or an amino acid sequence with at least 90% (e.g., 90, 91, 92, 93, 94, 95, 96, 97, 98, or 99%) identity to the amino acid sequence of the VH of nivolumab.

In another embodiment, the anti-PD-1 ABP of a combination of the invention, or a method or use thereof, comprises: a light chain variable region (“VL”) of nivolumab, or an amino acid sequence with at least 90% (e.g., 90, 91, 92, 93, 94, 95, 96, 97, 98, or 99%) identity to the amino acid sequence of the VL of nivolumab and the anti-PD-1 ABP of a combination of the invention, or a method or use thereof, comprises a heavy chain variable region (“VH”) of nivolumab, or an amino acid sequence with at least 90% (e.g., 90, 91, 92, 93, 94, 95, 96, 97, 98, or 99%) identity to the amino acid sequence of the VH of nivolumab.

In another embodiment, the anti-PD-1 ABP of a combination of the invention, or a method or use thereof, comprises: a light chain (“LC”) of nivolumab, or an amino acid sequence with at least 90% (e.g., 90, 91, 92, 93, 94, 95, 96, 97, 98, or 99%) identity to the amino acid sequence of the LC of nivolumab.

In another embodiment, the anti-PD-1 ABP of a combination of the invention, or a method or use thereof, comprises a heavy chain (“HC”) of nivolumab, or an amino acid sequence with at least 90% (e.g., 90, 91, 92, 93, 94, 95, 96, 97, 98, or 99%) identity to the amino acid sequence of the HC of nivolumab.

In another embodiment, the anti-PD-1 ABP of a combination of the invention, or a method or use thereof, comprises: a light chain (“LC”) of nivolumab, or an amino acid sequence with at least 90% (e.g., 90, 91, 92, 93, 94, 95, 96, 97, 98, or 99%) identity to the amino acid sequence of the LC of nivolumab and the anti-PD-1 ABP of a combination of the invention, or a method or use thereof, comprises a heavy chain (“HC”) of nivolumab, or an amino acid sequence with at least 90% (e.g., 90, 91, 92, 93, 94, 95, 96, 97, 98, or 99%) identity to the amino acid sequence of the HC of nivolumab.

Another embodiment of a combination of the invention, or a method or use thereof, includes CDRs, VH regions, and VL regions, HC, and LC, and antibodies and nucleic acids encoding the same as disclosed in the below Sequence Listing.

An anti-OX40 ABP (e.g., an agonist ABP, e.g. an anti-hOX40 ABP, e.g. antibody), e.g., an antibody described herein, can be used in combination with an ABP (e.g., antagonist ABP, e.g antagonist antibody) against PD-1 (e.g. human PD-1). For example, an anti-OX40 antibody can be used in combination with nivolumab.

In one embodiment, the present invention provides methods of treating cancer in a mammal in need thereof comprising administering a therapeutically effective amount of an antigen binding protein that binds OX40 and an antigen binding protein that binds PD-1. In some aspects the cancer is a solid tumor. The cancer is selected from the group consisting of: melanoma, lung cancer, kidney cancer, breast cancer, head and neck cancer, colon cancer, ovarian cancer, pancreatic cancer, liver cancer, prostate cancer, bladder cancer, and gastric cancer. In another aspect the cancer is a liquid tumor.

In one embodiment, the antigen binding protein that binds OX40 and the antigen binding that binds PD-1 are administered at the same time. In another embodiment, antigen binding protein that binds OX40 and the antigen binding protein that binds PD-1 are administered sequentially, in any order. In one aspect, the antigen binding protein that binds OX40 and/or the antigen binding protein that binds PD-1 are administered systemically, e.g. intravenously. In another aspect, the antigen binding protein that binds OX40 and/or the antigen binding protein that binds PD-1 is administered intratumorally.

In one embodiment, the mammal is human.

Methods are provided wherein the tumor size of the cancer in said mammal is reduced by more than an additive amount compared with treatment with the antigen binding protein to OX-40 and the antigen binding protein to PD-1 as used as monotherapy. Suitably the combination may be synergistic.

In one embodiment, the antigen binding protein that binds OX40 binds to human OX40. In one embodiment, the antigen binding protein that binds to PD-1 binds to human PD-1. In one embodiment, the antigen binding protein that binds OX40 and/or the antigen binding protein that binds PD-1 is a humanized monoclonal antibody. In one embodiment, the antigen binding protein that binds OX40 and/or the antigen binding protein that binds PD-1 is a fully human monoclonal antibody.

The antigen binding protein that binds OX40 is an antibody with an IgG1 isotype or variant thereof. In one embodiment, the antigen binding protein that binds PD-1 is an antibody with an IgG1 isotype or variant thereof. The antigen binding protein that binds OX40 is an antibody with an IgG4 isotype or variant thereof. In one embodiment, the antigen binding protein that binds PD-1 is an antibody with an IgG4 isotype or variant thereof. In one aspect the antigen binding protein that binds OX40 is an agonist antibody. In one aspect the antigen binding protein that binds PD-1 is an antagonist antibody.

Suitably, the antigen binding protein that binds OX40 comprises: a heavy chain variable region CDR1 comprising an amino acid sequence with at least 90% 91%, 92%, 93%, 94%, 95%, 96%, 97%, 98%, 99% or 100% sequence identity to the amino acid sequence set forth in SEQ ID NO: 1 or 13; a heavy chain variable region CDR2 comprising an amino acid sequence with at least 90%, 91%, 92%, 93%, 94%, 95%, 96%, 97%, 98%, 99% or 100% sequence identity to the amino acid sequence as set forth in SEQ ID NO: 2 or 14; and/or a heavy chain variable region CDR3 comprising an amino acid sequence with at least 90%, 91%, 92%, 93%, 94%, 95%, 96%, 97%, 98%, 99% or 100% sequence identity to the amino acid sequence as set forth in SEQ ID NO: 3 or 15.

Suitably, the antigen binding protein that binds OX40 comprises a light chain variable region CDR1 comprising an amino acid sequence with at least 90%, 91%, 92%, 93%, 94%, 95%, 96%, 97%, 98%, 99% or 100% sequence identity to the amino acid sequence as set forth in SEQ ID NO: 7 or 19; a light chain variable region CDR2 comprising an amino acid sequence with at least at least 90%, 91%, 92%, 93%, 94%, 95%, 96%, 97%, 98%, 99% or 100% sequence identity to the amino acid sequence as set forth in SEQ ID NO: 8 or 20 and/or a light chain variable region CDR3 comprising an amino acid sequence with at least 90%, 91%, 92%, 93%, 94%, 95%, 96%, 97%, 98%, 99% or 100% sequence identity to the amino acid sequence as set forth in SEQ ID NO: 9 or 21.

Suitably, the antigen binding protein that binds OX40 comprises: (a) a heavy chain variable region CDR1 comprising the amino acid sequence of SEQ ID NO: 1; (b) a heavy chain variable region CDR2 comprising the amino acid sequence of SEQ ID NO: 2; (c) a heavy chain variable region CDR3 comprising the amino acid sequence of SEQ ID NO. 3; (d) a light chain variable region CDR1 comprising the amino acid sequence of SEQ ID NO. 7; (e) a light chain variable region CDR2 comprising the amino acid sequence of SEQ ID NO. 8; and (f) a light chain variable region CDR3 comprising the amino acid sequence of SEQ ID NO. 9.

Suitably, the antigen binding protein that binds OX40 comprises: (a) a heavy chain variable region CDR1 comprising the amino acid sequence of SEQ ID NO: 13; (b) a heavy chain variable region CDR2 comprising the amino acid sequence of SEQ ID NO: 14; (c) a heavy chain variable region CDR3 comprising the amino acid sequence of SEQ ID NO. 15; (d) a light chain variable region CDR1 comprising the amino acid sequence of SEQ ID NO. 19; (e) a light chain variable region CDR2 comprising the amino acid sequence of SEQ ID NO. 20; and (f) a light chain variable region CDR3 comprising the amino acid sequence of SEQ ID NO. 21.

Suitably, the antigen binding protein that binds OX40 comprises a light chain variable region (“VL”) comprising an amino acid sequence with at least 90%, 91%, 92%, 93%, 94%, 95%, 96%, 97%, 98%, 99% or 100% sequence identity to the amino acid sequence as set forth in SEQ ID NO: 10, 11, 22 or 23. Suitably, the antigen binding protein that binds OX40 comprises a heavy chain variable region (“VH”) comprising an amino acid sequence with at least 90%, 91%, 92%, 93%, 94%, 95%, 96%, 97%, 98%, 99% or 100% sequence identity to the amino acid sequence as set forth in SEQ ID NO: 4, 5, 16 and 17. Suitably, the antigen binding protein that binds OX40 comprises a heavy chain variable region comprising the amino acid sequence set forth in SEQ ID NO:5 and a light chain variable region comprising the amino acid sequence set forth in SEQ ID NO: 11.

Suitably, the antigen binding protein that binds OX40 comprises a heavy chain variable region comprising the amino acid sequence set forth in SEQ ID NO:17 and a light chain variable region comprising the amino acid sequence set forth in SEQ ID NO: 23. Suitably, the antigen binding protein that binds OX40 comprises a light chain variable region comprising the amino acid sequence of SEQ ID NO: 11 or 23, or an amino acid sequence with at least 90% sequence identity to the amino acid sequences of SEQ ID NO: 11 or 23. Suitably, the antigen binding protein that binds OX40 comprises a heavy chain variable region comprising the amino acid sequence of SEQ ID NO: 5 or 17, or an amino acid sequence with at least 90% sequence identity to the amino acid sequences of SEQ ID NO: 5 or 17.

In one embodiment, the antigen binding protein that binds PD-1 is pembrolizumab, or an antibody comprising 90%, 91%, 92%, 93%, 94%, 95%, 96%, 97%, 98%, 99% or 100% sequence identity thereto. In another embodiment, the antigen binding protein that binds PD-1 is nivolumab, or an antibody having 90%, 91%, 92%, 93%, 94%, 95%, 96%, 97%, 98%, 99% or 100% sequence identity thereto.

In one aspect, the mammal has increased survival when treated with a therapeutically effective amount of an antigen binding protein to OX-40 and therapeutically effective amount of an antigen binding protein to PD-1 compared with a mammal who received the antigen binding protein to OX-40 or the antigen binding protein to PD-1 as monotherapy. In one aspect, the methods further comprise administering at least one anti-neoplastic agent to the mammal in need thereof.

In one embodiment, pharmaceutical compositions are provided comprising a therapeutically effective amount of an antigen binding protein that binds OX40 and a therapeutically effective amount of an antigen binding protein that binds PD-1.

In one embodiment, the pharmaceutical compositions comprise an antibody comprising an antigen binding protein that binds OX40 comprising a CDRH1 having an amino acid sequence with at least 90%, 91%, 92%, 93%, 94%, 95%, 96%, 97%, 98%, 99% or 100% sequence identity to the amino acid sequence as set forth in SEQ ID NO: 1, a CDRH2 having an amino acid sequence with at least 90%, 91%, 92%, 93%, 94%, 95%, 96%, 97%, 98%, 99% or 100% sequence identity to the amino acid sequence as set forth in SEQ ID NO: 2, a CDRH3 having an amino acid sequence with at least 90%, 91%, 92%, 93%, 94%, 95%, 96%, 97%, 98%, 99% or 100% sequence identity to the amino acid sequence as set forth in SEQ ID NO: 3, a CDRL1 having an amino acid sequence with at least 90%, 91%, 92%, 93%, 94%, 95%, 96%, 97%, 98%, 99% or 100% sequence identity to the amino acid sequence as set forth in SEQ ID NO: 7, a CDRL2 having an amino acid sequence with at least 90%, 91%, 92%, 93%, 94%, 95%, 96%, 97%, 98%, 99% or 100% sequence identity to the amino acid sequence as set forth in SEQ ID NO: 8, a CDRL3 having an amino acid sequence with at least 90%, 91%, 92%, 93%, 94%, 95%, 96%, 97%, 98%, 99% or 100% sequence identity to the amino acid sequence as set forth in SEQ ID NO: 9; and pembrolizumab, or an antibody comprising 90%, 91%, 92%, 93%, 94%, 95%, 96%, 97%, 98%, 99% or 100% sequence identity thereto.

In one embodiment, the pharmaceutical compositions of the present invention comprise an antibody comprising a VH region having a sequence at least with a sequence at least 90%, 91%, 92%, 93%, 94%, 95%, 96%, 97%, 98%, 99% or 100% sequence identity to the amino acid sequence as set forth in SEQ ID NO: 4 or 5 and VL having a sequence at least with a sequence at least 90%, 91%, 92%, 93%, 94%, 95%, 96%, 97%, 98%, 99% or 100% sequence identity to the amino acid sequence as set forth in SEQ ID NO:10 or 11, and pembrolizumab, or an antibody comprising 90%, 91%, 92%, 93%, 94%, 95%, 96%, 97%, 98%, 99% or 100% sequence identity thereto.

In one embodiment, the pharmaceutical compositions of the present invention comprise an antibody comprising an antigen binding protein that binds OX40 comprising a CDRH1 having an amino acid sequence with at least 90%, 91%, 92%, 93%, 94%, 95%, 96%, 97%, 98%, 99% or 100% sequence identity to the amino acid sequence as set forth in SEQ ID NO: 1, a CDRH2 having an amino acid sequence with at least 90%, 91%, 92%, 93%, 94%, 95%, 96%, 97%, 98%, 99% or 100% sequence identity to the amino acid sequence as set forth in SEQ ID NO: 2, a CDRH3 having an amino acid sequence with at least 90%, 91%, 92%, 93%, 94%, 95%, 96%, 97%, 98%, 99% or 100% sequence identity to the amino acid sequence as set forth in SEQ ID NO: 3, a CDRL1 having an amino acid sequence with at least 90%, 91%, 92%, 93%, 94%, 95%, 96%, 97%, 98%, 99% or 100% sequence identity to the amino acid sequence as set forth in SEQ ID NO: 7, a CDRL2 having an amino acid sequence with at least 90%, 91%, 92%, 93%, 94%, 95%, 96%, 97%, 98%, 99% or 100% sequence identity to the amino acid sequence as set forth in SEQ ID NO: 8, a CDRL3 having an amino acid sequence with at least 90%, 91%, 92%, 93%, 94%, 95%, 96%, 97%, 98%, 99% or 100% sequence identity to the amino acid sequence as set forth in SEQ ID NO: 9; and nivolumab, or an antibody comprising 90%, 91%, 92%, 93%, 94%, 95%, 96%, 97%, 98%, 99% or 100% sequence identity thereto.

Also provided in the present invention are the use of a combination or pharmaceutical compositions of this invention in the manufacture of a medicament for the treatment of cancer. Also provided are the use of pharmaceutical compositions of the present invention for treating cancer. The present invention also provides combination kit comprising pharmaceutical compositions of the invention together with one or more pharmaceutically acceptable carriers.

In one embodiment methods are provided for reducing tumor size in a human having cancer comprising administering a therapeutically effective amount of an agonist antibody to human OX-40 and a therapeutically effective amount of an antagonist antibody to human PD-1.

### Methods of Treatment

The combinations of the invention are believed to have utility in disorders wherein the engagement of OX40 (e.g., agonistic engagement) and/or PD-1 (e.g., antagonistic engagement), is beneficial.

The present invention thus also provides a combination of the invention, for use in therapy, particularly in the treatment of disorders wherein the engagement of OX40 (e.g., agonistic engagement) and/or PD-1 (e.g., antagonistic engagement), is beneficial, particularly cancer.

A further aspect of the invention provides a method of treatment of a disorder wherein engagement of OX40 (e.g., agonistic engagement) and/or PD-1 (e.g., antagonistic engagement), comprising administering a combination of the invention.

A further aspect of the present invention provides the use of a combination of the invention in the manufacture of a medicament for the treatment of a disorder engagement of OX40 (e.g., agonistic engagement) and/or PD-1 (e.g., antagonistic engagement), is beneficial. In preferred embodiments the disorder is cancer.

Examples of cancers that are suitable for treatment with combination of the invention include, but are limited to, both primary and metastatic forms of head and neck, breast, lung, colon, ovary, and prostate cancers. Suitably the cancer is selected from: brain (gliomas), glioblastomas, astrocytomas, glioblastoma multiforme, Bannayan-Zonana syndrome, Cowden disease, Lhermitte-Duclos disease, breast, inflammatory breast cancer, Wilm's tumor, Ewing's sarcoma, Rhabdomyosarcoma, ependymoma, medulloblastoma, colon, head and neck, kidney, lung, liver, melanoma, ovarian, pancreatic, prostate, sarcoma, osteosarcoma, giant cell tumor of bone, thyroid, lymphoblastic T cell leukemia, Chronic myelogenous leukemia, Chronic lymphocytic leukemia, Hairy-cell leukemia, acute lymphoblastic leukemia, acute myelogenous leukemia, AML, Chronic neutrophilic leukemia, Acute lymphoblastic T cell leukemia, plasmacytoma, Immunoblastic large cell leukemia, Mantle cell leukemia, Multiple myeloma Megakaryoblastic leukemia, multiple myeloma, acute megakaryocytic leukemia, promyelocytic leukemia, Erythroleukemia, malignant lymphoma, hodgkins lymphoma, non-hodgkins lymphoma, lymphoblastic T cell lymphoma, Burkitt's lymphoma, follicular lymphoma, neuroblastoma, bladder cancer, urothelial cancer, lung cancer, vulval cancer, cervical cancer, endometrial cancer, renal cancer, mesothelioma, esophageal cancer, salivary gland cancer, hepatocellular cancer, gastric cancer, nasopharangeal cancer, buccal cancer, cancer of the mouth, GIST (gastrointestinal stromal tumor) and testicular cancer.

Additionally, examples of a cancer to be treated include Barret's adenocarcinoma; billiary tract carcinomas; breast cancer; cervical cancer; cholangiocarcinoma; central nervous system tumors including primary CNS tumors such as glioblastomas, astrocytomas (e.g., glioblastoma multiforme) and ependymomas, and secondary CNS tumors (i.e., metastases to the central nervous system of tumors originating outside of the central nervous system); colorectal cancer including large intestinal colon carcinoma; gastric cancer; carcinoma of the head and neck including squamous cell carcinoma of the head and neck; hematologic cancers including leukemias and lymphomas such as acute lymphoblastic leukemia, acute myelogenous leukemia (AML), myelodysplastic syndromes, chronic myelogenous leukemia, Hodgkin's lymphoma, non-Hodgkin's lymphoma, megakaryoblastic leukemia, multiple myeloma and erythroleukemia; hepatocellular carcinoma; lung cancer including small cell lung cancer and non-small cell lung cancer; ovarian cancer; endometrial cancer; pancreatic cancer; pituitary adenoma; prostate cancer; renal cancer; sarcoma; skin cancers including melanomas; and thyroid cancers.

Suitably, the present invention relates to a method for treating or lessening the severity of a cancer selected from: brain (gliomas), glioblastomas, astrocytomas, glioblastoma multiforme, Bannayan-Zonana syndrome, Cowden disease, Lhermitte-Duclos disease, breast, colon, head and neck, kidney, lung, liver, melanoma, ovarian, pancreatic, prostate, sarcoma and thyroid.

Suitably, the present invention relates to a method for treating or lessening the severity of a cancer selected from ovarian, breast, pancreatic and prostate.

Suitably, the present invention relates to a method for treating or lessening the severity of NSCLC (non small cell lung cancer), squamous cell carcinoma of the head and neck (SCCHN), renal cell carcinoma (RCC), melanoma, bladder cancer, soft tissue sarcoma (STS), triple-negative breast cancer (TNBC), and colorectal carcinoma displaying microsatellite instability (MSI CRC).

Suitably, the present invention relates to a method for treating or lessening the severity of melanoma, e.g. metastatic melanoma.

Suitably, the present invention relates to a method for treating or lessening the severity of squamous non-small cell lung cancer, e.g. metastatic squamous non-small cell lung cancer.

Suitably the present invention relates to a method for treating or lessening the severity of pre-cancerous syndromes in a mammal, including a human, wherein the pre-cancerous syndrome is selected from: cervical intraepithelial neoplasia, monoclonal gammapathy of unknown significance (MGUS), myelodysplastic syndrome, aplastic anemia, cervical lesions, skin nevi (pre-melanoma), prostatic intraepithleial (intraductal) neoplasia (PIN), Ductal Carcinoma in situ (DCIS), colon polyps and severe hepatitis or cirrhosis.

The combination of the invention may be used alone or in combination with one or more other therapeutic agents. The invention thus provides in a further aspect a further combination comprising a combination of the invention with a further therapeutic agent or agents, compositions and medicaments comprising the combination and use of the further combination, compositions and medicaments in therapy, in particular in the treatment of diseases susceptible engagement of OX40, e.g. agonism of OX40, and/or PD-1, e.g. antagonism of PD-1.

In the embodiment, the combination of the invention may be employed with other therapeutic methods of cancer treatment. In particular, in anti-neoplastic therapy, combination therapy with other chemotherapeutic, hormonal, antibody agents as well as surgical and/or radiation treatments other than those mentioned above are envisaged. Combination therapies according to the present invention thus include the administration of an anti-OX40 ABP of a combination, or method or use thereof, of the invention and/or an anti-PD-1 ABP of a combination, or method or use thereof, of the invention as well as optional use of other therapeutic agents including other anti-neoplastic agents. Such combination of agents may be administered together or separately and, when administered separately this may occur simultaneously or sequentially in any order, both close and remote in time. In one embodiment, the pharmaceutical combination includes an anti-OX40 ABP, suitably an agonist anti-OX40 ABP and an anti-PD-1 ABP, suitably an antagonist anti-PD1 ABP, and optionally at least one additional anti-neoplastic agent.

In one embodiment, the further anti-cancer therapy is surgical and/or radiotherapy.

In one embodiment, the further anti-cancer therapy is at least one additional anti-neoplastic agent.

Any anti-neoplastic agent that has activity versus a susceptible tumor being treated may be utilized in the combination. Typical anti-neoplastic agents useful include, but are not limited to, anti-microtubule agents such as diterpenoids and vinca alkaloids; platinum coordination complexes; alkylating agents such as nitrogen mustards, oxazaphosphorines, alkylsulfonates, nitrosoureas, and triazenes; antibiotic agents such as anthracyclins, actinomycins and bleomycins; topoisomerase II inhibitors such as epipodophyllotoxins; antimetabolites such as purine and pyrimidine analogues and anti-folate compounds; topoisomerase I inhibitors such as camptothecins; hormones and hormonal analogues; signal transduction pathway inhibitors; non-receptor tyrosine angiogenesis inhibitors; immunotherapeutic agents; proapoptotic agents; and cell cycle signaling inhibitors.

Anti-microtubule or anti-mitotic agents: Anti-microtubule or anti-mitotic agents are phase specific agents active against the microtubules of tumor cells during M or the mitosis phase of the cell cycle. Examples of anti-microtubule agents include, but are not limited to, diterpenoids and vinca alkaloids.

Diterpenoids, which are derived from natural sources, are phase specific anti-cancer agents that operate at the G₂/M phases of the cell cycle. It is believed that the diterpenoids stabilize the β-tubulin subunit of the microtubules, by binding with this protein. Disassembly of the protein appears then to be inhibited with mitosis being arrested and cell death following. Examples of diterpenoids include, but are not limited to, paclitaxel and its analog docetaxel.

Paclitaxel, 5β,20-epoxy-1,2α,4,7β,10β,13α-hexa-hydroxytax-11-en-9-one 4,10-diacetate 2-benzoate 13-ester with (2R,3S)—N-benzoyl-3-phenylisoserine; is a natural diterpene product isolated from the Pacific yew tree Taxus brevifolia and is commercially available as an injectable solution TAXOL®. It is a member of the taxane family of terpenes. Paclitaxel has been approved for clinical use in the treatment of refractory ovarian cancer in the United States (Markman et al., Yale Journal of Biology and Medicine, 64:583, 1991; McGuire et al., Ann. Intern, Med., 111:273, 1989) and for the treatment of breast cancer (Holmes et al., J. Nat. Cancer Inst., 83:1797, 1991.) It is a potential candidate for treatment of neoplasms in the skin (Einzig et. al., Proc. Am. Soc. Clin. Oncol., 20:46) and head and neck carcinomas (Forastire et. al., Sem. Oncol., 20:56, 1990). The compound also shows potential for the treatment of polycystic kidney disease (Woo et. al., Nature, 368:750. 1994), lung cancer and malaria. Treatment of patients with paclitaxel results in bone marrow suppression (multiple cell lineages, Ignoff, R. J. et. al, Cancer Chemotherapy Pocket Guide, 1998) related to the duration of dosing above a threshold concentration (50 nM) (Kearns, C. M. et. al., Seminars in Oncology, 3(6) p. 16-23, 1995).

Docetaxel, (2R,3S)—N-carboxy-3-phenylisoserine,N-tert-butyl ester, 13-ester with 5β-20-epoxy-1,2α,4,7β,10β,13α-hexahydroxytax-11-en-9-one 4-acetate 2-benzoate, trihydrate; is commercially available as an injectable solution as TAXOTERE®. Docetaxel is indicated for the treatment of breast cancer. Docetaxel is a semisynthetic derivative of paclitaxel q.v., prepared using a natural precursor, 10-deacetyl-baccatin III, extracted from the needle of the European Yew tree.

Vinca alkaloids are phase specific anti-neoplastic agents derived from the periwinkle plant. Vinca alkaloids act at the M phase (mitosis) of the cell cycle by binding specifically to tubulin. Consequently, the bound tubulin molecule is unable to polymerize into microtubules. Mitosis is believed to be arrested in metaphase with cell death following. Examples of vinca alkaloids include, but are not limited to, vinblastine, vincristine, and vinorelbine.

Vinblastine, vincaleukoblastine sulfate, is commercially available as VELBAN® as an injectable solution. Although, it has possible indication as a second line therapy of various solid tumors, it is primarily indicated in the treatment of testicular cancer and various lymphomas including Hodgkin's Disease; and lymphocytic and histiocytic lymphomas. Myelosuppression is the dose limiting side effect of vinblastine.

Vincristine, vincaleukoblastine, 22-oxo-, sulfate, is commercially available as ONCOVIN® as an injectable solution. Vincristine is indicated for the treatment of acute leukemias and has also found use in treatment regimens for Hodgkin's and non-Hodgkin's malignant lymphomas. Alopecia and neurologic effects are the most common side effect of vincristine and to a lesser extent myelosupression and gastrointestinal mucositis effects occur.

Vinorelbine, 3′,4′-didehydro-4′-deoxy-C′-norvincaleukoblastine [R—(R*,R*)-2,3-dihydroxybutanedioate (1:2)(salt)], commercially available as an injectable solution of vinorelbine tartrate (NAVELBINE®), is a semisynthetic vinca alkaloid. Vinorelbine is indicated as a single agent or in combination with other chemotherapeutic agents, such as cisplatin, in the treatment of various solid tumors, particularly non-small cell lung, advanced breast, and hormone refractory prostate cancers. Myelosuppression is the most common dose limiting side effect of vinorelbine.

Platinum coordination complexes: Platinum coordination complexes are non-phase specific anti-cancer agents, which are interactive with DNA. The platinum complexes enter tumor cells, undergo, aquation and form intra- and interstrand crosslinks with DNA causing adverse biological effects to the tumor. Examples of platinum coordination complexes include, but are not limited to, oxaliplatin, cisplatin and carboplatin.

Cisplatin, cis-diamminedichloroplatinum, is commercially available as PLATINOL® as an injectable solution. Cisplatin is primarily indicated in the treatment of metastatic testicular and ovarian cancer and advanced bladder cancer.

Carboplatin, platinum, diammine [1,1-cyclobutane-dicarboxylate(2-)—O,O′], is commercially available as PARAPLATIN® as an injectable solution. Carboplatin is primarily indicated in the first and second line treatment of advanced ovarian carcinoma.

Alkylating agents: Alkylating agents are non-phase anti-cancer specific agents and strong electrophiles. Typically, alkylating agents form covalent linkages, by alkylation, to DNA through nucleophilic moieties of the DNA molecule such as phosphate, amino, sulfhydryl, hydroxyl, carboxyl, and imidazole groups. Such alkylation disrupts nucleic acid function leading to cell death. Examples of alkylating agents include, but are not limited to, nitrogen mustards such as cyclophosphamide, melphalan, and chlorambucil; alkyl sulfonates such as busulfan; nitrosoureas such as carmustine; and triazenes such as dacarbazine.

Cyclophosphamide, 2-[bis(2-chloroethyl)amino]tetrahydro-2H-1,3,2-oxazaphosphorine 2-oxide monohydrate, is commercially available as an injectable solution or tablets as CYTOXAN®. Cyclophosphamide is indicated as a single agent or in combination with other chemotherapeutic agents, in the treatment of malignant lymphomas, multiple myeloma, and leukemias.

Melphalan, 4-[bis(2-chloroethyl)amino]-L-phenylalanine, is commercially available as an injectable solution or tablets as ALKERAN®. Melphalan is indicated for the palliative treatment of multiple myeloma and non-resectable epithelial carcinoma of the ovary. Bone marrow suppression is the most common dose limiting side effect of melphalan.

Chlorambucil, 4-[bis(2-chloroethyl)amino]benzenebutanoic acid, is commercially available as LEUKERAN® tablets. Chlorambucil is indicated for the palliative treatment of chronic lymphatic leukemia, and malignant lymphomas such as lymphosarcoma, giant follicular lymphoma, and Hodgkin's disease.

Busulfan, 1,4-butanediol dimethanesulfonate, is commercially available as MYLERAN® TABLETS. Busulfan is indicated for the palliative treatment of chronic myelogenous leukemia.

Carmustine, 1,3-[bis(2-chloroethyl)-1-nitrosourea, is commercially available as single vials of lyophilized material as BiCNU®. Carmustine is indicated for the palliative treatment as a single agent or in combination with other agents for brain tumors, multiple myeloma, Hodgkin's disease, and non-Hodgkin's lymphomas.

Dacarbazine, 5-(3,3-dimethyl-1-triazeno)-imidazole-4-carboxamide, is commercially available as single vials of material as DTIC-Dome®. Dacarbazine is indicated for the treatment of metastatic malignant melanoma and in combination with other agents for the second line treatment of Hodgkin's Disease.

Antibiotic anti-neoplastics: Antibiotic anti-neoplastics are non-phase specific agents, which bind or intercalate with DNA. Typically, such action results in stable DNA complexes or strand breakage, which disrupts ordinary function of the nucleic acids leading to cell death. Examples of antibiotic anti-neoplastic agents include, but are not limited to, actinomycins such as dactinomycin, anthrocyclins such as daunorubicin and doxorubicin; and bleomycins.

Dactinomycin, also know as Actinomycin D, is commercially available in injectable form as COSMEGEN®. Dactinomycin is indicated for the treatment of Wilm's tumor and rhabdomyosarcoma.

Daunorubicin, (8S-cis-)-8-acetyl-10-[(3-amino-2,3,6-trideoxy-α-L-lyxo-hexopyranosyl)oxy]-7,8,9,10-tetrahydro-6,8,11-trihydroxy-1-methoxy-5,12 naphthacenedione hydrochloride, is commercially available as a liposomal injectable form as DAUNOXOME® or as an injectable as CERUBIDINE®. Daunorubicin is indicated for remission induction in the treatment of acute nonlymphocytic leukemia and advanced HIV associated Kaposi's sarcoma.

Doxorubicin, (8S, 10S)-10-[(3-amino-2,3,6-trideoxy-α-L-lyxo-hexopyranosyl)oxy]-8-glycoloyl, 7,8,9,10-tetrahydro-6,8,11-trihydroxy-1-methoxy-5,12 naphthacenedione hydrochloride, is commercially available as an injectable form as RUBEX® or ADRIAMYCIN RDF®. Doxorubicin is primarily indicated for the treatment of acute lymphoblastic leukemia and acute myeloblastic leukemia, but is also a useful component in the treatment of some solid tumors and lymphomas.

Bleomycin, a mixture of cytotoxic glycopeptide antibiotics isolated from a strain of Streptomyces verticillus, is commercially available as BLENOXANE®. Bleomycin is indicated as a palliative treatment, as a single agent or in combination with other agents, of squamous cell carcinoma, lymphomas, and testicular carcinomas.

Topoisomerase II inhibitors: Topoisomerase II inhibitors include, but are not limited to, epipodophyllotoxins.

Epipodophyllotoxins are phase specific anti-neoplastic agents derived from the mandrake plant. Epipodophyllotoxins typically affect cells in the S and G₂ phases of the cell cycle by forming a ternary complex with topoisomerase II and DNA causing DNA strand breaks. The strand breaks accumulate and cell death follows. Examples of epipodophyllotoxins include, but are not limited to, etoposide and teniposide.

Etoposide, 4′-demethyl-epipodophyllotoxin 9[4,6-β-(R)-ethylidene-β-D-glucopyranoside], is commercially available as an injectable solution or capsules as VePESID® and is commonly known as VP-16. Etoposide is indicated as a single agent or in combination with other chemotherapy agents in the treatment of testicular and non-small cell lung cancers.

Teniposide, 4′-demethyl-epipodophyllotoxin 9[4,6-β-(R)-thenylidene-β-D-glucopyranoside], is commercially available as an injectable solution as VUMON® and is commonly known as VM-26. Teniposide is indicated as a single agent or in combination with other chemotherapy agents in the treatment of acute leukemia in children.

Antimetabolite neoplastic agents: Antimetabolite neoplastic agents are phase specific anti-neoplastic agents that act at S phase (DNA synthesis) of the cell cycle by inhibiting DNA synthesis or by inhibiting purine or pyrimidine base synthesis and thereby limiting DNA synthesis. Consequently, S phase does not proceed and cell death follows. Examples of antimetabolite anti-neoplastic agents include, but are not limited to, fluorouracil, methotrexate, cytarabine, mecaptopurine, thioguanine, and gemcitabine.

5-fluorouracil, 5-fluoro-2,4-(1H,3H) pyrimidinedione, is commercially available as fluorouracil. Administration of 5-fluorouracil leads to inhibition of thymidylate synthesis and is also incorporated into both RNA and DNA. The result typically is cell death. 5-fluorouracil is indicated as a single agent or in combination with other chemotherapy agents in the treatment of carcinomas of the breast, colon, rectum, stomach and pancreas. Other fluoropyrimidine analogs include 5-fluoro deoxyuridine (floxuridine) and 5-fluorodeoxyuridine monophosphate.

Cytarabine, 4-amino-1-β-D-arabinofuranosyl-2 (1H)-pyrimidinone, is commercially available as CYTOSAR-U® and is commonly known as Ara-C. It is believed that cytarabine exhibits cell phase specificity at S-phase by inhibiting DNA chain elongation by terminal incorporation of cytarabine into the growing DNA chain. Cytarabine is indicated as a single agent or in combination with other chemotherapy agents in the treatment of acute leukemia. Other cytidine analogs include 5-azacytidine and 2′,2′-difluorodeoxycytidine (gemcitabine).

Mercaptopurine, 1,7-dihydro-6H-purine-6-thione monohydrate, is commercially available as PURINETHOL®. Mercaptopurine exhibits cell phase specificity at S-phase by inhibiting DNA synthesis by an as of yet unspecified mechanism. Mercaptopurine is indicated as a single agent or in combination with other chemotherapy agents in the treatment of acute leukemia. A useful mercaptopurine analog is azathioprine.

Thioguanine, 2-amino-1,7-dihydro-6H-purine-6-thione, is commercially available as TABLOID®. Thioguanine exhibits cell phase specificity at S-phase by inhibiting DNA synthesis by an as of yet unspecified mechanism. Thioguanine is indicated as a single agent or in combination with other chemotherapy agents in the treatment of acute leukemia. Other purine analogs include pentostatin, erythrohydroxynonyladenine, fludarabine phosphate, and cladribine.

Gemcitabine, 2′-deoxy-2′, 2′-difluorocytidine monohydrochloride (β-isomer), is commercially available as GEMZAR®. Gemcitabine exhibits cell phase specificity at S-phase and by blocking progression of cells through the G1/S boundary. Gemcitabine is indicated in combination with cisplatin in the treatment of locally advanced non-small cell lung cancer and alone in the treatment of locally advanced pancreatic cancer.

Methotrexate, N-[4[[(2,4-diamino-6-pteridinyl) methyl]methylamino]benzoyl]-L-glutamic acid, is commercially available as methotrexate sodium. Methotrexate exhibits cell phase effects specifically at S-phase by inhibiting DNA synthesis, repair and/or replication through the inhibition of dyhydrofolic acid reductase which is required for synthesis of purine nucleotides and thymidylate. Methotrexate is indicated as a single agent or in combination with other chemotherapy agents in the treatment of choriocarcinoma, meningeal leukemia, non-Hodgkin's lymphoma, and carcinomas of the breast, head, neck, ovary and bladder.

Topoisomerase I inhibitors: Camptothecins, including, camptothecin and camptothecin derivatives are available or under development as Topoisomerase I inhibitors. Camptothecins cytotoxic activity is believed to be related to its Topoisomerase I inhibitory activity. Examples of camptothecins include, but are not limited to irinotecan, topotecan, and the various optical forms of 7-(4-methylpiperazino-methylene)-10,11-ethylenedioxy-20-camptothecin described below.

Irinotecan HCl, (45)-4,11-diethyl-4-hydroxy-9-[(4-piperidinopiperidino) carbonyloxy]-1H-pyrano[3′,4′,6,7]indolizino[1,2-b]quinoline-3,14(4H,12H)-dione hydrochloride, is commercially available as the injectable solution CAMPTOSAR®. Irinotecan is a derivative of camptothecin which binds, along with its active metabolite SN-38, to the topoisomerase I-DNA complex. It is believed that cytotoxicity occurs as a result of irreparable double strand breaks caused by interaction of the topoisomerase I:DNA:irintecan or SN-38 ternary complex with replication enzymes. Irinotecan is indicated for treatment of metastatic cancer of the colon or rectum.

Topotecan HCl, (5)-10-[(dimethylamino)methyl]-4-ethyl-4,9-dihydroxy-1H-pyrano[3′,4′,6,7]indolizino[1,2-b]quinoline-3,14-(4H,12H)-dione monohydrochloride, is commercially available as the injectable solution HYCAMTIN®. Topotecan is a derivative of camptothecin which binds to the topoisomerase I-DNA complex and prevents religation of singles strand breaks caused by Topoisomerase I in response to torsional strain of the DNA molecule. Topotecan is indicated for second line treatment of metastatic carcinoma of the ovary and small cell lung cancer.

Hormones and hormonal analogues: Hormones and hormonal analogues are useful compounds for treating cancers in which there is a relationship between the hormone(s) and growth and/or lack of growth of the cancer. Examples of hormones and hormonal analogues useful in cancer treatment include, but are not limited to, adrenocorticosteroids such as prednisone and prednisolone which are useful in the treatment of malignant lymphoma and acute leukemia in children; aminoglutethimide and other aromatase inhibitors such as anastrozole, letrazole, vorazole, and exemestane useful in the treatment of adrenocortical carcinoma and hormone dependent breast carcinoma containing estrogen receptors; progestrins such as megestrol acetate useful in the treatment of hormone dependent breast cancer and endometrial carcinoma; estrogens, androgens, and anti-androgens such as flutamide, nilutamide, bicalutamide, cyproterone acetate and 5α-reductases such as finasteride and dutasteride, useful in the treatment of prostatic carcinoma and benign prostatic hypertrophy; anti-estrogens such as tamoxifen, toremifene, raloxifene, droloxifene, iodoxyfene, as well as selective estrogen receptor modulators (SERMS) such those described in U.S. Pat. Nos. 5,681,835, 5,877,219, and 6,207,716, useful in the treatment of hormone dependent breast carcinoma and other susceptible cancers; and gonadotropin-releasing hormone (GnRH) and analogues thereof which stimulate the release of leutinizing hormone (LH) and/or follicle stimulating hormone (FSH) for the treatment prostatic carcinoma, for instance, LHRH agonists and antagagonists such as goserelin acetate and luprolide.

Signal transduction pathway inhibitors: Signal transduction pathway inhibitors are those inhibitors, which block or inhibit a chemical process which evokes an intracellular change. As used herein this change is cell proliferation or differentiation. Signal transduction inhibitors useful in the present invention include inhibitors of receptor tyrosine kinases, non-receptor tyrosine kinases, SH2/SH3 domain blockers, serine/threonine kinases, phosphotidyl inositol-3 kinases, myo-inositol signaling, and Ras oncogenes.

Several protein tyrosine kinases catalyse the phosphorylation of specific tyrosyl residues in various proteins involved in the regulation of cell growth. Such protein tyrosine kinases can be broadly classified as receptor or non-receptor kinases.

Receptor tyrosine kinases are transmembrane proteins having an extracellular ligand binding domain, a transmembrane domain, and a tyrosine kinase domain. Receptor tyrosine kinases are involved in the regulation of cell growth and are generally termed growth factor receptors. Inappropriate or uncontrolled activation of many of these kinases, i.e. aberrant kinase growth factor receptor activity, for example by over-expression or mutation, has been shown to result in uncontrolled cell growth. Accordingly, the aberrant activity of such kinases has been linked to malignant tissue growth. Consequently, inhibitors of such kinases could provide cancer treatment methods. Growth factor receptors include, for example, epidermal growth factor receptor (EGFr), platelet derived growth factor receptor (PDGFr), erbB2, erbB4, ret, vascular endothelial growth factor receptor (VEGFr), tyrosine kinase with immunoglobulin-like and epidermal growth factor identity domains (TIE-2), insulin growth factor-I (IGFI) receptor, macrophage colony stimulating factor (cfms), BTK, ckit, cmet, fibroblast growth factor (FGF) receptors, Trk receptors (TrkA, TrkB, and TrkC), ephrin (eph) receptors, and the RET protooncogene. Several inhibitors of growth receptors are under development and include ligand antagonists, antibodies, tyrosine kinase inhibitors and anti-sense oligonucleotides. Growth factor receptors and agents that inhibit growth factor receptor function are described, for instance, in Kath, John C., Exp. Opin. Ther. Patents (2000) 10(6):803-818; Shawver et al DDT Vol 2, No. 2 Feb. 1997; and Lofts, F. J. et al, “Growth factor receptors as targets”, New Molecular Targets for Cancer Chemotherapy, ed. Workman, Paul and Kerr, David, CRC press 1994, London.

Tyrosine kinases, which are not growth factor receptor kinases are termed non-receptor tyrosine kinases. Non-receptor tyrosine kinases useful in the present invention, which are targets or potential targets of anti-cancer drugs, include cSrc, Lck, Fyn, Yes, Jak, cAbl, FAK (Focal adhesion kinase), Brutons tyrosine kinase, and Bcr-Abl. Such non-receptor kinases and agents which inhibit non-receptor tyrosine kinase function are described in Sinh, S. and Corey, S. J., (1999) Journal of Hematotherapy and Stem Cell Research 8 (5): 465-80; and Bolen, J. B., Brugge, J. S., (1997) Annual review of Immunology. 15: 371-404.

SH2/SH3 domain blockers are agents that disrupt SH2 or SH3 domain binding in a variety of enzymes or adaptor proteins including, PI3-K p85 subunit, Src family kinases, adaptor molecules (Shc, Crk, Nck, Grb2) and Ras-GAP. SH2/SH3 domains as targets for anti-cancer drugs are discussed in Smithgall, T. E. (1995), Journal of Pharmacological and Toxicological Methods. 34(3) 125-32.

Inhibitors of Serine/Threonine Kinases including MAP kinase cascade blockers which include blockers of Raf kinases (rafk), Mitogen or Extracellular Regulated Kinase (MEKs), and Extracellular Regulated Kinases (ERKs); and Protein kinase C family member blockers including blockers of PKCs (alpha, beta, gamma, epsilon, mu, lambda, iota, zeta). IkB kinase family (IKKa, IKKb), PKB family kinases, akt kinase family members, and TGF beta receptor kinases. Such Serine/Threonine kinases and inhibitors thereof are described in Yamamoto, T., Taya, S., Kaibuchi, K., (1999), Journal of Biochemistry. 126 (5) 799-803; Brodt, P, Samani, A., and Navab, R. (2000), Biochemical Pharmacology, 60. 1101-1107; Massague, J., Weis-Garcia, F. (1996) Cancer Surveys. 27:41-64; Philip, P. A., and Harris, A. L. (1995), Cancer Treatment and Research. 78: 3-27, Lackey, K. et al Bioorganic and Medicinal Chemistry Letters, (10), 2000, 223-226; U.S. Pat. No. 6,268,391; and Martinez-Iacaci, L., et al, Int. J. Cancer (2000), 88(1), 44-52.

Inhibitors of Phosphotidyl inositol-3 Kinase family members including blockers of PI3-kinase, ATM, DNA-PK, and Ku are also useful in the present invention. Such kinases are discussed in Abraham, R. T. (1996), Current Opinion in Immunology. 8 (3) 412-8; Canman, C. E., Lim, D. S. (1998), Oncogene 17 (25) 3301-3308; Jackson, S. P. (1997), International Journal of Biochemistry and Cell Biology. 29 (7):935-8; and Zhong, H. et al, Cancer res, (2000) 60(6), 1541-1545.

Also useful in the present invention are Myo-inositol signaling inhibitors such as phospholipase C blockers and Myoinositol analogues. Such signal inhibitors are described in Powis, G., and Kozikowski A., (1994) New Molecular Targets for Cancer Chemotherapy ed., Paul Workman and David Kerr, CRC press 1994, London.

Another group of signal transduction pathway inhibitors are inhibitors of Ras Oncogene. Such inhibitors include inhibitors of farnesyltransferase, geranyl-geranyl transferase, and CAAX proteases as well as anti-sense oligonucleotides, ribozymes and immunotherapy. Such inhibitors have been shown to block ras activation in cells containing wild type mutant ras, thereby acting as antiproliferation agents. Ras oncogene inhibition is discussed in Scharovsky, O. G., Rozados, V. R., Gervasoni, S. I. Matar, P. (2000), Journal of Biomedical Science. 7(4) 292-8; Ashby, M. N. (1998), Current Opinion in Lipidology. 9 (2) 99-102; and BioChim. Biophys. Acta, (19899) 1423(3):19-30.

As mentioned above, antibody antagonists to receptor kinase ligand binding may also serve as signal transduction inhibitors. This group of signal transduction pathway inhibitors includes the use of humanized antibodies to the extracellular ligand binding domain of receptor tyrosine kinases. For example Imclone C225 EGFR specific antibody (see Green, M. C. et al, Monoclonal Antibody Therapy for Solid Tumors, Cancer Treat. Rev., (2000), 26(4), 269-286); Herceptin® erbB2 antibody (see Tyrosine Kinase Signalling in Breast cancer:erbB Family Receptor Tyrosine Kinases, Breast cancer Res., 2000, 2(3), 176-183); and 2CB VEGFR2 specific antibody (see Brekken, R. A. et al, Selective Inhibition of VEGFR2 Activity by a monoclonal Anti-VEGF antibody blocks tumor growth in mice, Cancer Res. (2000) 60, 5117-5124).

Anti-angiogenic agents: Anti-angiogenic agents including non-receptorMEKngiogenesis inhibitors may also be useful. Anti-angiogenic agents such as those which inhibit the effects of vascular endothelial growth factor, (for example the anti-vascular endothelial cell growth factor antibody bevacizumab [Avastin™], and compounds that work by other mechanisms (for example linomide, inhibitors of integrin αvβ3 function, endostatin and angiostatin);

Immunotherapeutic agents: Agents used in immunotherapeutic regimens may also be useful in combination with the compounds of formula (I). Immunotherapy approaches, including for example ex-vivo and in-vivo approaches to increase the immunogenecity of patient tumour cells, such as transfection with cytokines such as interleukin 2, interleukin 4 or granulocyte-macrophage colony stimulating factor, approaches to decrease T-cell anergy, approaches using transfected immune cells such as cytokine-transfected dendritic cells, approaches using cytokine-transfected tumour cell lines and approaches using anti-idiotypic antibodies

Proapoptotoc agents: Agents used in proapoptotic regimens (e.g., bcl-2 antisense oligonucleotides) may also be used in the combination of the present invention.

Cell cycle signalling inhibitors: Cell cycle signalling inhibitors inhibit molecules involved in the control of the cell cycle. A family of protein kinases called cyclin dependent kinases (CDKs) and their interaction with a family of proteins termed cyclins controls progression through the eukaryotic cell cycle. The coordinate activation and inactivation of different cyclin/CDK complexes is necessary for normal progression through the cell cycle. Several inhibitors of cell cycle signalling are under development. For instance, examples of cyclin dependent kinases, including CDK2, CDK4, and CDK6 and inhibitors for the same are described in, for instance, Rosania et al, Exp. Opin. Ther. Patents (2000) 10(2):215-230.

In one embodiment, the combination of the present invention comprises an anti-OX40 ABP and a PD-1 modulator (e.g. anti-PD-1 ABP) and at least one anti-neoplastic agent selected from anti-microtubule agents, platinum coordination complexes, alkylating agents, antibiotic agents, topoisomerase II inhibitors, antimetabolites, topoisomerase I inhibitors, hormones and hormonal analogues, signal transduction pathway inhibitors, non-receptor tyrosine MEKngiogenesis inhibitors, immunotherapeutic agents, proapoptotic agents, and cell cycle signaling inhibitors.

In one embodiment, the combination of the present invention comprises an anti-OX40 ABP and a PD-1 modulator (e.g. anti-PD-1 ABP) and at least one anti-neoplastic agent which is an anti-microtubule agent selected from diterpenoids and vinca alkaloids.

In a further embodiment, the at least one anti-neoplastic agent is a diterpenoid.

In a further embodiment, the at least one anti-neoplastic agent is a vinca alkaloid.

In one embodiment, the combination of the present invention comprises an anti-OX40 ABP and a PD-1 modulator (e.g. anti-PD-1 ABP) and at least one anti-neoplastic agent, which is a platinum coordination complex.

In a further embodiment, the at least one anti-neoplastic agent is paclitaxel, carboplatin, or vinorelbine.

In a further embodiment, the at least one anti-neoplastic agent is carboplatin.

In a further embodiment, the at least one anti-neoplastic agent is vinorelbine.

In a further embodiment, the at least one anti-neoplastic agent is paclitaxel.

In one embodiment, the combination of the present invention comprises an anti-OX40 ABP and a PD-1 modulator (e.g. anti-PD-1 ABP) and at least one anti-neoplastic agent which is a signal transduction pathway inhibitor.

In a further embodiment the signal transduction pathway inhibitor is an inhibitor of a growth factor receptor kinase VEGFR2, TIE2, PDGFR, BTK, erbB2, EGFr, IGFR-1, TrkA, TrkB, TrkC, or c-fms.

In a further embodiment the signal transduction pathway inhibitor is an inhibitor of a serine/threonine kinase rafk, akt, or PKC-zeta.

In a further embodiment the signal transduction pathway inhibitor is an inhibitor of a non-receptor tyrosine kinase selected from the src family of kinases.

In a further embodiment the signal transduction pathway inhibitor is an inhibitor of c-src.

In a further embodiment the signal transduction pathway inhibitor is an inhibitor of Ras oncogene selected from inhibitors of farnesyl transferase and geranylgeranyl transferase.

In a further embodiment the signal transduction pathway inhibitor is an inhibitor of a serine/threonine kinase selected from the group consisting of PI3K.

In a further embodiment the signal transduction pathway inhibitor is a dual EGFr/erbB2 inhibitor, for example N-{3-Chloro-4-[(3-fluorobenzyl) oxy]phenyl}-6-[5-({[2-(methanesulphonyl) ethyl]amino}methyl)-2-furyl]-4-quinazolinamine (structure below):

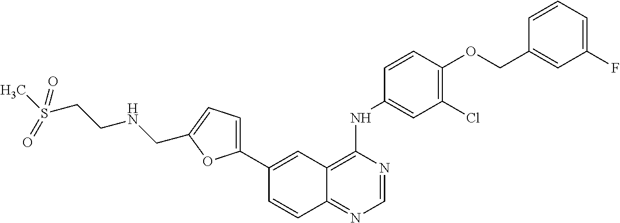 [SMILES: CS(=O)(=O)CCNCc1ccc(-c2ccc3ncnc(Nc4ccc(OCc5cccc(F)c5)c(Cl)c4)c3c2)o1]

In one embodiment, the combination of the present invention comprises a compound of formula I or a salt or solvate thereof and at least one anti-neoplastic agent which is a cell cycle signaling inhibitor.

In further embodiment, cell cycle signaling inhibitor is an inhibitor of CDK2, CDK4 or CDK6.

In one embodiment the mammal in the methods and uses of the present invention is a human.

As indicated, therapeutically effective amounts of the combinations of the invention (an anti-OX40 ABP and a PD-1 modulator (e.g. anti-PD-1 ABP)) are administered to a human. Typically, the therapeutically effective amount of the administered agents of the present invention will depend upon a number of factors including, for example, the age and weight of the subject, the precise condition requiring treatment, the severity of the condition, the nature of the formulation, and the route of administration. Ultimately, the therapeutically effective amount will be at the discretion of the attendant physician.

The following examples are intended for illustration only and are not intended to limit the scope of the invention in any way.

### EXAMPLES

### Example 1

### Introduction

CT-26 is an N-nitroso-N-methylurethane-(NNMU) induced, undifferentiated murine colon carcinoma cell line in Balb/c mice. CT26 cells readily establish tumors in syngenic mice, producing histologically proven adenocarcinoma with a predictable growth rate in a reasonable time frame. In addition, CT26 tumor growth induced an extensive cellular immune response of predominantly regulatory T cells with a presumably protumoral activity. CT26 model has been widely used to evaluate the antitumor immune response of Immunotherapeutics. In these experiments, therapeutic use of the mouse analog of anti OX-40, OX-86, was evaluated in this mouse tumor model both independently and in combination with anti mouse PD-1 (RMP1-14 clone).

### Methods

### Experimental Preparation(s)

All procedures on animals were reviewed and approved by the GSK Institutional Animal Care and Use Committee prior to initiation of the studies.

A frozen (−140° C.) vial of CT26, mouse colon carcinoma cells, from ATCC (cat# CRL-2638, lot#59227052) was thawed and the cells cultured according to the supplier's recommendations, approximately a week (three passages from thaw) before inoculation.

In the week prior to inoculation, 6-8 week old female Balb/c mice from Charles River were individually labelled via alpha-numerical tattoo on the tail or subcutaneously injected identification chip (BMDS, IMI-1000 transponder).

### Experimental Protocol(s)

The purpose of these experiments is to evaluate anti-tumor therapeutics in mouse syngeneic tumorgenesis models. Animals are weighed and inoculated on the right hind quarter with 100 ul of 0.5×10⁵ CT26 tumor cells per mouse on Day 0. The number of mice inoculated is equal to 130% of what is needed for the study. Assuming 30% failure rate (either too big or too small at time of start of study), the goal is to have n=10 for each group. After tumor cell inoculation, tumor growth and total body weight are measured 3 times a week with a Fowler “ProMax” digital caliper for 4 weeks or longer. Antibodies were acquired from commercial vendor and diluted to desired concentration in 0.9% saline. Dosing (i.p.) occurs biweekly, for a total of 6 doses and initiates on the day of randomization when average tumor volume approximates 100 mm³, likely day 10 or 11. Randomization is performed using the Studylog Study Director Suite software. Length and width of tumors are measured in order to determine tumor volume using the formula (tumor volume=L×W²/2). Tumor measurement of greater than 2,000 mm³ for an individual animal results in euthanization. Mice may also be removed from the study due to weight loss (>20%), ulceration or tumor necrosis, or any other obvious inhibition of normal mouse activity due to morbidity.

### Combination Therapy; Anti PD-1 (RMP1-14)/OX86

A total of 130 mice were inoculated with CT-26 cell in order to provide for a study with ten groups of n=10. The purpose of this study was to evaluate PD-1 (RMP1-1) at 200 ug per mouse in combination with various doses of OX-40 (OX86) to determine if the efficacy seen in N31299-6 was reproducable. Isotypes for anti PD-1 (rat IgG2a) and OX86 (ratIgG1) were evaluated individually and in combination. Monotherapies of anti PD-1 and OX86 were evaluated in concert with the isotype controls for its counterpart in combination therapy. A dose level of 200 ug per mouse for anti PD-1 was evaluated in combination with 5, 20, 100, and 400 ug per mouse of OX86. Statistical analysis of tumor volume was performed on day 21 (day 12 post randomization). Survivability analysis concludes upon termination of the study, day 89.

| Dosing | treatment 1 | treatment 2 | n = |
|---|---|---|---|
| Group 1: 0.5 × 10⁵ cells per, | saline | none | 10 |
| Group 2: 0.5 × 10⁵ cells per, | RatIgG1 400 ug | none | 10 |
| Group 3: 0.5 × 10⁵ cells per, | RatIgG2a 200 ug | none | 10 |
| Group 4: 0.5 × 10⁵ cells per, | RatIgG1 400 ug | RatIgG2a 200 ug | 10 |
| Group 5: 0.5 × 10⁵ cells per, | aPD-1 200 ug | RatIgG1 400 ug | 10 |
| Group 6: 0.5 × 10⁵ cells per, | O × 86 400 ug | RatIgG2a 200 ug | 10 |
| Group 7: 0.5 × 10⁵ cells per, | O × 86 5 ug | aPD-1 200 ug | 10 |
| Group 8: 0.5 × 10⁵ cells per, | O × 86 20 ug | aPD-1 200 ug | 10 |
| Group 9: 0.5 × 10⁵ cells per, | O × 86 100 ug | aPD-1 200 ug | 10 |
| Group 10: 0.5 × 10⁵ cells per, | O × 86 400 ug | aPD-1 200 ug | 10 |

### 1.1. Data Analysis

The event for survival analysis is tumor volume of 2000 mm³ or tumor ulceration, whichever came first. The exact time to cut-off volume was estimated by fitting a linear line between log tumor volume and day of two observations, the first observation that exceed the cut-off volume and the one observation that immediately preceded the cut-off volume. Kaplan-Meier (KM) method was carried out to estimate the survival probability of different treatment groups at a given time. The median time to endpoint and its corresponding 95% confidence interval was reported. Whether or not KM survival curves are statistically different between any two groups was then tested by log-rank test.

Tumor volume data from the last day in which there are 10 animals per group (i.e. before any animals are euthanized) were compared between the different treatment groups. Prior to the analysis, the tumor volume was natural log transformed due to the inequality of variance in the different treatment groups. ANOVA followed by pair-wise comparison were then carried out on the log transformed data.

### 1.2. Combination Therapy; Anti PD-1 (RMP1-14)/OX86

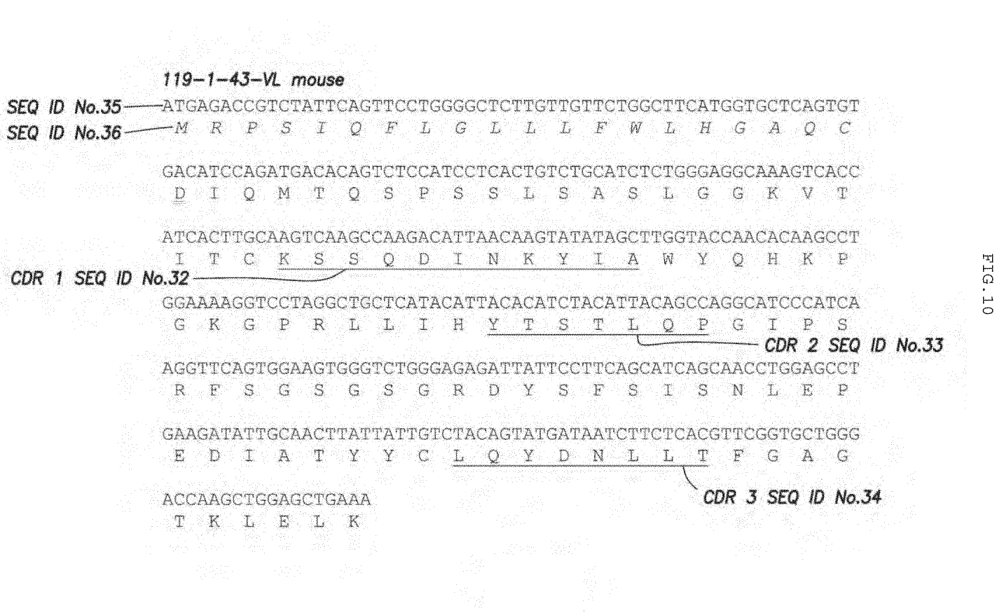

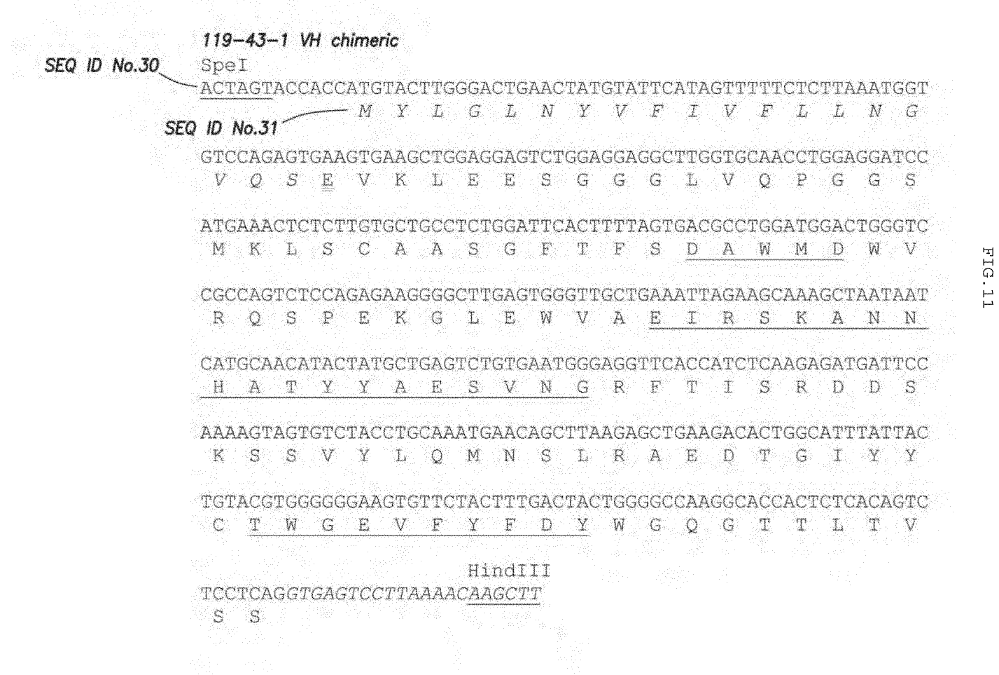

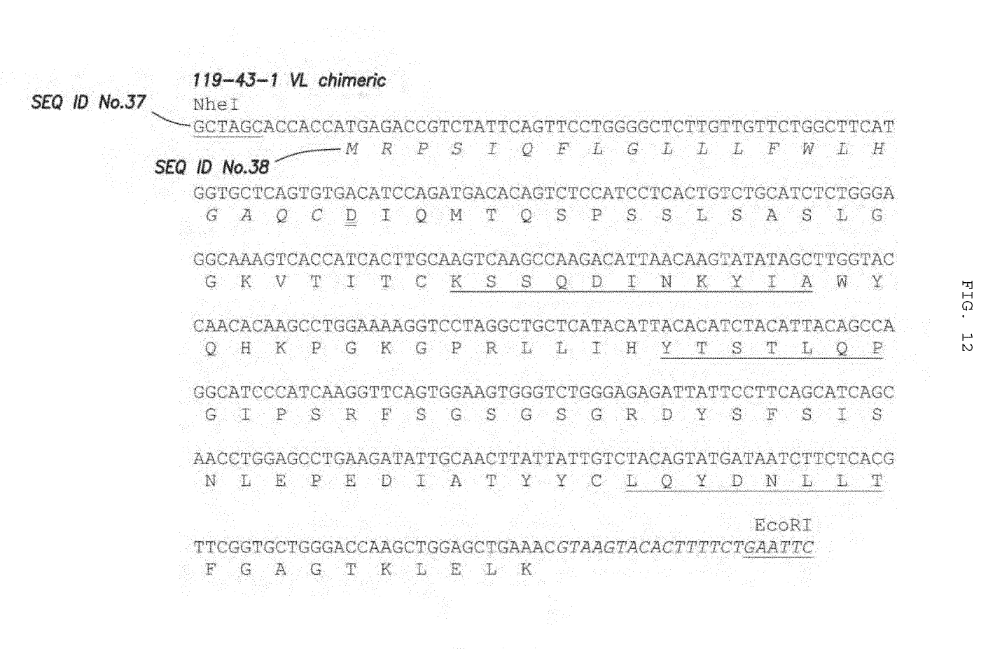

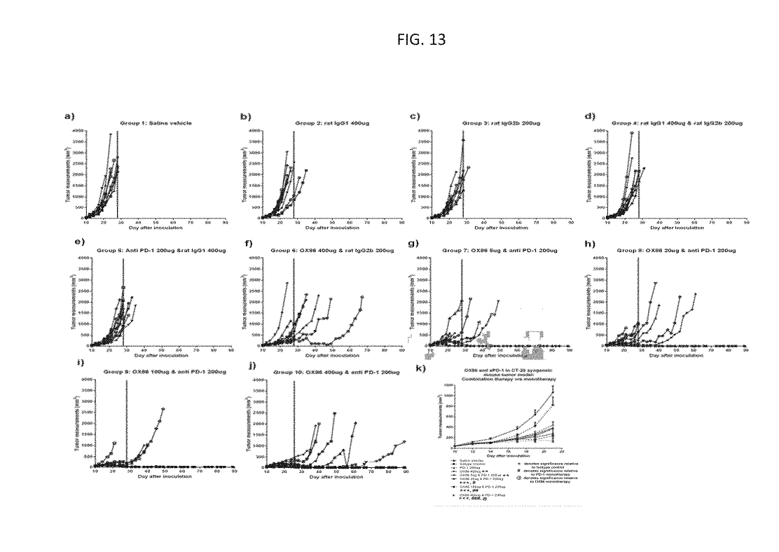

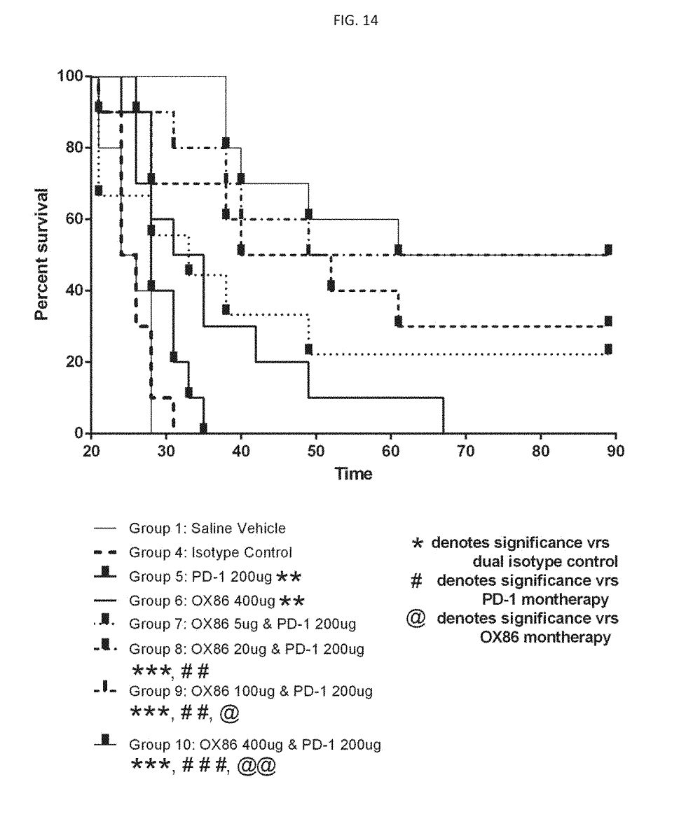

Among groups that received either saline, individual isotype controls or dual isotype control treatment, there was no significant or otherwise observable difference in tumor growth at day 21 (FIG. 13, panel k) or survivability (FIG. 14). When compared to saline control, PD-1 alone demonstrated efficacy in both tumor growth reduction at day 21 (p<0.01) and survivability (p<0.001). When compared to the dual isotype control, dosing with PD-1 alone resulted in a statistically significant difference in survivability (p<0.01) only (FIG. 14). Monotherapy of OX86 demonstrated statistical significance in both tumor growth inhibition by day 21 and survivability when compared to saline control (p<0.01 for both) and dual isotype control (p<0.01 for both) (FIG. 13, panel k and FIG. 14). When compared to saline and dual isotype controls, the combination therapy of PD-1 and 5 ug OX86 resulted in a reduction in tumor growth through day 21 (p<0.001 and p<0.01, respectively) (FIG. 13, panel k) and increase in survivability (p<0.05 and ns, respectively) (FIG. 14). The results of dual therapies with higher doses of OX86 were more drastic. There was a considerable decrease in tumor growth through day 21 (FIG. 13, panel k) and increase in survivability (FIG. 14) at 20, 100 and 400 ug doses of OX86 when compared to both saline and isotype controls (p<0.001 for all). When compared to the PD-1 monotherapy, there was no statistically significant increase in efficacy by the combination therapy of PD-1 and 5 ug OX86. However, at OX86 doses of 20, 100, and 400 ug there were statistically significant decreases in tumor growth through day 21 (p<0.05, p<0.01, and p<0.001, respectively) (FIG. 13, panel k) and increases in survivability (p<0.01, p<0.01, and p<0.001, respectively) (FIG. 14). When compared to the 400 ug OX86 monotherapy, there was significance in the decrease in average tumor growth through day 21 (p<0.05) (FIG. 13, panel k) and increase in survivability (p<0.01) (FIG. 14) in the combination therapy at the 400 ug dose of OX86. The combination therapy that included 100 ug dose of OX86 also resulted in a statistically significant increase in survivability (p<0.05) and a non-significant yet observable decrease in average tumor growth at day 21. At the conclusion of this study there were 13 mice with tumors less than 10 mm3 in volume. All of these animals were in combination groups; 2 in the 5 ug OX86/PD-1 (FIG. 13, panel g), 3 in the 20 ug OX86/PD-1 (FIG. 13, panel h), 4 in the 100 ug OX86/PD-1 (FIG. 13, panel i), 4 in the 400 ug OX86/PD-1 (FIG. 13, panel j).

FIG. 14 shows results from a study wherein mice were inoculated with 0.5e5 CT-26 cells per mouse on day 0. Animals were randomized and dosing began on day 10 after inoculation and continued twice a week for 3 weeks (total of 6 doses). Vehicle control group was dosed i.p. with saline and the isotype control received i.p. doses of the isotypes for OX86 (rat IgG1) and anti PD-1 RMP1-14 (rat IgG2a), both individually and in combination. Monotherapy animals were dosed with 200 ug of anti PD-1 and 400 ug rat IgG1 or 20 or 400 ug of OX86 and 200 ug rat IgG2a. Combination therapy mice received 200 ug of PD-1 and 5, 20, 100 or 400 ug of OX86. Average tumor growth was analyzed at day 21 post inoculation. All combination therapies significantly reduced average tumor growth by day 21 when compared to the isotype control. All but the 5 ug combination therapy reduced average tumor growth relative to the PD-1 monotherapy. The 400 ug OX86 combination therapy resulted in a reduction in average tumor size at day 21 relative to both monotherapies. The 20, 100 and 400 ug OX86 combination therapies all resulted in a significant increase in survivability relative to the isotype control and PD-1 monotherapy. The 100 ug and 400 ug OX86 combination therapy significantly increased survivability compared to the OX86 monotherapy. (*p<0.05, **p<0.01, ***p<0.001; ANOVA followed by pair-wise comparison were carried out on natural log transformed tumor volume data, log-rank test was used to determine significance in KM survival curves).

Conclusion: This study examined OX86 in combination with anti-mouse PD-1. In comparing the PD-1 monotherapy with combination groups, all OX86+PD-1 combination groups, except for the group with a low dose of 5 ug OX86, showed a significant increase in survival compared to PD-1. In comparing the 400 ug OX86 monotherapy to combination groups, the 100 and 400 ug OX86+PD-1 combination groups showed a significant increase in survival compared to OX86 monotherapy. Thus, combinations of OX86 and PD-1 showed a significant increase in survival compared to PD-1 and OX86 monotherapies.

### Example 2

### OX-401 PD-1 CT-26 Rechallenge Study

### Methods

A frozen (−140° C.) vial of CT-26 mouse colon carcinoma cells (ATCC cat# CRL-2638, lot#59227052) was thawed and cultured in complete medium (RPMI with 10% FBS). Cells were harvested from the flask in complete medium, centrifuged and resuspended again in complete medium. This step was repeated 3 times in RPMI media without FBS. Cell density and viability were checked via trypan blue exclusion either through the use of hemocytometer or Vi-CELL. Cells were then diluted to 5×10⁵ cells per mL for inoculation in PBS.

One hundred and thirty female BALB/c mice, aged 6 to 7 weeks, that had been previously injected subcutaneously between the shoulders with IMI-1000 transponders were inoculated with 100 ul (microliters) of the 5×10⁵ cells per mL suspension. Injections were performed subcutaneously on the right hind quarter of the mouse with a 25 G needle. An additional 13 mice were left uninoculated until the time of re-challenge to serve as an age-matched control. After tumor cell inoculation, tumor growth was measured using a Fowler “ProMax” digital caliper. Length and width of tumors were measured in order to determine tumor volume using the formula (tumor volume=L×W²×0.52). Mice were randomized using StudyLog Study Director Suite software on day 13 post inoculation when the average tumor size was approximately 100 mm³. Intraperitoneal dosing, beginning on day 14 post inoculation, occurred biweekly for a total of 6 doses. Tumor measurement of greater than 2,000 mm³ for an individual animal resulted in euthanization. Mice were also removed from the study due to weight loss (>20%), ulceration or tumor necrosis, or any other obvious inhibition of normal mouse activity due to morbidity. On day 44 post inoculation, all mice remaining on study with measurable tumors were euthanized. On day 71 post inoculation, all remaining mice with completely regressed tumors, along with the 13 naive mice were inoculated on the left flank with 0.5×10⁵ CT-26 cells using the same method described above.

All antibodies were diluted to desired concentrations in 0.9% sodium chloride (Hospira, NDC 0409-4888-10, lot #44-324-DK) on the day of dosing and injected intraperitoneally in a volume of 0.2 mL per mouse using a 30 G needle. For this experiment, OX86 and its isotype control were dosed at 100 ug (micrograms) per mouse and PD-1 and its isotype control were dosed at 200 ug per mouse. The mice receiving the OX86 monotherapy also received the isotype control for PD-1, rat IgG2a. Similarly, the mice being dosed with the PD-1 monotherapy were also dosed with the isotype control for OX86, rat IgG1. The isotype group received both isotype controls and served as an isotype control. The vehicle control mice were dosed with 0.9% saline.

| Antibody | Supplier | Cat # | Lot # | Conc. (mg/mL) |
|---|---|---|---|---|
| Rat IgG1 | Bioxcell | BE0088 | 5339/0814 | 7.86 |
| O × 86 (Hybridoma CD134) | Bioxcell | BE0031 | 5534/01214 | 7.75 |
| Rat IgG2a | Bioxcell | BE0089 | 4620-2/0513 | 8.98 |
| Anti-m PD-1 (RMP1-14) | Bioxcell | BE0147 | 5311-4/0115 | 7.75 |
| pembrolizumab | Sourced via | NDC0006- | L010592 | 25 mg/mL |
| (KEYTRUDA ®) | Clinical and | 3026-02 | | |
| | Investigational | | | |
| | Material | | | |
| | Supply | | | |
| | organization | | | |

### Evaluating Pharmacodynamic Changes in Markers:

Female BALB/c (8-10 wks old from Charles River Laboratories) mice were implanted with 0.5×10⁵ CT-26 colon carcinoma cells subcutaneously on their flanks. Mice were randomized and treatment was started when the tumors reached approximately 100 mm³. The mice were randomized into five groups receiving vehicle, isotype control antibodies (IgG1 100 ug+IgG2a 200 ug), anti-OX40 (OX86 100 ug+IgG2a 200 ug), anti-PD-1 (PD-1 200 ug+IgG1 100 ug) or anti-OX40 and anti-PD-1 (OX86100 ug+PD-1 200 ug). The mice were dosed twice a week on day 0, 3 and 7 and harvested on day 3, 7 and 10 following the first dose. Mice collected on day 3, 7 and 10 received 1, 2 and 3 doses respectively. Serum was collected for cytokine analysis and analyzed by Meso-Scale Discovery mouse V-Plex customized kit. Blood and tumor samples were collected, and following dissociation of the tumors, both the tissues were stained with antibodies for flow cytometric analysis. Data was acquired on BD FACSCanto II. The data were analyzed by FlowJo software and statistical analysis was carried out by one way ANOVA using Kruskal-Wallis test. Gene expression in tumors was analyzed by NanoString technology.

### Tumor Isolation:

Excised tumors were mechanically dissociated in a 60 mm petri dish on ice with 2 mL of PBS and were transferred to 5-15 mL polypropylene tubes. Miltenyi Tumor Dissociation Kit cocktail (Miltenyi Biotec, Cat#130-096-730) of enzymes was prepared in RPMI as per the kit protocol and added to each tube (˜2.5 mL per tumor). Digestion was allowed to proceed for 40 minutes at 37° C. After digestion, the remaining pieces and the suspension were filtered through 30-100 um (micron) cell strainers into a 50 mL tube. The strainers were washed with 10 mL PBS and brought up to 20 mL. Tubes containing cells were centrifuged at 300 rcf for 5 minutes, supernatant aspirated, and pellet resuspended in 5 mL PBS.

Lymphocytes and tumor cells were counted and total cell number was estimated using Vi-CELL. Cells were diluted to a final concentration of 1×10⁶ cell/100 uL and stained with antibodies.

### Flow Cytometric Analysis:

400 ul of blood was transferred into 8 mL ACK lysis buffer (Thermo Fisher Scientific) in a 15 mL conical tube, mixed well and incubated at room temperature for 5 minutes. Tubes were centrifuged at 3500×g for 10 minutes at 10° C. and supernatant aspirated. The pellet was resuspended in 10 mL of FACS buffer (PBS with 0.5% BSA) and centrifuged at 2700×g for 5 minutes at 10° C. Supernatants were aspirated, pellets resuspended in 10 mL FACS buffer and tubes spun at 1600 rpm (IEC CL31R) for 5 min at 10° C. The wash was repeated with FACS buffer. Cell pellets were resuspended in 800 ul FACS buffer. 100 ul cell suspension of either cells isolated from tumor, spleen or blood were aliquoted into each 96-well plate, and the plate was centrifuged at 1600 rpm (IEC CL31R) for 5 minutes. The supernatants were removed. Cells were blocked with 50 ul of FACS buffer plus 2% rat serum or FcR Blocking Reagent (Miltenyi Biotec, Cat#130-092-575) for 10 minutes on ice. Cells were stained by adding desired dilution of antibodies in 50 ul of FACS buffer and incubating on ice for 30 minutes.

Intracellular staining was carried out by adding 200 uL of Foxp3 Fixation/Permeabilization (BD Biosciences, Cat#554714) working solution was added to each well, and cells were resuspended. Cells were incubated in the dark at 4° C. for 30 minutes. Plates were centrifuged at room temperature for 5 minutes at 400×g, and the supernatant was discarded. Cells were washed twice in 200 uL of 1× Permeabilization Buffer (BD Biosciences, Cat#554714). Cells were blocked with 2% rat serum/1% hamster serum and incubated at room temperature for 15 minutes. Without washing, the recommended amount of fluorochrome-conjugated antibody was added for detection of intracellular antigen(s), and plates were incubated in the dark at room temperature for at least 30 minutes. Cells were washed twice in 200 μL of 1× Permeabilization Buffer. Stained cells were resuspended in an appropriate volume of FACS buffer and stored at 4° C. until acquisition on the flow cytometer the following day.

### Mouse T-Cell Receptor Sequencing:

Female BALB/c (8-10 wks old from Charles River Laboratories) mice were implanted with 0.5×10⁵ CT-26 colon carcinoma cells subcutaneously on their flanks. Mice were randomized and treatment was started when the tumors reached approximately 100 mm³. The mice were randomized into four groups receiving isotype (IgG1 100 ug+IgG2a 200 ug), agonistic anti-OX40 (OX86 100 ug+IgG2a 200 ug), anti-PD-1 (anti-PD-1 200 ug+IgG1 100 ug) or anti-OX40 and anti-PD-1(OX86 100 ug+PD-1 200 ug) antibodies. Each group of 7 mice were dosed twice a week. 72 hours post the third dose, the mice were euthanized, and blood was collected in EDTA blood collection tubes (BD Microtainer Cat#365974), while the tumors were collected in 1.8 ml cryotubes flash frozen. Samples were shipped to Adaptive Biotechnologies for TCR analysis.

### In Vitro Studies:

PBMCs from healthy human donors were cultured with anti-CD3/anti-CD28 beads at beads to cells ratio of 1:20, IL-2 and MCSF in AIM-V media for 48 hours. The cells were then restimulated with anti-CD3 beads at beads to cells ratio of 1:1, ANTIBODY 106-222 (aOX40), pembrolizumab (aPD-1) or both at 10 ug/ml for 72 hrs. Cell supernatants were collected and analyzed for IFNγ and TNFα by Meso Scale Discovery. Unpaired t test for statistical analysis was used. **=p<0.005, ***<p 0.0003. Data is representative one of five donors.

### Cytokine Profiles

Serum cytokine levels were tested using the Meso-Scale Discovery mouse V-flex customized kit. Samples and calibrators were diluted 1:2 in Diluent 41 as per the kit manual. 50 ul of prepared samples and calibrators were added to the MSD plate each in triplicate. Plates were sealed and incubated at room temperature with shaking for 2 hours. Plates were washed 3 times with 150 u/well of PBS plus 0.05% Tween-20. 25 uL of detection antibody solution prepared in Diluent 45 was added to each well. Plates were sealed and incubated at room temperature with shaking for 2 hours. Plates were washed as above. 150 ul/well of freshly diluted 2× read buffer was added to the plates which were immediately read on MESO QuickPlex reader. Data were analyzed using MSD Workbench software and graphed using GraphPad Prism.

### Conclusions:

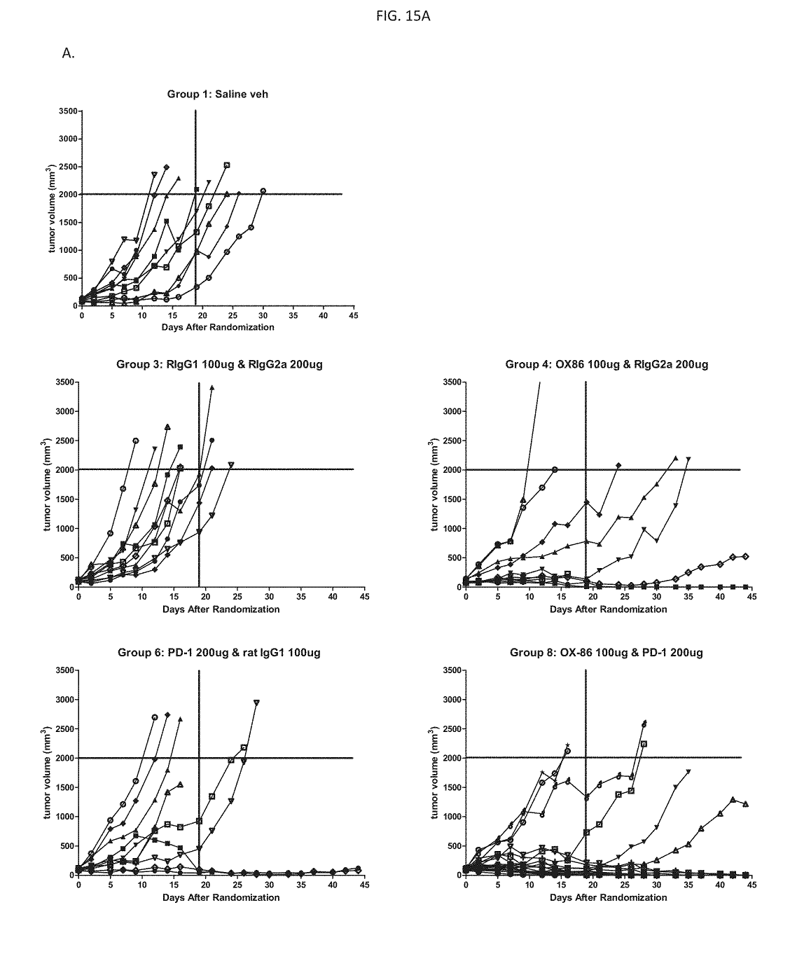

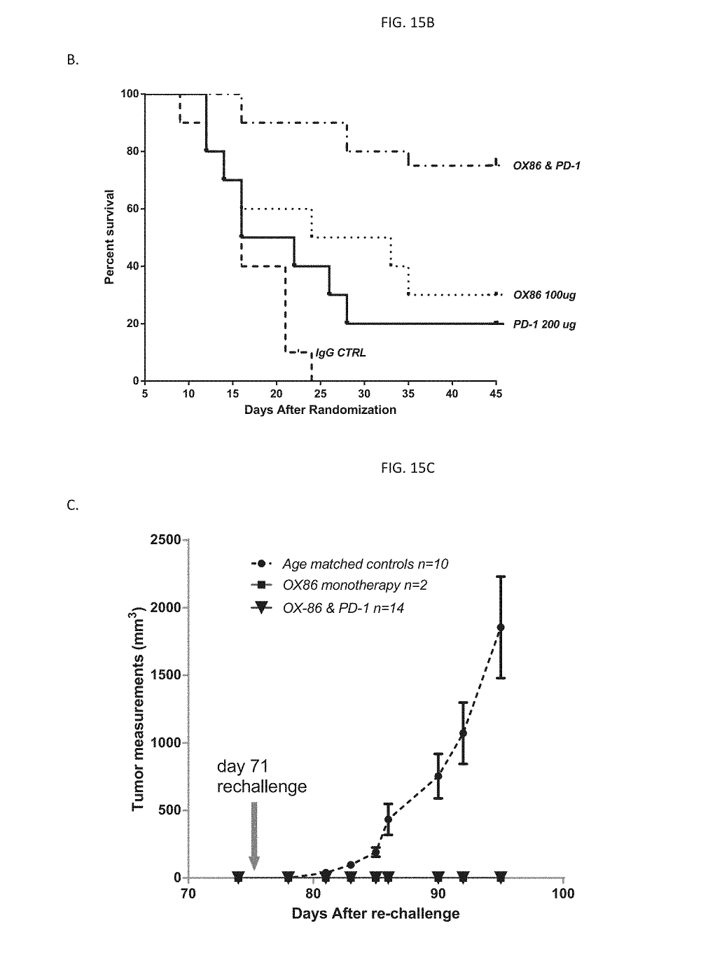

Re-challenge study in CT-26 model
Combination therapies resulted in higher tumor regression rate and survivability than that seen in monotherapies (FIG. 15A)
   0 out of 10 in PD-1 monotherapy group demonstrated total tumor regression
   2 out of 10 in OX86 monotherapy group
   14 out of 20 in PD-1/OX86 treatment group
Mice cured with either OX86 or OX86/PD1 are completely protected from CT-26 tumor rechallenge as demonstrated by total tumor loss compared to control group (FIG. 15B and FIG. 15C)

### PD Data Cytokine Analysis:

a. Th1 cytokine IFNγ are significantly upregulated in the group receiving combination of anti-OX40+anti-PD-1, 7 days following treatment. Similar trends are observed on day 10 as well. TNFα is significantly upregulated in the combination cohort.

### PD Immunophenotyping Analysis:

a. There is a significant increase in the proliferation of CD4 T-cells in the blood at all day 3 and day 7 following anti-OX40 treatment, while anti-OX40+anti-PD-1 treatment demonstrated a significant increase at day 3 and day 10. There is a significant increase in the CD8 T-cells at day 7 and day 10 following treatment with anti-OX40 and anti-PD-1 combination treatment.
b. There is a significant increase in the effector memory CD4 T cells at day 7 and day 10 following the combination monoclonal antibody treatment. Combination treatment results in a significant increase in the CD8 effector memory cells at day 3 and day 7.
c. There is a significant increase in the proportion of activated CD25^{+ive} CD8 T cells in group treated with anti-OX40 and anti-PD1 at 7 and day 10 post initiation of treatment. Anti-OX40 monotherapy significantly increases the CD25^{+ive} CD8 T cells at day 7. While not significant, there is a trend of increase of CD8 T cells expressing PD1 in both anti-OX40 and combination treated groups at day 7 and day 10 in circulation.
d. In the tumors there is a significant increase in the cytotoxic granzyme B expressing CD8 T cells in the combination treated groups at both day 7 and day 10. At day 10 the CD8: Treg ratio is significantly increased in the group receiving combination treatment.

### Nanostring Analysis:

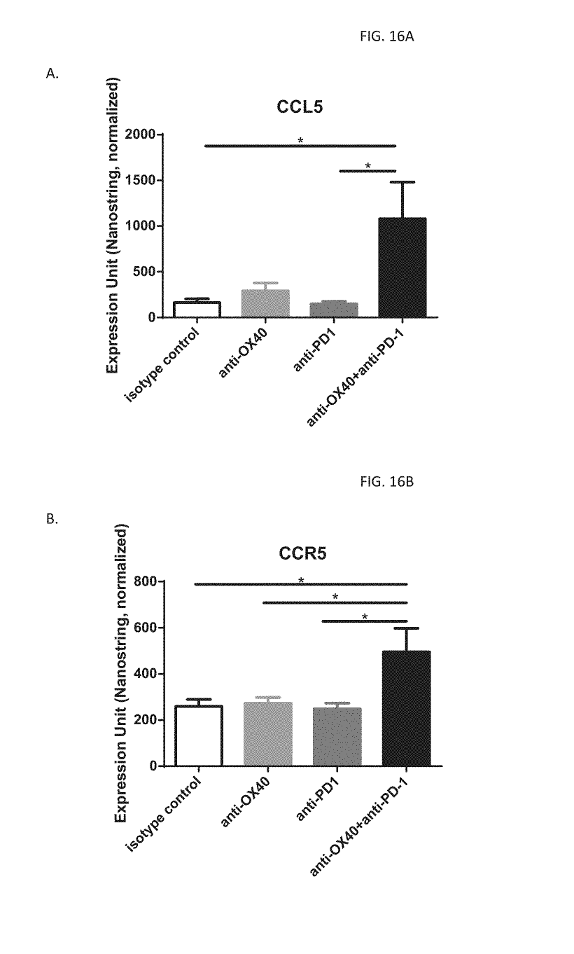

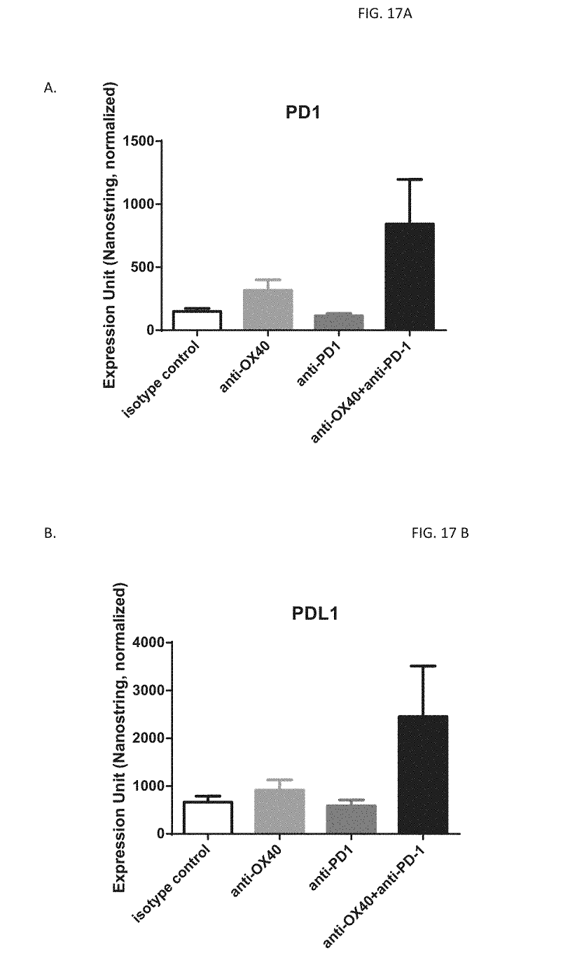

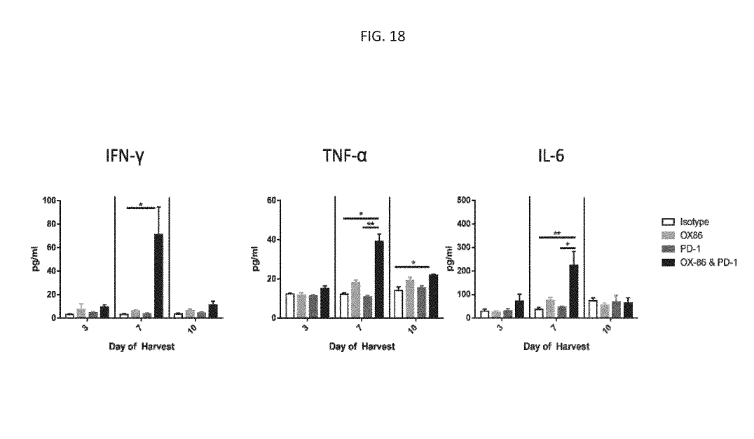

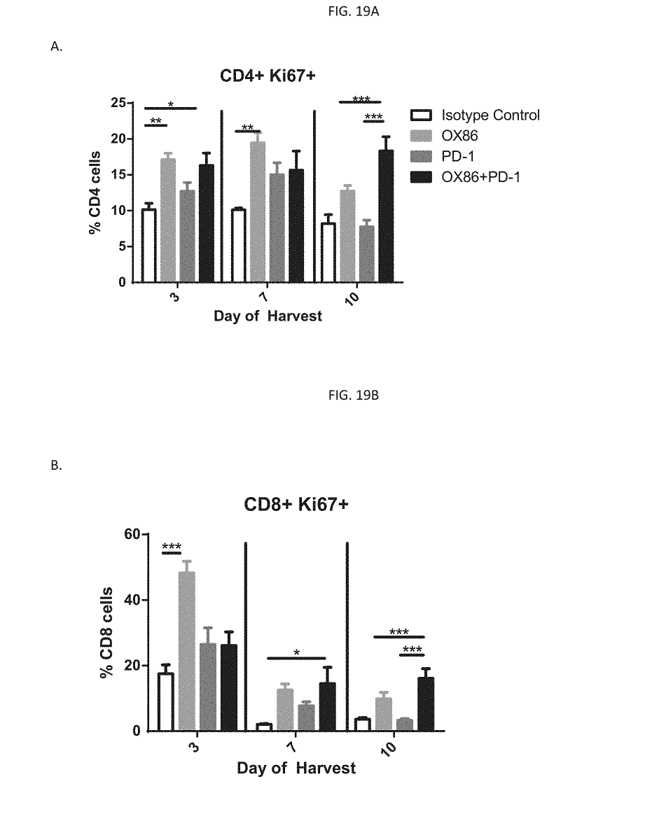

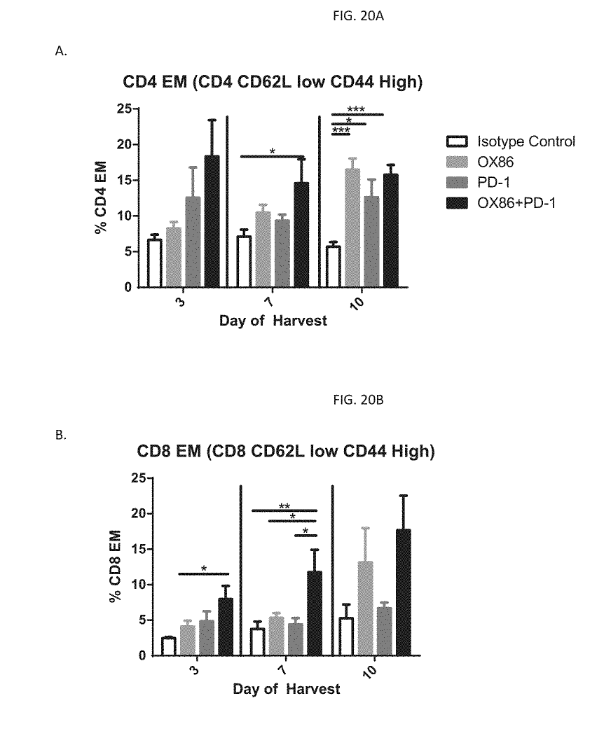

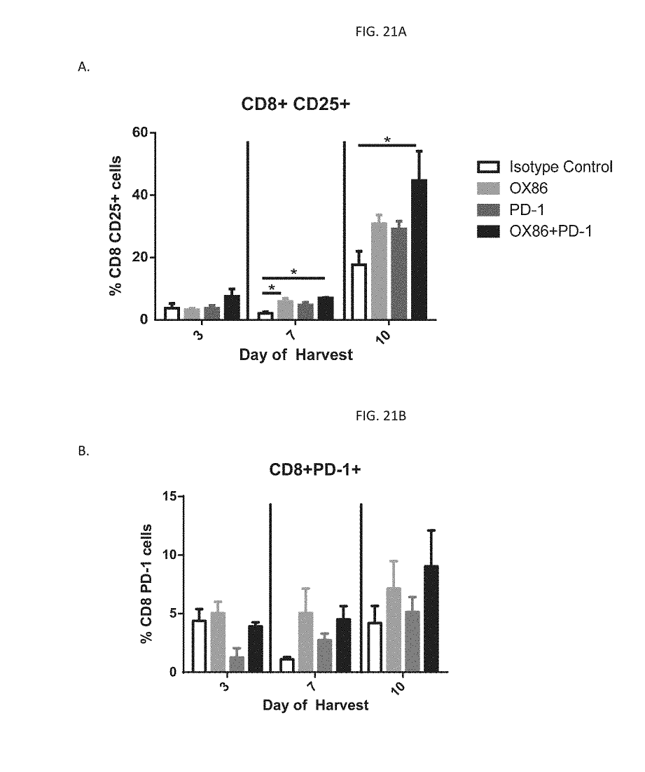

T-cells are the major cell types observed for increased populations in TILs. This effect is observed most in combination after 7 day treatment in CT26 model
Total 750 genes were analyzed by NanoString. The cutoff was based >=1.5 fold and <=0.67 of changes compare to isotypes control.
335 genes were effected by single agent or combination.
58 of them were up-regulated and 13 genes were down-regulated by OX86 alone.
8 genes were up-regulated and 36 genes were down-regulated by PD1 antibody alone.
318 genes were up-regulated and 2 genes were down-regulated by OX86 and PD1 antibody combination.
T-cells were increased in tumor environment after 7 day treatment and the best effect was observed in OX86 and PD-1 combination.
Type I interferon related genes were up-regulated in OX86 and PD1 antibody combination treatment.
Gene expression changes were also observed in:
   Immune checkpoint genes (Lag3, PD-1, PD-L1, etc.).
   MHC molecules (H2-Aa, H2-Ab1, H2-D1, H2-K1, H2-Q2, etc.)
   Transcription related genes (Maf, Runx3 and Smad2).
   Cytolysis related genes (Gzma or Gzmb).
   Chemokines (Ifn_{g}, II16, II18, II3) and integrins
   T cell signaling (Zap70) was increased.
Gene expression was analyzed by NanoString nCounter technology in which mRNAs were directly hybridized with bar-coded nCounter reporters and the signal was detected by a NanoString analyzer. A NanoString-off the shelf PanCancer_Immune profiling panel including 770 genes were used in this study. Results included: Ccl5 and Ccr5 levels increased in combination and/or OX86 treated samples (FIG. 16A and FIG. 16B)
PD-1 & PD-L1 levels increased in combination and OX86 treated samples (FIG. 17A and FIG. 17B)
IFNg, TNF-α and IL-6 levels were affected by OX86 and/or PD1 treatment (FIG. 18)
OX40/PD1 combination treatment increased proliferation (as measured by Ki67+ staining) in CD4+ and CD8+ T cells in blood in CT-26 tumor model (FIG. 19A and FIG. 19B, same legend for bars for both FIG. 19A and FIG. 19B)
OX40/PD1 combination treatment increased effector memory in CD4+ and CD8+ T cells in blood in CT-26 tumor model (FIG. 20A and FIG. 20B, same legend for bars for both FIG. 20A and FIG. 20B)
OX40/PD1 combination treatment increased activation (CD25) and PD-1 expression in CD8+ T cells in blood in CT-26 tumor model (FIG. 21A and FIG. 21B, same legend for bars for both FIG. 21A and FIG. 21B)
OX40/PD1 combination treatment increased GZB (granzyme B)+CD8+ T cells and CD8:Treg ratio in tumor in CT-26 model (FIG. 22A and FIG. 22B, same legend for bars for both FIG. 22A and FIG. 22B)

### PD TCRβ Analysis:

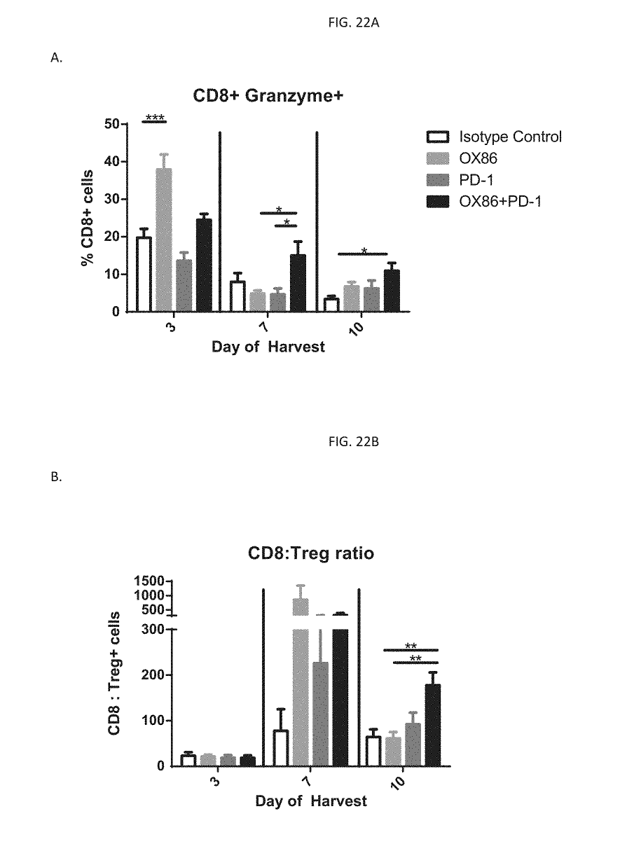

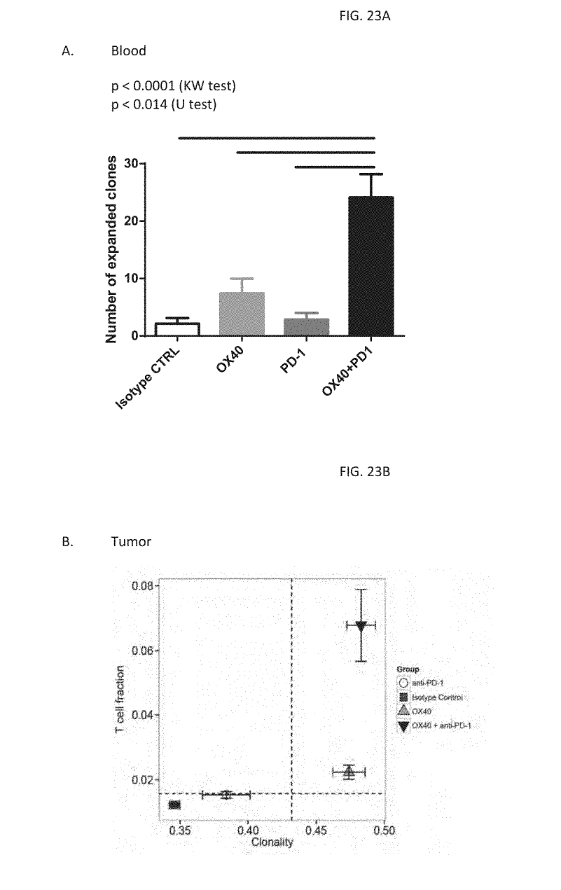

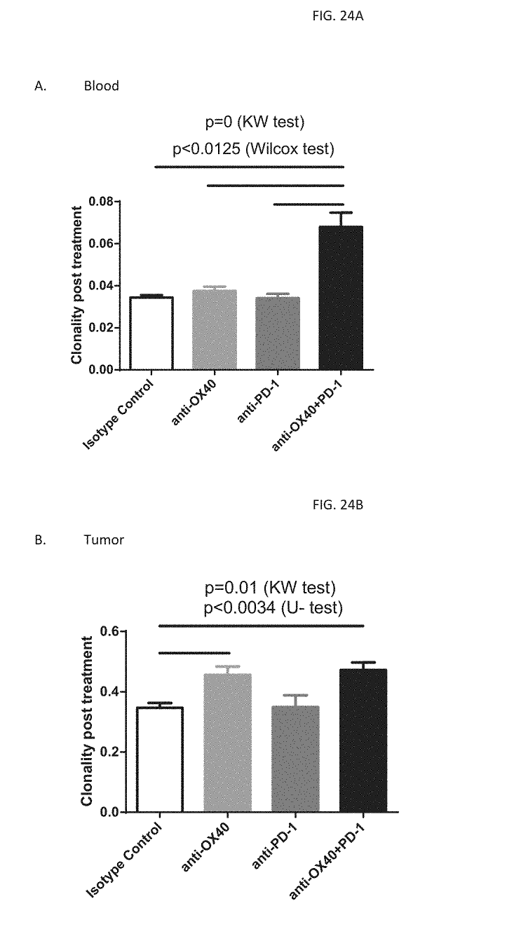

a. Anti-PD-1 mAB (monoclonal antibody) treatment enhances anti-OX40 mediated clonal expansion of T-cells and increases the clonality of T-cell repertoire in blood and tumor (FIG. 23A (blood) and FIG. 23B (tumor)).
b. There is a significant increase in clonality of TCR β repertoire in the blood following treatment with anti-OX40 and anti-PD-1 combination treatment. However, in the tumor both anti-OX40 monotherapy as well as combination significantly increases the clonality (FIG. 24A (blood) and FIG. 24B (tumor)).
c. There is a significant increase in the T-cell fraction in the tumors following anti-OX40 and anti-PD-1 treatment.

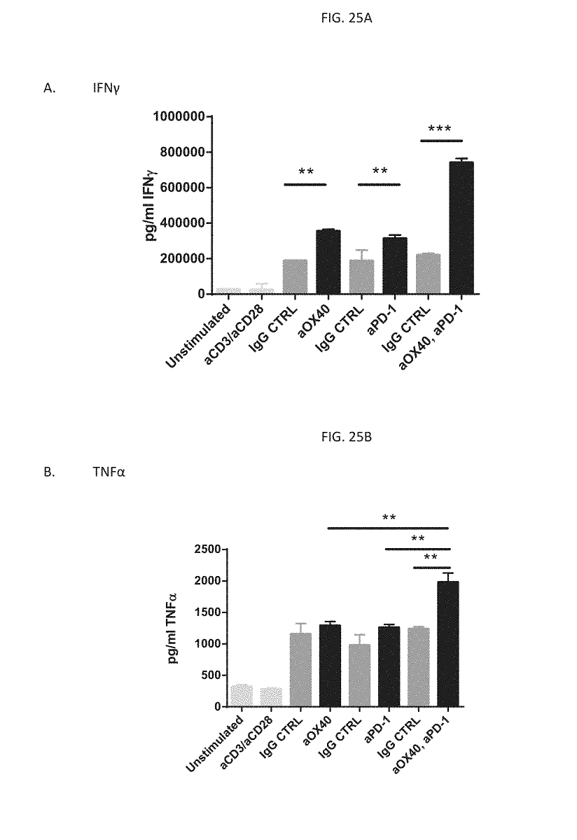

ANTIBODY 106-222 and pembrolizumab in vitro increase production of IFNγ and TNFα in human PBMC cultures (FIG. 25A and FIG. 25B)

1. A method of treating cancer in a mammal in need thereof comprising administering to the mammal a therapeutically effective amount of an antigen binding protein that binds OX40 and an antigen binding protein that binds PD-1.

2. The method of claim 1, wherein the cancer is a solid tumor.

3. The method of claim 1, wherein the cancer is selected from the group consisting of: melanoma, lung cancer, kidney cancer, breast cancer, head and neck cancer, colon cancer, ovarian cancer, pancreatic cancer, liver cancer, prostate cancer, bladder cancer, and gastric cancer.

4. The method of claim 1, wherein the cancer is a liquid tumor.

5. The method of claim 1, wherein the antigen binding protein that binds OX40 and the antigen binding protein that binds PD-1 are administered at the same time.

6. The method of claim 1, wherein the antigen binding protein that binds OX40 and the antigen binding protein that binds PD-1 are administered sequentially, in any order.

7. The method of claim 1, wherein the antigen binding protein that binds OX40 and/or the antigen binding protein that binds PD-1 are administered systemically.

8. The method of claim 1, wherein the antigen binding protein that binds OX40 and/or the antigen binding protein that binds PD-1 are administered intratumorally.

9. The method of claim 1, wherein the mammal is human.

10. The method of claim 1, wherein the tumor size of said cancer in said mammal is reduced by more than an additive amount compared with treatment with the antigen binding protein to OX40 and the antigen binding protein to PD-1 as used as monotherapy.

11. The method of claim 1, wherein the antigen binding protein that binds OX40 binds to human OX40.

12. The method of claim 1, wherein the antigen binding protein that binds to PD-1 binds to human PD-1.

13. The method of claim 1, wherein the antigen binding protein that binds OX40 and/or the antigen binding protein that binds PD-1 is a humanized monoclonal antibody.

14. The method of claim 1, wherein the antigen binding protein that binds OX40 and/or the antigen binding protein that binds PD-1 is a fully human monoclonal antibody.

15. The method of claim 1, wherein the antigen binding protein that binds OX40 and/or the antigen binding protein that binds PD-1 is an antibody with an IgG1 antibody isotype or variant thereof.

16. The method of claim 1, wherein the antigen binding protein that binds OX40 and/or the antigen binding protein that binds PD-1 is an antibody with an IgG4 antibody isotype or variant thereof.

17. The method of claim 1, wherein the antigen binding protein that binds OX40 is an agonist antibody.

18. The method of claim 1, wherein the antigen binding protein that binds PD-1 is an antagonist antibody.

19. The method of claim 1, wherein the antigen binding protein that binds OX40 comprises: a heavy chain variable region CDR1 comprising an amino acid sequence with at least 90%, 91%, 92%, 93%, 94%, 95%, 96%, 97%, 98%, 99% or 100% sequence identity to the amino acid sequence set forth in SEQ ID NO:1 or 13; a heavy chain variable region CDR2 comprising an amino acid sequence with at least 90%, 91%, 92%, 93%, 94%, 95%, 96%, 97%, 98%, 99% or 100% sequence identity to the amino acid sequence as set forth in SEQ ID NO:2 or 14; and/or a heavy chain variable region CDR3 comprising an amino acid sequence with at least 90%, 91%, 92%, 93%, 94%, 95%, 96%, 97%, 98%, 99% or 100% sequence identity to the amino acid sequence as set forth in SEQ ID NO:3 or 15.

20. The method of claim 1, wherein the antigen binding protein that binds OX40 comprises a light chain variable region CDR1 comprising an amino acid sequence with at least 90%, 91%, 92%, 93%, 94%, 95%, 96%, 97%, 98%, 99% or 100% sequence identity to the amino acid sequence as set forth in SEQ ID NO:7 or 19; a light chain variable region CDR2 comprising an amino acid sequence with at least at least 90%, 91%, 92%, 93%, 94%, 95%, 96%, 97%, 98%, 99% or 100% sequence identity to the amino acid sequence as set forth in SEQ ID NO:8 or 20 and/or a light chain variable region CDR3 comprising an amino acid sequence with at least 90%, 91%, 92%, 93%, 94%, 95%, 96%, 97%, 98%, 99% or 100% sequence identity to the amino acid sequence as set forth in SEQ ID NO:9 or 21.

21. The method of claim 1, wherein the antigen binding protein that binds OX40 comprises: (a) a heavy chain variable region CDR1 comprising the amino acid sequence of SEQ ID NO:1; (b) a heavy chain variable region CDR2 comprising the amino acid sequence of SEQ ID NO:2; (c) a heavy chain variable region CDR3 comprising the amino acid sequence of SEQ ID NO:3; (d) a light chain variable region CDR1 comprising the amino acid sequence of SEQ ID NO:7; (e) a light chain variable region CDR2 comprising the amino acid sequence of SEQ ID NO:8; and (f) a light chain variable region CDR3 comprising the amino acid sequence of SEQ ID NO:9.

22. The method of claim 1, wherein the antigen binding protein that binds OX40 comprises: (a) a heavy chain variable region CDR1 comprising the amino acid sequence of SEQ ID NO:13; (b) a heavy chain variable region CDR2 comprising the amino acid sequence of SEQ ID NO:14; (c) a heavy chain variable region CDR3 comprising the amino acid sequence of SEQ ID NO:15; (d) a light chain variable region CDR1 comprising the amino acid sequence of SEQ ID NO:19; (e) a light chain variable region CDR2 comprising the amino acid sequence of SEQ ID NO:20; and (f) a light chain variable region CDR3 comprising the amino acid sequence of SEQ ID NO:21.

23. The method of claim 1, wherein the antigen binding protein that binds OX40 comprises a light chain variable region (“VL”) comprising an amino acid sequence with at least 90%, 91%, 92%, 93%, 94%, 95%, 96%, 97%, 98%, 99% or 100% sequence identity to the amino acid sequence as set forth in SEQ ID NO:10, 11, 22 or 23.

24. The method of claim 1, wherein the antigen binding protein that binds OX40 comprises a heavy chain variable region (“VH”) comprising an amino acid sequence with at least 90%, 91%, 92%, 93%, 94%, 95%, 96%, 97%, 98%, 99% or 100% sequence identity to the amino acid sequence as set forth in SEQ ID NO:4, 5, 16 and 17.

25. The method of claim 1, wherein the antigen binding protein that binds OX40 comprises a heavy chain variable region comprising the amino acid sequence as set forth in SEQ ID NO:5 and a light chain variable region comprising the amino acid sequence as set forth in SEQ ID NO:11.

26. The method of claim 1, wherein the antigen binding protein that binds OX40 comprises a heavy chain variable region comprising the amino acid sequence as set forth in SEQ ID NO:17 and a light chain variable region comprising the amino acid sequence as set forth in SEQ ID NO:23.

27. The method of claim 1, wherein the antigen binding protein that binds OX40 comprises a light chain variable region comprising the amino acid sequence of SEQ ID NO:11 or 23, or an amino acid sequence with at least 90% sequence identity to the amino acid sequences of SEQ ID NO:11 or 23.

28. The method of claim 1, wherein the antigen binding protein that binds OX40 comprises a heavy chain variable region comprising the amino acid sequence of SEQ ID NO:5 or 17, or an amino acid sequence with at least 90% sequence identity to the amino acid sequences of SEQ ID NO:5 or 17.

29. The method of claim 1, wherein the monoclonal antibody that binds to human OX40 comprises a heavy chain comprising an amino acid sequence with at least 90%, 91%, 92%, 93%, 94%, 95%, 96%, 97%, 98%, 99% or 100% sequence identity to the amino acid sequence as set forth in SEQ ID NO:48 and a light chain comprising an amino acid sequence with at least 90%, 91%, 92%, 93%, 94%, 95%, 96%, 97%, 98%, 99% or 100% sequence identity to the amino acid sequence as set forth in SEQ ID NO:49.

30. The method of claim 1, wherein the antigen binding protein that binds PD-1 is pembrolizumab, or an antibody comprising 90%, 91%, 92%, 93%, 94%, 95%, 96%, 97%, 98%, 99% or 100% sequence identity thereto.

31. The method of claim 1, wherein the antigen binding protein that binds PD-1 is nivolumab, or an antibody having 90%, 91%, 92%, 93%, 94%, 95%, 96%, 97%, 98%, 99% or 100% sequence identity thereto.

32. The method of claim 1, wherein the mammal has increased survival when treated with a therapeutically effective amount of an antigen binding protein to OX40 and therapeutically effective amount of an antigen binding protein to PD-1 compared with a mammal who received the antigen binding protein to OX40 or the antigen binding protein to PD-1 as monotherapy.

33. The method of claim 1, further comprising administering at least one anti-neoplastic agent to the mammal in need thereof.

34. A pharmaceutical composition or kit comprising a therapeutically effective amount of an antigen binding protein that binds OX40 and a therapeutically effective amount of an antigen binding protein that binds PD-1.

35.-41. (canceled)

42. A method of reducing tumor size in a human having cancer comprising administering a therapeutically effective amount of an agonist antibody to human OX40 and a therapeutically effective amount of an antagonist antibody to human PD-1.
